(19) United States
(12) Patent Application Publication
(10) Pub. No.: US 2022/0307054 A1
(43) Pub. Date: Sep. 29, 2022

(54) MODULATION OF T CELL RESPONSES BY UL18 OF HUMAN CYTOMEGALOVIRUS

(71) Applicant: Oregon Health & Science University, Portland, OR (US)

(72) Inventors: Klaus J. FRÜH, Portland, OR (US); Scott G. HANSEN, Portland, OR (US); Daniel MALOULI, Hillsboro, OR (US); Louis J. PICKER, Portland, OR (US)

(21) Appl. No.: 17/636,506

(22) PCT Filed: Aug. 19, 2020
(86) PCT No.: PCT/US20/47050

§ 371 Date: Feb. 18, 2022

Related U.S. Application Data
(60) Provisional application No. 62/889,310, filed on Aug. 20, 2019.

(51) Int. Cl. C12N 15/86; C12N 5/0783; C07K 14/005; C07K 16/28; A61P 31/18; A61P 37/02

(52) U.S. Cl. CPC C12N 15/86; C12N 5/0638; C07K 14/005; C07K 16/2833; A61P 31/18; A61P 37/02; C12N 2710/16143; C12N 2740/15034

(57) ABSTRACT

The disclosure relates to methods of modulating T cell responses by UL18 of human cytomegalovirus. The disclosure also relates to methods of generating MHC-Ia, MHC-II, and/or MHC-E restricted CD8+ T cells.

### CROSS REFERENCE TO RELATED APPLICATIONS

This application claims the benefit of U.S. Provisional Application No. 62/889,310, filed Aug. 20, 2019, which is hereby incorporated by reference in its entirety.

### STATEMENT REGARDING FEDERALLY-SPONSORED RESEARCH AND DEVELOPMENT

This invention was made with government support under grant numbers RO1 AI059457 and U19 AI128741 awarded by the National Institute of Allergy and Infectious Disease. The government has certain rights in the invention.

### REFERENCE TO SEQUENCE LISTING SUBMITTED ELECTRONICALLY

The content of the electronically submitted sequence listing in ASCII text file (Name 4153_013PC01_Seqlisting_ST25; Size: 11,029 bytes; and Date of Creation: Aug. 19, 2020) filed with the application is incorporated herein by reference in its entirety.

### BACKGROUND OF THE INVENTION

It has been previously demonstrated that strain 68-1 of Rhesus cytomegalovirus (RhCMV) elicits CD8+ T cells that recognize peptides presented by MHC-II and MHC-E instead of conventional MHC-I. This effect was recapitulated in cynomolgus monkey CMV (CyCMV), thus demonstrating that deletion of the RhCMV and CyCMV homologs of HCMV UL128, UL130, UL146, and UL147 is required to enable the induction of MHC-E-restricted CD8+ T cells (WO 2016/130693, WO 2018/075591). In addition, these vectors elicit MHC-II restricted CD8+ T cells. However, insertion of a targeting site for the endothelial cell specific micro RNA (miR) 126 into essential viral genes of these vectors eliminates the induction of MHC-II-restricted CD8+ T cells resulting in “MHC-E only” vectors that exclusively elicit MHC-E restricted CD8+ T cells (WO 2018/075591). In contrast, insertion of the myeloid cell specific miR142-3p into 68-1 RhCMV prevents the induction of MHC-E restricted CD8+ T cells resulting in vectors that elicit CD8+ T cells exclusively restricted by MHC-II (WO 2017/087921). Similarly, deletion of the UL40 homolog Rh67 prevents the induction of MHC-E restricted CD8+ T cells resulting in “MHC-II-only vectors” (WO 2016/130693).

### BRIEF SUMMARY OF THE INVENTION

The present disclosure relates to a recombinant human CMV (HCMV) vector comprising a nucleic acid sequence encoding heterologous antigen, wherein the recombinant HCMV vector does not express UL18.

In some embodiments, the recombinant HCMV vector does not express UL128. In some embodiments, the recombinant HCMV vector does not express UL130. In some embodiments, the HCMV vector does not express UL128 and UL130.

The present disclosure also relates to a HCMV vector comprising a nucleic acid sequence encoding a heterologous antigen, wherein the recombinant HCMV vector does not express UL18, UL128, UL130, UL146, and UL147.

In some embodiments, the recombinant HCMV vector does not express UL18 protein, UL128 protein, UL protein, UL146 protein, and UL147 protein, or orthologs thereof, due to the presence of one or more mutations in the nucleic acid sequence encoding UL18, UL128, UL130, UL146, or UL147. In some embodiments, the mutations in the nucleic acid sequence encoding UL18, UL128, UL130, UL146, or UL147 are selected from the group consisting of point mutations, frameshift mutations, truncation mutations, and deletion of all of the nucleic acid sequence encoding the viral protein.

In some embodiments, the recombinant HCMV vector further comprises a nucleic acid sequence encoding UL40, or an ortholog thereof. In some embodiments, the recombinant HCMV vector further comprises a nucleic acid sequence encoding US28, or an ortholog thereof. In some embodiments, the recombinant HCMV vector does not express UL82 (pp 71), or an ortholog thereof. In some embodiments, the recombinant HCMV vector does not express US11, or an ortholog thereof.

In some embodiments, the recombinant HCMV vector further comprises a nucleic acid sequence encoding a microRNA (miRNA) recognition element (MRE), wherein the MRE contains target sites for microRNAs expressed in endothelial cells. In some embodiments, the MRE expressed in endothelial cells is miR126, miR-126-3p, miR-130a, miR-210, miR-221/222, miR-378, miR-296, and miR-328.

In some embodiments, the recombinant HCMV vector further comprises a nucleic acid sequence encoding a microRNA (miRNA) recognition element (MRE), wherein the MRE contains target sites for microRNAs expressed in myeloid cells. In some embodiments, the MRE expressed in myeloid cells is miR-142-3p, miR-223, miR-27a, miR-652, miR-155, miR-146a, miR-132, miR-21, and miR-125.

In some embodiments, the heterologous antigen is a pathogen specific antigen, a tumor antigen, a tissue specific antigen, or a host self-antigen. In some embodiments, the pathogen specific antigen is selected from the group consisting of human immunodeficiency virus (HIV), herpes simplex virus type 1, herpes simplex virus type 2, hepatitis B virus, hepatitis C virus, papillomavirus, Plasmodium parasites, and Mycobacterium tuberculosis.

In some embodiments, the pathogen specific antigen is an MHC-E supertope. In some embodiments, the MHC-E supertope is a HIV epitope. In some embodiments, the MHC-E supertope is at least 80%, at least 85%, at least 90%, at least 95%, or 100% identical to LDAWEKIRLRPGGKK (SEQ ID NO: 13); DAWEKIRLR (SEQ ID NO: 14); KKAQQAAADTGNSSQ (SEQ ID NO: 15); KAQQAAADT (SEQ ID NO: 16); QMVHQAISPRTLNAW (SEQ ID NO: 17); HQAISPRTL (SEQ ID NO: 18); NTMLNTVGGHQAAMQ (SEQ ID NO: 19); VGGHQAAMQ (SEQ ID NO: 20); STLQEQIGWMTNNPP (SEQ ID NO: 21); STLQEQIGW (SEQ ID NO: 22); IVRMYSPVSILDIRQ (SEQ ID NO: 23); RMYSPVSIL (SEQ ID NO: 24); QKQEPIDKELYPLAS (SEQ ID NO: 25); KQEPIDKEL (SEQ ID NO: 26); SFSFPQITLWQRPLV (SEQ ID NO: 27); VRQYDQILIEICGKK (SEQ ID NO: 28); EPFRKQNPDIVIYQL (SEQ ID NO: 29); YVDGAANRETKLGKA (SEQ ID NO: 30); EEHEKYSNWRAMAS (SEQ ID NO: 31); or ILDLWVYHTQGYFPD (SEQ ID NO: 32).

In some embodiments, the tumor antigen is related to a cancer selected from the group consisting of acute myelogenous leukemia, chronic myelogenous leukemia, myelodysplastic syndrome, acute lymphoblastic leukemia, chronic lymphoblastic leukemia, non-Hodgkin's lymphoma, multiple myeloma, malignant melanoma, breast cancer, lung cancer, ovarian cancer, prostate cancer, pancreatic cancer, colon cancer, renal cell carcinoma (RCC), and germ cell tumors.

In some embodiments, the host self-antigen is an antigen derived from the variable region of a T cell receptor (TCR) or an antigen derived from the variable region of a B cell receptor.

The present disclosure also relates to a pharmaceutical composition comprising the recombinant HCMV vector and a pharmaceutically acceptable carrier.

The present disclosure also relates to an immunogenic composition comprising the recombinant HCMV vector and a pharmaceutically acceptable carrier.

The present disclosure also relates to a method of generating an immune response in a subject to the at least one heterologous antigen, comprising administering to the subject the recombinant HCMV vector in an amount effective to elicit a CD8+ T cell response to the at least one heterologous antigen.

The present disclosure also relates to use of the recombinant HCMV vector in the manufacture of a medicament for use in generating an immune response in a subject.

The present disclosure also relates to the recombinant HCMV for use in generating an immune response in a subject.

The present disclosure also relates to a method of treating or preventing cancer in a subject, comprising administering the recombinant HCMV vector of in an amount effective to elicit a CD8+ T cell response to the at least one heterologous antigen.

The present disclosure also relates to the use of the recombinant HCMV vector in the manufacture of a medicament for use in treating or preventing cancer in a subject.

The present disclosure also relates to the recombinant HCMV vector for use in treating or preventing cancer in a subject.

The present disclosure also relates to a method of treating or preventing a pathogenic infection in a subject, comprising administering to the subject the recombinant HCMV vector in an amount effective to elicit a CD8+ T cell response to the at least one heterologous antigen.

The present disclosure also relates to use of the recombinant HCMV vector in the manufacture of a medicament for use in treating or preventing a pathogenic infection in a subject.

The present disclosure also relates to the recombinant HCMV vector for use in treating or preventing a pathogenic infection in a subject.

The present disclosure also relates to a method of treating an autoimmune disease or disorder in a subject, comprising administering to the subject the recombinant HCMV vector in an amount effective to elicit a CD8+ T cell response to the at least one heterologous antigen.

The present disclosure also relates to use of the recombinant HCMV vector in the manufacture of a medicament for use in treating an autoimmune disease or disorder in a subject.

The present disclosure also relates to the recombinant HCMV vector for use in treating an autoimmune disease or disorder in a subject.

In some embodiments, at least 10% of the CD8+ T cells elicited by the recombinant HCMV vector are restricted by MHC-E or an ortholog thereof. In some embodiments, at least 20%, at least 30%, at least 40%, at least 50%, at least 60%, at least 75%, at least 80%, at least 85%, at least 90%, or at least 95% of the CD8+ T cells elicited by the recombinant HCMV vector are restricted by MHC-E or an ortholog thereof.

In some embodiments, at least 10% of the CD8+ T cells elicited by the recombinant HCMV vector are restricted by MHC-II or an ortholog thereof. In some embodiments, at least 20%, at least 30%, at least 40%, at least 50%, at least 60% or at least 75% of the CD8+ T cells elicited by the recombinant HCMV vector are restricted by MHC-II or an ortholog thereof.

In some embodiments, fewer than 10%, fewer than 20%, fewer than 30%, fewer than 40%, or fewer than 50% of the CD8+ T cells elicited by the recombinant HCMV vector are restricted by MHC-class Ia or an ortholog thereof. In some embodiments, at least 10% of the CD8+ T cells elicited by the recombinant HCMV vector are restricted by MHC-class Ia or an ortholog thereof. In some embodiments, at least 20%, at least 30%, at least 40%, at least 50%, at least 60%, at least 75%, at least 80%, at least 85%, at least 90%, at least 95%, or the CD8+ T cells elicited by the recombinant HCMV vector are restricted by MHC-class Ia or an ortholog thereof.

In some embodiments, a CD8+ TCR is identified from the CD8+ T cells elicited by the recombinant HCMV vector, wherein the CD8+ TCR recognizes a MHC-II/heterologous antigen-derived peptide complex. In some embodiments, a CD8+ TCR is identified from the CD8+ T cells elicited by the HCMV vector, wherein the CD8+ TCR recognizes a MHC-E/heterologous antigen-derived peptide complex. In some embodiments, a CD8+ TCR is identified from the CD8+ T cells elicited by the HCMV vector, wherein the CD8+ TCR recognizes a MHC-class Ia/heterologous antigen-derived peptide complex.

In some embodiments, the CD8+ TCR is identified by DNA or RNA sequencing.

In some embodiments, the CD8+ TCR recognizes MHC-II supertopes.

In some embodiments, the CD8+ TCR recognizes MHC-E supertopes. In some embodiments, the MHC-E supertope is a human immunodeficiency virus epitope. In some embodiments, the MHC-E supertope is at least 80%, at least 85%, at least 90%, at least 95%, or 100% identical to the amino acid sequence of LDAWEKIRLRPGGKK (SEQ ID NO: 13); DAWEKIRLR (SEQ ID NO: 14); KKAQQAAADTGNSSQ (SEQ ID NO: 15); KAQQAAADT (SEQ ID NO: 16); QMVHQAISPRTLNAW (SEQ ID NO: 17); HQAISPRTL (SEQ ID NO: 18); NTMLNTVGGHQAAMQ (SEQ ID NO: 19); VGGHQAAMQ (SEQ ID NO: 20); STLQEQIGWMTNNPP (SEQ ID NO: 21); STLQEQIGW (SEQ ID NO: 22); IVRMYSPVSILDIRQ (SEQ ID NO: 23); RMYSPVSIL (SEQ ID NO: 24); QKQEPIDKELYPLAS (SEQ ID NO: 25); KQEPIDKEL (SEQ ID NO: 26); SFSFPQITLWQRPLV (SEQ ID NO: 27); VRQYDQILIEICGKK (SEQ ID NO: 28); EPFRKQNPDIVIYQL (SEQ ID NO: 29); YVDGAANRETKLGKA (SEQ ID NO: 30); EEHEKYSNWRAMAS (SEQ ID NO: 31); or ILDLWVYHTQGYFPD (SEQ ID NO: 32).

The present disclosure also relates to a method of generating TCR-transgenic CD8+ T cells that recognize MHC-E-peptide complexes, the method comprising: (a) administering to a first subject a recombinant HCMV vector in an amount effective to generate a set of CD8+ T cells that recognize MHC-E/peptide complexes; (b) identifying a first CD8+ TCR from the set of CD8+ T cells, wherein the first CD8+ TCR recognizes a MHC-E/heterologous antigen-derived peptide complex; (c) isolating one or more CD8+ T cells from a second subject; and (d) transfecting the one or more CD8+ T cells with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3α and CDR3β of the first CD8+ TCR, thereby generating one or more CD8+ T cells that recognize a MHC-E peptide complexes. In some embodiments, the recombinant HCMV vector does not express UL18, UL128, UL130, UL146 and/or UL147. In some embodiments, the recombinant HCMV vector does not express a UL18 protein, UL128 protein, UL130 protein, UL146 protein, and UL147 protein, or orthologs thereof, due to the presence of one or more mutations in the nucleic acid sequence encoding UL18, UL128, UL130, UL146, or UL147. In some embodiments, the mutations in the nucleic acid sequence encoding UL18, UL128, UL130, UL146, or UL147 are selected from the group consisting of point mutations, frameshift mutations, truncation mutations, and deletion of all of the nucleic acid sequence encoding the viral protein. In some embodiments, the recombinant HCMV vector further comprises a nucleic acid sequence encoding UL40, or an ortholog thereof. In some embodiments, the recombinant HCMV vector further comprises a nucleic acid sequence encoding US28, or an ortholog thereof. In some embodiments, the recombinant HCMV vector further comprises a nucleic acid sequence encoding a microRNA (miRNA) recognition element (MRE), wherein the MRE contains a target site for a miRNA expressed in endothelial cells. In some embodiments, the miRNA expressed in endothelial cells is miR126, miR-126-3p, miR-130a, miR-210, miR-221/222, miR-378, miR-296, or miR-328. In some embodiments, the heterologous antigen is a pathogen-specific antigen, a tumor antigen, a tissue-specific antigen, or a host self-antigen. In some embodiments, the pathogen-specific antigen is human immunodeficiency virus (HIV), herpes simplex virus type 1, herpes simplex virus type 2, hepatitis B virus, hepatitis C virus, papillomavirus, Plasmodium parasites, or Mycobacterium tuberculosis.

The present disclosure also relates to a method of generating TCR-transgenic CD8+ T cells that recognize MHC-E-peptide complexes, the method comprising: (a) identifying a first CD8+ TCR from a set of CD8+ T cells, wherein the set of CD8+ T cells are generated from the recombinant HCMV vector of any one of claim 5-10, 12-13, or 16-17, wherein the first CD8+ TCR recognizes a MHC-E/heterologous antigen-derived peptide complex; (b) isolating one or more CD8+ T cells from a second subject; and (c) transfecting the one or more CD8+ T cells with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3α and CDR3β of the first CD8+ TCR, thereby generating one or more TCR-transgenic CD8+ T cells that recognize MHC-E-peptide complexes. In some embodiments, the recombinant HCMV vector does not express UL18, UL128, UL130, UL146 and/or UL147. In some embodiments, the recombinant HCMV vector does not express a UL18 protein, UL128 protein, UL130 protein, UL146 protein, and UL147 protein, or orthologs thereof, due to the presence of one or more mutations in the nucleic acid sequence encoding UL18, UL128, UL130, UL146, or UL147. In some embodiments, the mutations in the nucleic acid sequence encoding UL18, UL128, UL130, UL146, or UL147 are selected from the group consisting of point mutations, frameshift mutations, truncation mutations, and deletion of all of the nucleic acid sequence encoding the viral protein. In some embodiments, the recombinant HCMV vector further comprises a nucleic acid sequence encoding UL40, or an ortholog thereof. In some embodiments, the recombinant HCMV vector further comprises a nucleic acid sequence encoding US28, or an ortholog thereof. In some embodiments, the recombinant HCMV vector further comprises a nucleic acid sequence encoding a microRNA (miRNA) recognition element (MRE), wherein the MRE contains a target site for a miRNA expressed in endothelial cells. In some embodiments, the miRNA expressed in endothelial cells is miR126, miR-126-3p, miR-130a, miR-210, miR-221/222, miR-378, miR-296, or miR-328. In some embodiments, the heterologous antigen is a pathogen-specific antigen, a tumor antigen, a tissue-specific antigen, or a host self-antigen. In some embodiments, the pathogen-specific antigen is human immunodeficiency virus (HIV), herpes simplex virus type 1, herpes simplex virus type 2, hepatitis B virus, hepatitis C virus, papillomavirus, Plasmodium parasites, or Mycobacterium tuberculosis.

In some embodiments, the first CD8+ T cell recognizes MHC-E supertopes. In some embodiments the MHC-E supertopes comprise human immunodeficiency virus epitopes. In some embodiments, the MHC-E supertope is at least 80%, at least 85%, at least 90%, at least 95%, or 100% identical to the amino acid sequence of

(SEQ ID NO: 13)
LDAWEKIRLRPGGKK;
(SEQ ID NO: 14)
DAWEKIRLR;
(SEQ ID NO: 15)
KKAQQAAADTGNSSQ;
(SEQ ID NO: 16)
KAQQAAADT;
(SEQ ID NO: 17)
QMVHQAISPRTLNAW;
(SEQ ID NO: 18)
HQAISPRTL;
(SEQ ID NO: 19)
NTMLNTVGGHQAAMQ;
(SEQ ID NO: 20)
VGGHQAAMQ;
(SEQ ID NO: 21)
STLQEQIGWMTNNPP;
(SEQ ID NO: 22)
STLQEQIGW;
(SEQ ID NO: 23)
IVRMYSPVSILDIRQ;
(SEQ ID NO: 24)
RIVIYSPVSIL;
(SEQ ID NO: 25)
QKQEPIDKELYPLAS;
(SEQ ID NO: 26)
KQEPIDKEL;
(SEQ ID NO: 27)
SFSFPQITLWQRPLV;
(SEQ ID NO: 28)
VRQYDQILIEICGKK;
(SEQ ID NO: 29)
EPFRKQNPDIVIYQL;
(SEQ ID NO: 30)
YVDGAANRETKLGKA;
(SEQ ID NO: 31)
EEHEKYSNWRAMAS;
or
(SEQ ID NO: 32)
ILDLWVYHTQGYFPD.

In some embodiments, the second CD8+ T cell recognizes MHC-E supertopes. In some embodiments, the MHC-E supertopes comprise human immunodeficiency virus epitopes. In some embodiments, the MHC-E supertope is at least 80%, at least 85%, at least 90%, at least 95%, or 100% identical to the amino acid sequence of

(SEQ ID NO: 13)
LDAWEKIRLRPGGKK;
(SEQ ID NO: 14)
DAWEKIRLR;
(SEQ ID NO: 15)
KKAQQAAADTGNSSQ;
(SEQ ID NO: 16)
KAQQAAADT;
(SEQ ID NO: 17)
QMVHQAISPRTLNAW;
(SEQ ID NO: 18)
HQAISPRTL;
(SEQ ID NO: 19)
NTMLNTVGGHQAAMQ;
(SEQ ID NO: 20)
VGGHQAAMQ;
(SEQ ID NO: 21)
STLQEQIGWMTNNPP;
(SEQ ID NO: 22)
STLQEQIGW;
(SEQ ID NO: 23)
IVRMYSPVSILDIRQ;
(SEQ ID NO: 24)
RMYSPVSIL;
(SEQ ID NO: 25)
QKQEPIDKELYPLAS;
(SEQ ID NO: 26)
KQEPIDKEL;
(SEQ ID NO: 27)
SFSFPQITLWQRPLV;
(SEQ ID NO: 28)
VRQYDQILIEICGKK;
(SEQ ID NO: 29)
EPFRKQNPDIVIYQL;
(SEQ ID NO: 30)
YVDGAANRETKLGKA;
(SEQ ID NO: 31)
EEHEKYSNWRAMAS;
or
(SEQ ID NO: 32)
ILDLWVYHTQGYFPD.

In some embodiments, the first CD8+ TCR is identified by DNA or RNA sequencing.

In some embodiments, the nucleic acid sequence encoding the second CD8+ TCR is identical to the nucleic acid sequence encoding the first CD8+ TCR.

In some embodiments, the first subject is a human. In some embodiments, the second subject is a human.

The present disclosure also relates to a method of generating CD8+ T cells that recognize MHC-E peptide complexes, the method comprising: (a) administering to a non-human primate a recombinant rhesus CMV (RhCMV) or cynomolgus CMV (CyCMV) vector deficient for orthologs of UL128, UL130, UL146, and UL147 and expressing HIV antigens in an amount effective to generate a set of CD8+ T cells that recognize MHC-E in complex with HIV supertope peptides; (b) identifying a first CD8+ TCR from the set of CD8+ T cells, wherein the first recognizes a MHC-E/supertope peptide complex; (c) isolating one or more CD8+ T cells from a second subject; and (d) transfecting the one or more CD8+ T cells with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3α and CDR3β of the first CD8+ TCR, thereby generating one or more transfected CD8+ T cells that recognize a MHC-E/heterologous antigen-derived peptide complex. In some embodimentsthe HIV epitope is at least 80%, at least 85%, at least 90%, at least 95%, or 100% identical to LDAWEKIRLRPGGKK (SEQ ID NO: 13); DAWEKIRLR (SEQ ID NO: 14); KKAQQAAADTGNSSQ (SEQ ID NO: 15); KAQQAAADT (SEQ ID NO: 16); QMVHQAISPRTLNAW (SEQ ID NO: 17); HQAISPRTL (SEQ ID NO: 18); NTMLNTVGGHQAAMQ (SEQ ID NO: 19); VGGHQAAMQ (SEQ ID NO: 20); STLQEQIGWMTNNPP (SEQ ID NO: 21); STLQEQIGW (SEQ ID NO: 22); IVRMYSPVSILDIRQ (SEQ ID NO: 23); RMYSPVSIL (SEQ ID NO: 24); QKQEPIDKELYPLAS (SEQ ID NO: 25); KQEPIDKEL (SEQ ID NO: 26); SFSFPQITLWQRPLV (SEQ ID NO: 27); VRQYDQILIEICGKK (SEQ ID NO: 28); EPFRKQNPDIVIYQL (SEQ ID NO: 29); YVDGAANRETKLGKA (SEQ ID NO: 30); EEHEKYSNWRAMAS (SEQ ID NO: 31); or ILDLWVYHTQGYFPD (SEQ ID NO: 32). The present disclosure also relates to a method of generating CD8+ T cells that recognize MHC-E peptide complexes, the method comprising: (a) identifying a first CD8+ TCR that recognizes a MHC-E/supertope peptide complex from a set of CD8+ T cells that recognize MHC-E in complex with the HIV supertope peptides, wherein the set of CD8+ T cells are generated from a recombinant rhesus (RhCMV) or cynomolgus CMV (CyCCMV) vector deficient for orthologs of UL128, UL130, UL146, and UL147 and expressing HIV antigens in an amount effective to generate the set of CD8+ T cells; (b) isolating one or more CD8+ T cells from a second subject; and (c) transfecting the one or more CD8+ T cells with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3α and CDR3β of the first CD8+ TCR, thereby generating one or more CD8+ T cells that recognize MHC-E peptide complexes. In some embodiments the HIV epitope is at least 80%, at least 85%, at least 90%, at least 95%, or 100% identical to

(SEQ ID NO: 13)
LDAWEKIRLRPGGKK;
(SEQ ID NO: 14)
DAWEKIRLR;
(SEQ ID NO: 15)
KKAQQAAADTGNSSQ;
(SEQ ID NO: 16)
KAQQAAADT;
(SEQ ID NO: 17)
QMVHQAISPRTLNAW;
(SEQ ID NO: 18)
HQAISPRTL;
(SEQ ID NO: 19)
NTMLNTVGGHQAAMQ;
(SEQ ID NO: 20)
VGGHQAAMQ;
(SEQ ID NO: 21)
STLQEQIGWMTNNPP;
(SEQ ID NO: 22)
STLQEQIGW;
(SEQ ID NO: 23)
IVRMYSPVSILDIRQ;
(SEQ ID NO: 24)
RMYSPVSIL;
(SEQ ID NO: 25)
QKQEPIDKELYPLAS;
(SEQ ID NO: 26)
KQEPIDKEL;
(SEQ ID NO: 27)
SFSFPQITLWQRPLV;
(SEQ ID NO: 28)
VRQYDQILIEICGKK;
(SEQ ID NO: 29)
EPFRKQNPDIVIYQL;
(SEQ ID NO: 30)
YVDGAANRETKLGKA;
(SEQ ID NO: 31)
EEHEKYSNWRAMAS;
or
(SEQ ID NO: 32)
ILDLWVYHTQGYFPD.

In some embodiments, the first subject is a nonhuman primate and the second subject is a human, and wherein the second CD8+ TCR is a chimeric nonhuman primate-human CD8+ TCR comprising the non-human primate CDR3α and CDR3β of the first CD8+ TCR. In some embodiments, the second CD8+ TCR comprises the non-human primate CDR1α, CDR2α, CDR3α, CDR1β, CDR2β, and CDR3β of the first CD8+ TCR. In some embodiments, the second CD8+ TCR comprises CDR1α, CDR2α, CDR3α, CDR1β, CDR2β, and CDR3β of the first CD8+ TCR. In some embodiments, the second CD8+ TCR is a chimeric CD8+ TCR.

In some embodiments, administering the recombinant HCMV vector to the first subject comprises intravenous, intramuscular, intraperitoneal, or oral administration of the recombinant HCMV vector to the first subject.

In some embodiments, the transfected CD8+ T cells are administered to the second subject to treat or prevent cancer. In some embodiments, the cancer is selected from the group consisting of acute myelogenous leukemia, chronic myelogenous leukemia, myelodysplastic syndrome, acute lymphoblastic leukemia, chronic lymphoblastic leukemia, non-Hodgkin's lymphoma, multiple myeloma, malignant melanoma, breast cancer, lung cancer, ovarian cancer, prostate cancer, pancreatic cancer, colon cancer, renal cell carcinoma (RCC), and germ cell tumors.

In some embodiments, the transfected CD8+ T cells are administered to the second subject to treat or prevent a pathogenic infection. In some embodiments, the pathogenic infection is caused by a pathogen selected from the group consisting of human immunodeficiency virus, herpes simplex virus type 1, herpes simplex virus type 2, hepatitis B virus, hepatitis C virus, papillomavirus, Plasmodium parasites, and Mycobacterium tuberculosis.

In some embodiments, the transfected CD8+ T cells are administered to the second subject to induce an autoimmune response to the host self-antigen.

The present disclosure also relates to a method of generating CD8+ T cells that recognize MHC-II-peptide complexes, the method comprising: (a) administering to a first subject the recombinant HCMV vector in an amount effective to generate a set of CD8+ T cells that recognize MHC-II/peptide complexes; (b) identifying a first CD8+ TCR from the set of CD8+ T cells, wherein the first CD8+ TCR recognizes a MHC-II/heterologous antigen-derived peptide complex; (c) isolating one or more CD8+ T cells from a second subject; and (d) transfecting the one or more CD8+ T cells with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3α and CDR3β of the first CD8+ TCR, thereby generating one or more CD8+ T cells that recognize a MHC-II peptide complex. In some embodiments, the recombinant HCMV vector comprises a nucleic acid sequence encoding heterologous antigen. In some embodiments, the recombinant HCMV vector does not express UL18. In some embodiments, the recombinant HCMV vector does not express UL128. In some embodiments, the recombinant HCMV vector does not express UL130. In some embodiments, the recombinant HCMV vector does not express UL128 and UL130. In some embodiments, the recombinant HCMV vector does not express UL146 and UL147. In some embodiments, the recombinant HCMV vector does not express a UL18 protein, UL128 protein, UL130 protein, UL146 protein, and UL147 protein, or orthologs thereof, due to the presence of one or more mutations in the nucleic acid sequence encoding UL18, UL128, UL130, UL146, or UL147. In some embodiments, the mutations in the nucleic acid sequence encoding UL18, UL128, UL130, UL146, or UL147 are selected from the group consisting of point mutations, frameshift mutations, truncation mutations, and deletion of all of the nucleic acid sequence encoding the viral protein. In some embodiments, the recombinant HCMV vector further comprises a nucleic acid sequence encoding UL40, or an ortholog thereof. In some embodiments, the recombinant HCMV vector further comprises a nucleic acid sequence encoding US28, or an ortholog thereof. In some embodiments, the recombinant HCMV vector does not express UL82 (pp 71), or an ortholog thereof. In some embodiments, the recombinant HCMV vector does not express US11, or an ortholog thereof. In some embodiments, the recombinant HCMV vector further comprises a nucleic acid sequence encoding a MRE, wherein the MRE contains a target site for a miRNA expressed in myeloid cells. In some embodiments, the miRNA expressed in myeloid cells is miR-142-3p, miR-223, miR-27a, miR-652, miR-155, miR-146a, miR-132, miR-21, or miR-125.

The present disclosure also relates to a method of generating CD8+ T cells that recognize MHC-II-peptide complexes, the method comprising: (a) identifying a first CD8+ TCR that recognizes a MHC-II/heterologous antigen-derived peptide complex from a set of CD8+ T cells that recognize MHC-II/peptide complexes, wherein the set of CD8+ T cells are generated from the recombinant HCMV vector; (b) isolating one or more CD8+ T cells from a second subject; and (c) transfecting the one or more CD8+ T cells with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3α and CDR3β of the first CD8+ TCR, thereby generating one or more CD8+ T cells that recognize a MHC-II peptide complexes. In some embodiments, the recombinant HCMV vector comprises a nucleic acid sequence encoding heterologous antigen. In some embodiments, the recombinant HCMV vector does not express UL18. In some embodiments, the recombinant HCMV vector does not express UL128. In some embodiments, the recombinant HCMV vector does not express UL130. In some embodiments, the recombinant HCMV vector does not express UL128 and UL130. In some embodiments, the recombinant HCMV vector does not express UL146 and UL147. In some embodiments, the recombinant HCMV vector does not express a UL18 protein, UL128 protein, UL130 protein, UL146 protein, and UL147 protein, or orthologs thereof, due to the presence of one or more mutations in the nucleic acid sequence encoding UL18, UL128, UL130, UL146, or UL147. In some embodiments, the mutations in the nucleic acid sequence encoding UL18, UL128, UL130, UL146, or UL147 are selected from the group consisting of point mutations, frameshift mutations, truncation mutations, and deletion of all of the nucleic acid sequence encoding the viral protein. In some embodiments, the recombinant HCMV vector further comprises a nucleic acid sequence encoding UL40, or an ortholog thereof. In some embodiments, the recombinant HCMV vector further comprises a nucleic acid sequence encoding US28, or an ortholog thereof. In some embodiments, the recombinant HCMV vector does not express UL82 (pp 71), or an ortholog thereof. In some embodiments, the recombinant HCMV vector does not express US11, or an ortholog thereof. In some embodiments, the recombinant HCMV vector further comprises a nucleic acid sequence encoding a MRE, wherein the MRE contains a target site for a miRNA expressed in myeloid cells. In some embodiments, the miRNA expressed in myeloid cells is miR-142-3p, miR-223, miR-27a, miR-652, miR-155, miR-146a, miR-132, miR-21, or miR-125.

In some embodiments, the first CD8+ T cell recognizes MHC-II supertopes. In some embodiments, the second CD8+ T cell recognizes MHC-II supertopes.

In some embodiments, the first CD8+ TCR is identified by DNA or RNA sequencing.

In some embodiments, the nucleic acid sequence encoding the second CD8+ TCR is identical to the nucleic acid sequence encoding the first CD8+ TCR.

In some embodiments, the first subject is a human. In some embodiments, the second subject is a human.

In some embodiments, administering the HCMV vector to the first subject comprises intravenous, intramuscular, intraperitoneal, or oral administration of the HCMV vector to the first subject.

In some embodiments, the transfected CD8+ T cells are administered to the second subject to treat or prevent cancer. In some embodiments, the cancer is selected from the group consisting of acute myelogenous leukemia, chronic myelogenous leukemia, myelodysplastic syndrome, acute lymphoblastic leukemia, chronic lymphoblastic leukemia, non-Hodgkin's lymphoma, multiple myeloma, malignant melanoma, breast cancer, lung cancer, ovarian cancer, prostate cancer, pancreatic cancer, colon cancer, renal cell carcinoma (RCC), and germ cell tumors.

In some embodiments, the transfected CD8+ T cells are administered to the second subject to treat or prevent a pathogenic infection. In some embodiments, the pathogenic infection is caused by a pathogen selected from the group consisting of human immunodeficiency virus, herpes simplex virus type 1, herpes simplex virus type 2, hepatitis B virus, hepatitis C virus, papillomavirus, Plasmodium parasites, and Mycobacterium tuberculosis.

In some embodiments, the transfected CD8+ T cells are administered to the second subject to induce an autoimmune response to the host self-antigen.

The present disclosure also relates to a method of generating CD8+ T cells that recognize MHC-I-peptide complexes, the method comprising: (a) administering to a first subject the recombinant HCMV vector in an amount effective to generate a set of CD8+ T cells that recognize MHC-I/peptide complexes; (b) identifying a first CD8+ TCR from the set of CD8+ T cells, wherein the first CD8+ TCR recognizes a MHC-I/heterologous antigen-derived peptide complex; (c) isolating one or more CD8+ T cells from a second subject; and (d) transfecting the one or more CD8+ T cells with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3α and CDR3β of the first CD8+ TCR, thereby generating one or more CD8+ T cells that recognize a MHC-I peptide complex. In some embodiments, the recombinant HCMV vector comprises a nucleic acid sequence encoding heterologous antigen. In some embodiments, the recombinant HCMV vector does not express UL18. In some embodiments, the recombinant HCMV vector does not express UL128. In some embodiments, the recombinant HCMV vector does not express UL130. In some embodiments, the recombinant HCMV vector does not express UL128 and UL130. In some embodiments, the recombinant HCMV vector does not express UL146 and UL147. In some embodiments, the recombinant HCMV vector does not express a UL18 protein, UL128 protein, UL130 protein, UL146 protein, and UL147 protein, or orthologs thereof, due to the presence of one or more mutations in the nucleic acid sequence encoding UL18, UL128, UL130, UL146, or UL147. In some embodiments, the mutations in the nucleic acid sequence encoding UL18, UL128, UL130, UL146, or UL147 are selected from the group consisting of point mutations, frameshift mutations, truncation mutations, and deletion of all of the nucleic acid sequence encoding the viral protein. In some embodiments, the recombinant HCMV vector further comprises a nucleic acid sequence encoding UL40, or an ortholog thereof. In some embodiments, the recombinant HCMV vector further comprises a nucleic acid sequence encoding US28, or an ortholog thereof. In some embodiments, the recombinant HCMV vector does not express UL82 (pp 71), or an ortholog thereof. In some embodiments, the recombinant HCMV vector does not express US11, or an ortholog thereof.

The present disclosure also relates to a method of generating CD8+ T cells that recognize MHC-I-peptide complexes, the method comprising: (a) identifying a first CD8+ TCR that recognizes a MHC-I/heterologous antigen-derived peptide complex from a set of CD8+ T cells that recognize a MHC-I/heterologous antigen-derived peptide complex, wherein the set of CD8+ T cells are generated from the recombinant HCMV vector of any one of claims 1-11; (b) isolating one or more CD8+ T cells from a second subject; and (c) transfecting the one or more CD8+ T cells with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3α and CDR3β of the first CD8+ TCR, thereby generating one or more CD8+ T cells that recognize MHC-I peptide complexes.

In some embodiments, the first CD8+ TCR is identified by DNA or RNA sequencing.

In some embodiments, the nucleic acid sequence encoding the second CD8+ TCR is identical to the nucleic acid sequence encoding the first CD8+ TCR.

In some embodiments, the first subject is a human. In some embodiments, the second subject is a human.

In some embodiments, the transfected CD8+ T cells are administered to the second subject to treat or prevent cancer. In some embodiments, the cancer is selected from the group consisting of acute myelogenous leukemia, chronic myelogenous leukemia, myelodysplastic syndrome, acute lymphoblastic leukemia, chronic lymphoblastic leukemia, non-Hodgkin's lymphoma, multiple myeloma, malignant melanoma, breast cancer, lung cancer, ovarian cancer, prostate cancer, pancreatic cancer, colon cancer, renal cell carcinoma (RCC), and germ cell tumors.

In some embodiments, the transfected CD8+ T cells are administered to the second subject to treat or prevent a pathogenic infection. In some embodiments, the pathogenic infection is caused by a pathogen selected from the group consisting of human immunodeficiency virus, herpes simplex virus type 1, herpes simplex virus type 2, hepatitis B virus, hepatitis C virus, papillomavirus, Plasmodium parasites, and Mycobacterium tuberculosis.

In some embodiments, the transfected CD8+ T cells are administered to the second subject to induce an autoimmune response to the host self-antigen.

In some embodiments, the pathogen specific antigen is selected from the group consisting of human immunodeficiency virus, simian immunodeficiency virus, herpes simplex virus type 1, herpes simplex virus type 2, hepatitis B virus, hepatitis C virus, papillomavirus, Plasmodium parasites, and Mycobacterium tuberculosis.

In some embodiments, the tumor antigen is related to a cancer selected from the group consisting of acute myelogenous leukemia, chronic myelogenous leukemia, myelodysplastic syndrome, acute lymphoblastic leukemia, chronic lymphoblastic leukemia, non-Hodgkin's lymphoma, multiple myeloma, malignant melanoma, breast cancer, lung cancer, ovarian cancer, prostate cancer, pancreatic cancer, colon cancer, renal cell carcinoma (RCC), and germ cell tumors.

In some embodiments, the host self-antigen is an antigen derived from the variable region of a T cell receptor (TCR) or an antigen derived from the variable region of a B cell receptor.

The present disclosure also relates to a method of treating or preventing a pathogenic infection in a subject, the method comprising administering a CD8+ T cell to the subject.

The present disclosure also relates to the use of the CD8+ T in the manufacture of a medicament for use in treating or preventing a pathogenic infection in a subject.

The present disclosure also relates to the CD8+ T cell for use in treating or preventing a pathogenic infection in a subject.

The present disclosure also relates to a method of treating or preventing cancer in a subject, the method comprising administering a CD8+ T cell to the subject.

The present disclosure also relates to use of the CD8+ T cell in the manufacture of a medicament for use in treating or preventing cancer in a subject.

The present disclosure also relates to the CD8+ T cell for use in treating or preventing cancer in a subject.

The present disclosure also relates to a method of treating an autoimmune disease or disorder, the method comprising administering a CD8+ T cell to the subject.

The present disclosure also relates to use of the CD8+ T cell in the manufacture of a medicament for use in treating an autoimmune disease or disorder.

The present disclosure also relates to the CD8+ T cell for use in treating an autoimmune disease or disorder.

The present disclosure also relates to a method of inducing an autoimmune response to a host self-antigen, the method comprising administering a CD8+ T cell to the subject.

The present disclosure also relates to a human immunodeficiency virus MHC-E supertope between 9 and 15 amino acids in length that is at least 90%, at least 95%, or 100% identical to the amino acid sequence of LDAWEKIRLRPGGKK (SEQ ID NO: 13); DAWEKIRLR (SEQ ID NO: 14); KKAQQAAADTGNSSQ (SEQ ID NO: 15); KAQQAAADT (SEQ ID NO: 16); QMVHQAISPRTLNAW (SEQ ID NO: 17); HQAISPRTL (SEQ ID NO: 18); NTMLNTVGGHQAAMQ (SEQ ID NO: 19); VGGHQAAMQ (SEQ ID NO: 20); STLQEQIGWMTNNPP (SEQ ID NO: 21); STLQEQIGW (SEQ ID NO: 22); IVRMYSPVSILDIRQ (SEQ ID NO: 23); RMYSPVSIL (SEQ ID NO: 24); QKQEPIDKELYPLAS (SEQ ID NO: 25); KQEPIDKEL (SEQ ID NO: 26); SFSFPQITLWQRPLV (SEQ ID NO: 27); VRQYDQILIEICGKK (SEQ ID NO: 28); EPFRKQNPDIVIYQL (SEQ ID NO: 29); YVDGAANRETKLGKA (SEQ ID NO: 30); EEHEKYSNWRAMAS (SEQ ID NO: 31); or ILDLWVYHTQGYFPD (SEQ ID NO: 32).

In some embodiments, the recombinant HCMV vector comprises a nucleic acid encoding one or more human immunodeficiency virus antigens. In some embodiments, the recombinant HCMV vector does not express UL128. In some embodiments, the recombinant HCMV vector does not express UL130. In some embodiments, the recombinant HCMV vector does not express UL128 and UL130. In some embodiments, the recombinant HCMV vector does not express UL146 and UL147. In some embodiments, the recombinant HCMV vector does not express UL18 protein, UL128 protein, UL130 protein, UL146 protein, and UL147 protein, or orthologs thereof, due to the presence of one or more mutations in the nucleic acid sequence encoding UL18, UL128, UL130, UL146, or UL147. In some embodiments, the mutations in the nucleic acid sequence encoding UL18, UL128, UL130, UL146, or UL147 are selected from the group consisting of point mutations, frameshift mutations, truncation mutations, and deletion of all of the nucleic acid sequence encoding the viral protein. In some embodiments, the recombinant HCMV vector further comprises a nucleic acid sequence encoding UL40, or an ortholog thereof. In some embodiments, the recombinant HCMV vector further comprises a nucleic acid sequence encoding US28, or an ortholog thereof. In some embodiments, the recombinant HCMV vector does not express UL82 (pp 71), or an ortholog thereof. In some embodiments, the recombinant HCMV vector does not express US11, or an ortholog thereof. In some embodiments, the recombinant HCMV vector further comprises a nucleic acid sequence encoding a microRNA (miRNA) recognition element (MRE), wherein the MRE contains a target site for a miRNA expressed in endothelial cells. In some embodiments, the miRNA expressed in endothelial cells is miR126, miR-126-3p, miR-130a, miR-210, miR-221/222, miR-378, miR-296, or miR-328. In some embodiments, the recombinant HCMV vector further comprises a nucleic acid sequence encoding a MRE, wherein the MRE contains a target site for a miRNA expressed in myeloid cells. In some embodiments, the miRNA expressed in myeloid cells is miR-142-3p, miR-223, miR-27a, miR-652, miR-155, miR-146a, miR-132, miR-21, or miR-125.

### BRIEF DESCRIPTION OF THE DRAWINGS/FIGURES

FIG. 1 shows the average frequencies of CD4+ or CD8+ T cells responding to SIV-antigen-derived peptide pools in the indicated cohorts. T cell frequencies were determined in peripheral blood mononuclear cells (PBMC) at the indicated time points by intracellular cytokine staining (ICS) for IFNγ or TNFα in the presence of pools of overlapping (by 11A) 15mer peptides representing the SIV antigens. Cohort 1 was immunized with three “MHC-E only” 68-1 RhCMV vectors carrying recognition sites for mir126 in the 3′ untranslated region of the essential genes Rh108 (UL79) and Rh156 (IE2) and expressing the SIV antigens SIVgag, SIVretanef (fusion of rev, tat, and nef), or the 5′ segment of SIVpol. Cohort 2 was immunized with three “MHC-II only” 68-1 RhCMV vectors deleted for Rh67 (UL40) and expressing the SIV antigens SIVgag, SIVretanef, or the 5′ segment of SIVpol. Cohort 3 was immunized with three “MHC-II only” 68-1 RhCMV vectors carrying recognition sites for mir142 in the 3′ untranslated region of the essential genes Rh108 (UL79) and Rh156 (IE2) and expressing the SIV antigens SIVgag, SIVretanef, or the 5′ segment of SIVpol. Cohort 4 (control cohort) was immunized with three 68-1 RhCMV vectors expressing the SIV antigens SIVgag, SIVretanef, or the 5′ segment of SIVpol.

FIG. 2 shows SIVgag-specific CD8+ T cell responses in PBMC obtained from three Rhesus macaques (RM) in each of the indicated cohorts measured in the presence of individual peptides. Peptides resulting in specific CD8+ T cell responses are indicated by a box, with the color of the box designating MHC restriction as determined by blocking with the anti-pan-MHC-I mAb W6/32, the MHC-E blocking peptide VL9 and the MHC-II blocking peptide CLIP.

FIG. 3 shows plasma viral load after repeated limiting dose SIVmac239 challenge of RM in cohorts 1, 2, and 3 (left panel) and SIVvif specific CD8+ T cell responses of RM in cohorts 1, 2, and 3 (right panel). Animals that controlled SIV infection (RM controllers) are shown in white boxes and non-controllers are shown in black boxes. One animal in cohort 2 initially controlled SIV infection, but control was lost upon depletion of CD8+ T cells consistent with this RM being a spontaneous elite controller.

FIG. 4 shows an immunoblot of the SIV supertope fusion construct. Telomerized rhesus fibroblasts (TRF) were infected, or non-infected, with the indicated RhCMV constructs and lysates of infected cells were electrophoretically separated prior to immunoblotting. The SIV supertope-containing fusion protein was visualized with an anti-HA antibody whereas viral protein IE1, Rh107, and Rh108 were detected using specific antibodies. The protein band observed in mock-infected or uninfected TRF lysates with IE antibodies is non-specific.

FIG. 5A shows the average frequencies of CD8+ T cells responding to SIV-antigen derived peptides in PBMC of cohort 5 animals (n=8). Cohort 5 was immunized with a 68-1 RhCMV vector carrying recognition sites for mir126 in the 3′ untranslated region of the essential genes Rh108 (UL79) and Rh156 (IE2) and expressing the MHC-E supertope fusion protein. T cell frequencies were determined in peripheral blood mononuclear cells (PBMC) at the indicated time points by intracellular cytokine staining (ICS) for IFNγ or TNFα in the presence of pools of individual 15mer peptides representing the SIV supertopes. FIG. 5B shows the frequencies of CD8+ T cells responding to specific MHC-E restricted supertopes in individual RM. MHC-E restricted supertopes (Gag69 and Gag120) and other MHC-E restricted Gag epitopes are shown.

FIG. 6 shows SIV plasma viral load after repeated limiting dose SIVmac239 challenge of RM in cohort 5 (left panel) and SIVvif specific T cell responses (right panel). RM controllers are shown in white boxes and non-controllers are shown in black boxes. SIVvif-specific responses demonstrate “take” of SIV infection in controller animals.

FIG. 7A shows the frequencies of CD8+ T cells responding to SIV antigen peptide pools in RM inoculated with 68-1 RhCMV expressing SIVgag (n=2), 68-1 RhCMV expressing UL18 and SIVretanef (n=2), or 68-1 RhCMV expressing UL18 and SIVpol (n=2). FIG. 7B shows the frequencies of CD8+ T cells responding to MHC-E restricted supertopes in each RM. FIG. 7C shows the frequencies of CD8+ T cells responding to MHC-II restricted supertopes in each RM.

FIG. 8 shows SIVpol specific CD8+ T cells responses in PBMC obtained from three RM inoculated with 68-1 RhCMV expressing UL18 and SIVpol. CD8+ T cell responses were measured in the presence of individual peptides. Peptides resulting in specific CD8+ T cell responses are indicated by a box, with the color of the box designating MHC restriction as determined by blocking with the anti-pan-MHC-I-mAb W6/32, the MHC-E blocking peptide VL9 and the MHC-II blocking peptide CLIP. All peptide responses were blocked with W6/32 but not by VL9 peptide or CLIP peptide. Thus, CD8+ T cells are exclusively restricted by MHC-I.

FIG. 9A shows a dot plot depicting the frequencies of CD8+ T cells producing IFNγ or TNFα in response to SIVpol peptides from an RM inoculated with 68-1 RhCMV expressing UL18 and SIVpol. FIG. 9B shows a dot plot depicting the frequencies of CD8+ T cells producing IFNγ or TNFα in response to SIVpol peptides from an RM inoculated with 68-1 RhCMV expressing the UL18 D196S mutant and SIVpol. The frequencies of CD8+ T cells responding to pools of overlapping 15mer peptides comprising SIVpol or the MHC-E restricted supertope peptide SIVpol41 or the MHC-II-restricted supertope peptide SIVpol90 is shown. Whereas intact UL18 prevents the induction of supertope responses, this is not observed for the D196S mutant of UL18.

FIG. 10 shows an immunoblot of human MRCS fibroblasts uninfected or infected with HCMV-TR3 (Caposio P. et al. 2019. Characterization of a live-attenuated HCMV-based vaccine platform. Sci Rep 9:19236) or with a HCMV-TR3-based vectors in which UL18 was replaced with a HIVgag, HIVnef and HIVpol fusion protein. In addition, the UL18-deleted vector lacked UL128, UL130, UL146 and UL147 since previous work has shown that these genes inhibit MHC-E restricted CD8+ T cell responses (U.S. Pat. No. 10,532,099). In addition, the p24 fragment of HIVgag was added for control. The upper blot was probed with antibodies to the HIVgag protein. The lower blot was probed with antibodies to the HCMV pp65 protein.

FIG. 11 shows HIV gag, nef and pol-specific CD8+ T cells responses in PBMC obtained from RM inoculated the UL18-deleted vector (FIG. 11, n=2). CD8+ T cell responses were measured on day 56 post-vaccination using overlapping peptide pools corresponding to each portion of the antigen.

### DETAILED DESCRIPTION OF THE INVENTION

### I. Terms

Unless otherwise noted, technical terms are used according to conventional usage.

All publications, patents, patent applications, internet sites, and accession numbers/database sequences (including both polynucleotide and polypeptide sequences) cited herein or listed in the Application Data Sheet, including U.S. Provisional Patent Application No. 62/889,310 filed Aug. 20, 2019, are hereby incorporated by reference in their entirety for all purposes to the same extent as if each individual publication, patent, patent application, internet site, or accession number/database sequence were specifically and individually indicated to be so incorporated by reference.

Although methods and materials similar or equivalent to those described herein may be used in the practice or testing of this disclosure, suitable methods and materials are described below. In addition, the materials, methods, and examples are illustrative only and not intended to be limiting. In order to facilitate review of the various embodiments of the disclosure, the following explanations of specific terms are provided.

Unless the context requires otherwise, throughout the present specification and claims, the word “comprise” and variations thereof, such as “comprises” and “comprising,” are to be construed in an open, inclusive sense, that is, as “including, but not limited to”. “Consisting of” shall mean excluding more than trace elements of other ingredients and substantial method steps disclosed herein. The term “consisting essentially of” limits the scope of a claim to the specified materials or steps, or to those that do not materially affect the basic characteristics of a claimed invention. For example, a composition consisting essentially of the elements as defined herein would not exclude trace contaminants from the isolation and purification method and pharmaceutically acceptable carriers, such as phosphate buffered saline, preservatives, and the like. Similarly, a protein consists essentially of a particular amino acid sequence when the protein includes additional amino acids that contribute to at most 20% of the length of the protein and do not substantially affect the activity of the protein (e.g., alters the activity of the protein by no more than 50%). Embodiments defined by each of the transitional terms are within the scope of this invention.

Antigen: As used herein, the terms “antigen” or “immunogen” are used interchangeably to refer to a substance, typically a protein, which is capable of inducing an immune response in a subject. The term also refers to proteins that are immunologically active in the sense that once administered to a subject (either directly or by administering to the subject a nucleotide sequence or vector that encodes the protein) the protein is able to evoke an immune response of the humoral and/or cellular type directed against that protein.

Antigen-specific T cell: A CD8⁺ or CD4⁺ lymphocyte that recognizes a particular antigen. Generally, antigen-specific T cells specifically bind to a particular antigen presented by MHC molecules, but not other antigens presented by the same MHC.

Administration: As used herein, the term “administration” means to provide or give a subject an agent, such as a composition comprising an effective amount of a CMV vector comprising an exogenous antigen by any effective route. Exemplary routes of administration include, but are not limited to, injection (such as subcutaneous, intramuscular, intradermal, intraperitoneal, and intravenous), oral, sublingual, rectal, transdermal, intranasal, vaginal and inhalation routes.

Effective amount: As used herein, the term “effective amount” refers to an amount of an agent, such as a CMV vector comprising a heterologous antigen or a transfected CD8+ T cell that recognizes a MHC-E/heterologous antigen-derived peptide complex, a MHC-II/heterologous antigen-derived peptide complex, or a MHC-I/heterologous antigen-derived peptide complex, that is sufficient to generate a desired response, such as reduce or eliminate a sign or symptom of a condition or disease or induce an immune response to an antigen. In some examples, an “effective amount” is one that treats (including prophylaxis) one or more symptoms and/or underlying causes of any of a disorder or disease. An effective amount may be a therapeutically effective amount, including an amount that prevents one or more signs or symptoms of a particular disease or condition from developing, such as one or more signs or symptoms associated with infectious disease or cancer.

Heterologous antigen: As used herein, the term “heterologous antigen” refers to any protein or fragment thereof that is not derived from CMV. Heterologous antigens may be pathogen-specific antigens, tumor virus antigens, tumor antigens, host self-antigens, or any other antigen.

Hyperproliferative disease: A disease or disorder characterized by the uncontrolled proliferation of cells. Hyperproliferative diseases include, but are not limited to malignant and non-malignant tumors.

Immune tolerance: As used herein “immune tolerance” refers to a state of unresponsiveness of the immune system to substances that have the potential to induce an immune response. Self-tolerance to an individual's own antigens, for example, tumor antigens, is achieved through both central tolerance and peripheral tolerance mechanisms.

Immunogenic peptide: A peptide which comprises an allele-specific motif or other sequence, such as an N-terminal repeat, such that the peptide will bind an MHC molecule and induce a cytotoxic T lymphocyte (“CTL”) response, or a B cell response (for example antibody production) against the antigen from which the immunogenic peptide is derived.

In some embodiments, immunogenic peptides are identified using sequence motifs or other methods, such as neural net or polynomial determinations known in the art. Typically, algorithms are used to determine the “binding threshold” of peptides to select those with scores that give them a high probability of binding at a certain affinity and will be immunogenic. The algorithms are based either on the effects on MHC binding of a particular amino acid at a particular position, the effects on antibody binding of a particular amino acid at a particular position, or the effects on binding of a particular substitution in a motif-containing peptide. Within the context of an immunogenic peptide, a “conserved residue” is one which appears in a significantly higher frequency than would be expected by random distribution at a particular position in a peptide. In some embodiments, a conserved residue is one where the MHC structure may provide a contact point with the immunogenic peptide.

MicroRNA: As used herein, the term “microRNA” refers to a major class of biomolecules involved in control of gene expression. For example, in human heart, liver or brain, miRNAs play a role in tissue specification or cell lineage decisions in addition, miRNAs influence a variety of processes, including early development, cell proliferation and ceil death, and apoptosis and fat metabolism. The large number of miRNA genes, the diverse expression patterns, and the abundance of potential miRNA targets suggest that miRNAs may be a significant source of genetic diversity.

A mature miRNA is typically an 8-25 nucleotide non-coding RNA that regulates expression of an mRNA including sequences complementary to the miRNA. These small RNA molecules are known to control gene expression by regulating the stability and/or translation of mRNAs. For example, miRNAs bind to the 3′ UTR of target mRNAs and suppress translation. MiRNAs may also bind to target mRNAs and mediate gene silencing through the RNAi pathway. MiRNAs may also regulate gene expression by causing chromatin condensation.

A miRNA silences translation of one or more specific mRNA molecules by binding to a miRNA recognition element (MRE,) which is defined as any sequence that directly base pairs with and interacts with the miRNA somewhere on the mRNA transcript. Often, the MRE is present in the 3′ untranslated region (UTR) of the mRNA, but it may also be present in the coding sequence or in the 5′ UTR. MREs are not necessarily perfect complements to miRNAs, usually having only a few bases of complementarity to the miRNA and often containing one or more mismatches within those bases of complementarity. The MRE may be any sequence capable of being bound by a miRNA sufficiently that the translation of a gene to which the MRE is operably linked (such as a CMV gene that is essential or augmenting for growth in vivo) is repressed by a miRNA silencing mechanism such as the RISC.

Mutation: As used herein, the term “mutation” refers to any difference in a nucleic acid or polypeptide sequence from a normal, consensus, or “wild type” sequence. A mutant is any protein or nucleic acid sequence comprising a mutation. In addition, a cell or an organism with a mutation may also be referred to as a mutant. Some types of coding sequence mutations include point mutations (differences in individual nucleotides or amino acids); silent mutations (differences in nucleotides that do not result in an amino acid changes); deletions (differences in which one or more nucleotides or amino acids are missing, up to and including a deletion of the entire coding sequence of a gene); frameshift mutations (differences in which deletion of a number of nucleotides indivisible by 3 results in an alteration of the amino acid sequence). A mutation that results in a difference in an amino acid may also be called an amino acid substitution mutation. Amino acid substitution mutations may be described by the amino acid change relative to wild type at a particular position in the amino acid sequence.

Nucleotide sequences or nucleic acid sequences: The terms “nucleotide sequences” and “nucleic acid sequences” refer to deoxyribonucleic acid (DNA) or ribonucleic acid (RNA) sequences, including, without limitation, messenger RNA (mRNA), DNA/RNA hybrids, or synthetic nucleic acids. The nucleic acid may be single-stranded, or partially or completely double stranded (duplex). Duplex nucleic acids may be homoduplex or heteroduplex.

Operably Linked: As the term “operably linked” is used herein, a first nucleic acid sequence is operably linked with a second nucleic acid sequence when the first nucleic acid sequence is placed in such a way that it has an effect upon the second nucleic acid sequence. Operably linked DNA sequences may be contiguous, or they may operate at a distance.

Promoter: As used herein, the term “promoter” may refer to any of a number of nucleic acid control sequences that directs transcription of a nucleic acid. Typically, a eukaryotic promoter includes necessary nucleic acid sequences near the start site of transcription, such as, in the case of a polymerase II type promoter, a TATA element or any other specific DNA sequence that is recognized by one or more transcription factors. Expression by a promoter may be further modulated by enhancer or repressor elements. Numerous examples of promoters are available and well known to those of ordinary skill in the art. A nucleic acid comprising a promoter operably linked to a nucleic acid sequence that codes for a particular polypeptide may be termed an expression vector.

Recombinant: As used herein, the term “recombinant” with reference to a nucleic acid or polypeptide refers to one that has a sequence that is not naturally occurring or has a sequence that is made by an artificial combination of two or more otherwise separated segments of sequence, for example a CMV vector comprising a heterologous antigen. This artificial combination is often accomplished by chemical synthesis or, more commonly, by the artificial manipulation of isolated segments of nucleic acids, e.g., by genetic engineering techniques. A recombinant polypeptide may also refer to a polypeptide that has been made using recombinant nucleic acids, including recombinant nucleic acids transferred to a host organism that is not the natural source of the polypeptide (for example, nucleic acids encoding polypeptides that form a CMV vector comprising a heterologous antigen).

Pharmaceutically acceptable carriers: As used herein, a “pharmaceutically acceptable carrier” of use is conventional. Remington's Pharmaceutical Sciences, by E. W. Martin, Mack Publishing Co., Easton, Pa., 19th Edition, 1995, describes compositions and formulations suitable for pharmaceutical delivery of the compositions disclosed herein. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol, or the like as a vehicle. For solid compositions (such as powder, pill, tablet, or capsule forms), conventional non-toxic solid carriers may include, for example, pharmaceutical grades of mannitol, lactose, starch, or magnesium stearate. In addition to biologically neutral carriers, pharmaceutical compositions to be administered may contain minor amounts of non-toxic auxiliary substances, such as wetting or emulsifying agents, preservatives, and pH buffering agents and the like, for example sodium acetate or sorbitan monolaurate.

Polynucleotide: As used herein, the term “polynucleotide” refers to a polymer of ribonucleic acid (RNA) or deoxyribonucleic acid (DNA). A polynucleotide is made up of four bases; adenine, cytosine, guanine, and thymine/uracil (uracil is used in RNA). A coding sequence from a nucleic acid is indicative of the sequence of the protein encoded by the nucleic acid.

Polypeptide: The terms “protein”, “peptide”, “polypeptide”, and “amino acid sequence” are used interchangeably herein to refer to polymers of amino acid residues of any length. The polymer may be linear or branched, it may comprise modified amino acids or amino acid analogs, and it may be interrupted by chemical moieties other than amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling or bioactive component.

Orthologs of proteins are typically characterized by possession of greater than 75% sequence identity counted over the full-length alignment with the amino acid sequence of specific protein using ALIGN set to default parameters. Proteins with even greater similarity to a reference sequence will show increasing percentage identities when assessed by this method, such as at least 80%, at least 85%, at least 90%, at least 92%, at least 95%, or at least 98% sequence identity. In addition, sequence identity can be compared over the full length of particular domains of the disclosed peptides.

Sequence identity/similarity: As used herein, the identity/similarity between two or more nucleic acid sequences, or two or more amino acid sequences, is expressed in terms of the identity or similarity between the sequences. Sequence identity may be measured in terms of percentage identity; the higher the percentage, the more identical the sequences are. Sequence similarity may be measured in terms of percentage identity or similarity (which takes into account conservative amino acid substitutions); the higher the percentage, the more similar the sequences are. Polypeptides or protein domains thereof that have a significant amount of sequence identity and also function the same or similarly to one another (for example, proteins that serve the same functions in different species or mutant forms of a protein that do not change the function of the protein or the magnitude thereof) may be called “homologs.”

Methods of alignment of sequences for comparison are well known in the art. Various programs and alignment algorithms are described in: Smith & Waterman, Adv Appl Math 2, 482 (1981); Needleman & Wunsch, J Mol Biol 48, 443 (1970); Pearson & Lipman, Proc Natl Acad Sci USA 85, 2444 (1988); Higgins & Sharp, Gene 73, 237-244 (1988); Higgins & Sharp, CABIOS 5, 151-153 (1989); Corpet et al, Nuc Acids Res 16, 10881-10890 (1988); Huang et al, Computer App Biosci 8, 155-165 (1992); and Pearson et al, Meth Mot Bio 24, 307-331 (1994). In addition, Altschul et al, J Mol Biol 215, 403-410 (1990), presents a detailed consideration of sequence alignment methods and homology calculations.

The NCBI Basic Local Alignment Search Tool (BLAST) (Altschul et al, (1990) supra) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn and tblastx. Additional information may be found at the NCBI web site.

BLASTN is used to compare nucleic acid sequences, while BLASTP is used to compare amino acid sequences. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1154 nucleotides is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (that is, 15÷20*100=75).

For comparisons of amino acid sequences of greater than about 30 amino acids, the Blast 2 sequences function is employed using the default BLOSUM62 matrix set to default parameters, (gap existence cost of 11, and a per residue gap cost of 1). Homologs are typically characterized by possession of at least 70% sequence identity counted over the full-length alignment with an amino acid sequence using the NCBI Basic Blast 2.0, gapped blastp with databases such as the nr database, swissprot database, and patented sequences database. Queries searched with the blastn program are filtered with DUST (Hancock & Armstrong, Comput Appl Biosci 10, 67-70 (1994.) Other programs use SEG. In addition, a manual alignment may be performed. Proteins with even greater similarity will show increasing percentage identities when assessed by this method, such as at least about 75%, 80%, 85%, 90%, 95%, 98%, or 99% sequence identity to a protein.

When aligning short peptides (fewer than around 30 amino acids), the alignment is performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least about 60%, 70%, 75%, 80%, 85%, 90%, 95%, 98%, or 99% sequence identity to a protein. When less than the entire sequence is being compared for sequence identity, homologs will typically possess at least 75% sequence identity over short windows of 10-20 amino acids, and may possess sequence identities of at least 85%, 90%, 95% or 98% depending on their identity to the reference sequence. Methods for determining sequence identity over such short windows are described at the NCBI web site.

One indication that two nucleic acid molecules are closely related is that the two molecules hybridize to each other under stringent conditions, as described above. Nucleic acid sequences that do not show a high degree of identity may nevertheless encode identical or similar (conserved) amino acid sequences, due to the degeneracy of the genetic code. Changes in a nucleic acid sequence may be made using this degeneracy to produce multiple nucleic acid molecules that all encode substantially the same protein. Such homologous nucleic acid sequences can, for example, possess at least about 50%, 60%, 70%, 80%, 90%, 95%, 98%, or 99% sequence identity to a nucleic acid that encodes a protein.

Subject: As used herein, the term “subject” refers to a living multi-cellular vertebrate organisms, a category that includes both human and non-human mammals.

Supertope: As used herein, the term “supertope” or “supertope peptide” refers to an epitope or peptide that is recognized by T cells in greater than about 90% of the human population regardless of MHC haplotype, i.e., in the presence or absence of given MHC-I, MHC-II, or MHC-E alleles.

Treatment: As used herein, the term “treatment” refers to an intervention that ameliorates a sign or symptom of a disease or pathological condition. As used herein, the terms “treatment”, “treat”, and “treating,” with reference to a disease, pathological condition or symptom, also refers to any observable beneficial effect of the treatment. The beneficial effect may be evidenced, for example, by a delayed onset of clinical symptoms of the disease in a susceptible subject, a reduction in severity of some or all clinical symptoms of the disease, a slower progression of the disease, a reduction in the number of relapses of the disease, an improvement in the overall health or well-being of the subject, or by other parameters well known in the art that are specific to the particular disease. A prophylactic treatment is a treatment administered to a subject who does not exhibit signs of a disease or exhibits only early signs, for the purpose of decreasing the risk of developing pathology. A therapeutic treatment is a treatment administered to a subject after signs and symptoms of the disease have developed.

Vaccine: An immunogenic composition that can be administered to a mammal, such as a human, to confer immunity, such as active immunity, to a disease or other pathological condition. Vaccines can be used prophylactically or therapeutically. Thus, vaccines can be used reduce the likelihood of developing a disease (such as a tumor or pathological infection) or to reduce the severity of symptoms of a disease or condition, limit the progression of the disease or condition (such as a tumor or a pathological infection), or limit the recurrence of a disease or condition (such as a tumor). In particular embodiments, a vaccine is a replication-deficient CMV expressing a heterologous antigen, such as a tumor associated antigen derived from a tumor of the lung, prostate, ovary, breast, colon, cervix, liver, kidney, bone, or a melanoma.

Vector: Nucleic acid molecules of particular sequence can be incorporated into a vector that is then introduced into a host cell, thereby producing a transformed host cell. A vector may include nucleic acid sequences that permit it to replicate in a host cell, such as an origin of replication. A vector may also include one or more selectable marker genes and other genetic elements known in the art, including promoter elements that direct nucleic acid expression. Vectors can be viral vectors, such as CMV vectors. Viral vectors may be constructed from wild type or attenuated virus, including replication deficient virus.

### II. Methods for the Modulation of T Cell Responses by UL18 of HCMV

Disclosed herein are methods for the modulation of T cell responses by UL18 of HCMV. The methods involve administering an effective amount of at least one recombinant HCMV vector comprising at least one heterologous antigen to a subject, wherein the HCMV vector does not express UL18.

In some embodiments, the method further comprises generating an immune response to the at least one heterologous antigen, comprising administering to the subject the HCMV vector in an amount effective to elicit a CD8+ T cell response to the at least one heterologous antigen. In some embodiments, the method further comprises treating or preventing cancer in a subject, comprising administering the HCMV vector in an amount effective to elicit a CD8+ T cell response to the at least one heterologous antigen. In some embodiments, the method further comprises treating or preventing a pathogenic infection in a subject, comprising administering the HCMV vector in an amount effective to elicit a CD8+ T cell response to the at least one heterologous antigen. In some embodiments, the method further comprises treating an autoimmune disease or disorder in a subject, comprising administering to the subject the HCMV vector in an amount effective to elicit a CD8+ T cell response to the at least one heterologous antigen.

In some embodiments, the UL18-deficient HCMV vector also does not express an UL128, UL130, UL146, or UL147 protein due to the presence of a mutation in the nucleic acid sequence encoding UL128, UL130, UL146, or UL147. In addition, any of the UL18-deficient HCMV vectors can be deficient for US11, and/or UL82 protein due to the presence of a mutation in the nucleic acid sequence encoding US11, and/or UL82. The mutation may be any mutation that results in a lack of expression of active proteins. Such mutations may include point mutations, frameshift mutations, deletions of less than all of the sequence that encodes the protein (truncation mutations), or deletions of all of the nucleic acid sequence that encodes the protein, or any other mutations.

In some embodiments, the HCMV vector lacks UL18, UL128, UL130, UL146, and UL147 and expresses UL40 and US28.

In some embodiments, the HCMV vector comprises a nucleic acid sequence encoding a microRNA (miRNA) recognition element (MRE). In some embodiments, the HCMV vector lacks UL18, UL128, UL130, UL146, and UL147 (and optionally UL82) and expresses UL40 and US28 and the MRE contains target sites for microRNAs expressed in endothelial cells. Examples of such miRNAs expressed in endothelial cells are miR126, miR-126-3p, miR-130a, miR-210, miR-221/222, miR-378, miR-296, and miR-328. In some embodiments, the HCMV vector lacks UL18 and the MRE contains target sites for microRNAs expressed in myeloid cells. Examples of such miRNAs expressed in myeloid cells are miR-142-ep, miR-223, miR-27a, miR-652, miR-155, miR-146a, miR-132, miR-21, and miR-125.

The MRE may be any miRNA recognition element that silences expression in the presence of a miRNA expressed by endothelial cells. The MRE may be any miRNA recognition element that silences expression in the presence of a miRNA expressed by myeloid cells. Such an MRE may be the exact complement of a miRNA. Alternatively, other sequences may be used as MREs for a given miRNA. For example, MREs may be predicted from sequences. In one example, the miRNA may be searched on the website microRNA.org (www.microrna.org). In turn, a list of mRNA targets of the miRNA will be listed. For each listed target on the page, ‘alignment details’ may be accessed and putative MREs accessed.

One of ordinary skill in the art may select a validated, putative, or mutated MRE sequence from the literature that would be predicted to induce silencing in the presence of a miRNA expressed in a myeloid cell such as a macrophage. One example involves the above referenced website. The person of ordinary skill in the art may then obtain an expression construct whereby a reporter gene (such as a fluorescent protein, enzyme, or other reporter gene) has expression driven by a promoter such as a constitutively active promoter or cell specific promoter. The MRE sequence may then be introduced into the expression construct. The expression construct may be transfected into an appropriate cell, and the cell transfected with the miRNA of interest. A lack of expression of the reporter gene indicates that the MRE silences gene expression in the presence of the miRNA.

In some embodiments, the heterologous antigen may be a pathogen specific antigen, a tumor antigen, a tumor specific antigen, or a host self-antigen. In some embodiments, the host self-antigen is derived from the variable region of a T cell receptor (TCR) or an antigen derived from the variable region of a B cell receptor.

The pathogen specific antigen may be derived from, for example, human immunodeficiency virus, simian immunodeficiency virus, herpes simplex virus type 1, herpes simplex virus type 2, hepatitis B virus, hepatitis C virus, papillomavirus, Plasmodium parasites, Clostridium tetani, and Mycobacterium tuberculosis.

Tumor antigens are relatively restricted to tumor cells and can be any protein that induces an immune response. However, many tumor antigens are host (self) proteins and thus are typically not seen as antigenic by the host immune system. Tumor antigens can also be abnormally expressed by cancer cells. Tumor antigens can also be germline/testis antigens expressed in cancer cells, cell lineage differentiation antigens not expressed in adult tissue, or antigens overexpressed in cancer cells. Tumor antigens include, but are not limited to, prostatic acidic phosphatase (PAP); Wilms tumor suppressor protein (WT1); Mesothelin (MSLN); Her-2 (HER2); human papilloma virus antigen E6 of strain HPV16; human papilloma virus antigen E7 of strain HPV16; human papilloma virus antigen E6 of strain HPV18; Human papilloma virus antigen E7 of strain HPV18; a fusion protein of human papilloma virus E6 and E7 from HPV16 and HPV18; mucin 1 (MUC1); LMP2; epidermal growth factor receptor (EGFR); p53; New York esophagus 1 (NY-ESO-1); prostate specific membrane antigen (PSMA); GD2, carcinoembryonic antigen (CEA); melanoma antigen a/melanoma antigen recognized by T cells 1 (MelanA/MART1); Ras; gp100, Proteinase 3 (PR1), Bcr-abl; Survivin; prostate specific antigen (PSA); human telomerase reverse transcriptase (hTERT); EphA2; ML-IAP; alphafetoprotein (AFP); EpCAM; ERG; NA17; PAX3; ALK; Androgen receptor (AR); Cyclin B1; MYCN; RhoC; tyrosine related protein 2 (TRP-2); GD3; Fucosyl GM1; PSCA; sLe(a); CYP1B1; PLCA1; GM3; BORIS; Tn; GloboH; Ets variant gene 6/acute myeloid leukemia 1 gene ETS (ETV6-AML); NY-BR-1; RGS5; squamous antigen rejecting tumor or 3 (SART3); STn; Carbonic anhydrase IX; PAX5; OY-TES1; Sperm protein 17; LCK; HMWMAA; AKAP-4; SSX2; B7H3; Legumain; Tie 2; Page4; VEGFR2; MAD-CT-1; FAP; PDGFR; MAD-CT-2; Fosrelated antigen 1; TAG-72; 9D7; EphA3; Telomerase; SAP-1; BAGE family; CAGE family; GAGE family; MAGE family; SAGE family; XAGE family; preferentially expressed antigen of melanoma (PRAME); melanocortin 1 receptor (MC1R); β-catenin; BRCA1/2; CDK4; chronic myelogenous leukemia 66 (CML66); TGF-β. In certain embodiments, the host self-antigens include prostatic acidic phosphatase, Wilms tumor suppressor protein, mesothelin, or Her-2.

In some embodiments the tumor antigen is derived from a cancer. The cancer includes, but is not limited to, Acute lymphoblastic leukemia; Acute myeloid leukemia; Adrenocortical carcinoma; AIDS-related cancers; AIDS-related lymphoma; Anal cancer; Appendix cancer; Astrocytoma, childhood cerebellar or cerebral; Basal cell carcinoma; Bile duct cancer, extrahepatic; Bladder cancer; Bone cancer, Osteosarcoma/Malignant fibrous histiocytoma; Brainstem glioma; Brain tumor; Brain tumor, cerebellar astrocytoma; Brain tumor, cerebral astrocytoma/malignant glioma; Brain tumor, ependymoma; Brain tumor, medulloblastoma; Brain tumor, supratentorial primitive neuroectodermal tumors; Brain tumor, visual pathway and hypothalamic glioma; Breast cancer; Bronchial adenomas/carcinoids; Burkitt lymphoma; Carcinoid tumor, childhood; Carcinoid tumor, gastrointestinal; Carcinoma of unknown primary; Central nervous system lymphoma, primary; Cerebellar astrocytoma, childhood; Cerebral astrocytoma/Malignant glioma, childhood; Cervical cancer; Childhood cancers; Chronic lymphocytic leukemia; Chronic myelogenous leukemia; Chronic myeloproliferative disorders; Colon Cancer; Cutaneous T-cell lymphoma; Desmoplastic small round cell tumor; Endometrial cancer; Ependymoma; Esophageal cancer; Ewing's sarcoma in the Ewing family of tumors; Extracranial germ cell tumor, Childhood; Extragonadal Germ cell tumor; Extrahepatic bile duct cancer; Eye Cancer, Intraocular melanoma; Eye Cancer, Retinoblastoma; Gallbladder cancer; Gastric (Stomach) cancer; Gastrointestinal Carcinoid Tumor; Gastrointestinal stromal tumor (GIST); Germ cell tumor: extracranial, extragonadal, or ovarian; Gestational trophoblastic tumor; Glioma of the brain stem; Glioma, Childhood Cerebral Astrocytoma; Glioma, Childhood Visual Pathway and Hypothalamic; Gastric carcinoid; Hairy cell leukemia; Head and neck cancer; Heart cancer; Hepatocellular (liver) cancer; Hodgkin lymphoma; Hypopharyngeal cancer; Hypothalamic and visual pathway glioma, childhood; Intraocular Melanoma; Islet Cell Carcinoma (Endocrine Pancreas); Kaposi sarcoma; Kidney cancer (renal cell cancer); Laryngeal Cancer; Leukemias; Leukemia, acute lymphoblastic (also called acute lymphocytic leukemia); Leukemia, acute myeloid (also called acute myelogenous leukemia); Leukemia, chronic lymphocytic (also called chronic lymphocytic leukemia); Leukemia, chronic myelogenous (also called chronic myeloid leukemia); Leukemia, hairy cell; Lip and Oral Cavity Cancer; Liver Cancer (Primary); Lung Cancer, Non-Small Cell; Lung Cancer, Small Cell; Lymphomas; Lymphoma, AIDS-related; Lymphoma, Burkitt; Lymphoma, cutaneous T-Cell; Lymphoma, Hodgkin; Lymphomas, Non-Hodgkin (an old classification of all lymphomas except Hodgkin's); Lymphoma, Primary Central Nervous System; Marcus Whittle, Deadly Disease; Macroglobulinemia, Waldenstrim; Malignant Fibrous Histiocytoma of Bone/Osteosarcoma; Medulloblastoma, Childhood; Melanoma; Melanoma, Intraocular (Eye); Merkel Cell Carcinoma; Mesothelioma, Adult Malignant; Mesothelioma, Childhood; Metastatic Squamous Neck Cancer with Occult Primary; Mouth Cancer; Multiple Endocrine Neoplasia Syndrome, Childhood; Multiple Myeloma/Plasma Cell Neoplasm; Mycosis Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple (Cancer of the Bone-Marrow); Myeloproliferative Disorders, Chronic; Nasal cavity and paranasal sinus cancer; Nasopharyngeal carcinoma; Neuroblastoma; Non-Hodgkin lymphoma; Non-small cell lung cancer; Oral Cancer; Oropharyngeal cancer; Osteosarcoma/malignant fibrous histiocytoma of bone; Ovarian cancer; Ovarian epithelial cancer (Surface epithelial-stromal tumor); Ovarian germ cell tumor; Ovarian low malignant potential tumor; Pancreatic cancer; Pancreatic cancer, islet cell; Paranasal sinus and nasal cavity cancer; Parathyroid cancer; Penile cancer; Pharyngeal cancer; Pheochromocytoma; Pineal astrocytoma; Pineal germinoma; Pineoblastoma and supratentorial primitive neuroectodermal tumors, childhood; Pituitary adenoma; Plasma cell neoplasia/Multiple myeloma; Pleuropulmonary blastoma; Primary central nervous system lymphoma; Prostate cancer; Rectal cancer; Renal cell carcinoma (kidney cancer); Renal pelvis and ureter, transitional cell cancer; Retinoblastoma; Rhabdomyosarcoma, childhood; Salivary gland cancer; Sarcoma, Ewing family of tumors; Sarcoma, Kaposi; Sarcoma, soft tissue; Sarcoma, uterine; Sezary syndrome; Skin cancer (nonmelanoma); Skin cancer (melanoma); Skin carcinoma, Merkel cell; Small cell lung cancer; Small intestine cancer; Soft tissue sarcoma; Squamous cell carcinoma—see Skin cancer (nonmelanoma); Squamous neck cancer with occult primary, metastatic; Stomach cancer; Supratentorial primitive neuroectodermal tumor, childhood; T-Cell lymphoma, cutaneous (Mycosis Fungoides and Sezary syndrome); Testicular cancer; Throat cancer; Thymoma, childhood; Thymoma and Thymic carcinoma; Thyroid cancer; Thyroid cancer, childhood; Transitional cell cancer of the renal pelvis and ureter; Trophoblastic tumor, gestational; Unknown primary site, carcinoma of, adult; Unknown primary site, cancer of, childhood; Ureter and renal pelvis, transitional cell cancer; Urethral cancer; Uterine cancer, endometrial; Uterine sarcoma; Vaginal cancer; Visual pathway and hypothalamic glioma, childhood; Vulvar cancer; Waldenstrom macroglobulinemia; and Wilms tumor (kidney cancer.)

In some embodiments, the pathogen specific antigen is a MHC-E supertope. In some embodiments, the MHC-E supertope is a HIV epitope. In some embodiments, the MHC-E supertope is at least 10%, at least 20%, at least 30%, at least 40%, at least 50%, at least 60%, at least 70%, at least 80%, at least 85%, at least 90%, at least 95%, or 100%<identical to

(SEQ ID NO: 13)
LDAWEKIRLRPGGKK;
(SEQ ID NO: 14)
DAWEKIRLR;
(SEQ ID NO: 15)
KKAQQAAADTGNSSQ;
(SEQ ID NO: 16)
KAQQAAADT;
(SEQ ID NO: 17)
QMVHQAISPRTLNAW;
(SEQ ID NO: 18)
HQAISPRTL;
(SEQ ID NO: 19)
NTMLNTVGGHQAAMQ;
(SEQ ID NO: 20)
VGGHQAAMQ;
(SEQ ID NO: 21)
STLQEQIGWMTNNPP;
(SEQ ID NO: 22)
STLQEQIGW;
(SEQ ID NO: 23)
IVRMYSPVSILDIRQ;
(SEQ ID NO: 24)
RMYSPVSIL;
(SEQ ID NO: 25)
QKQEPIDKELYPLAS;
(SEQ ID NO: 26)
KQEPIDKEL;
(SEQ ID NO: 27)
SFSFPQITLWQRPLV;
(SEQ ID NO: 28)
VRQYDQILIEICGKK;
(SEQ ID NO: 29)
EPFRKQNPDIVIYQL;
(SEQ ID NO: 30)
YVDGAANRETKLGKA;
(SEQ ID NO: 31)
EEHEKYSNWRAMAS;
or
(SEQ ID NO: 32)
ILDLWVYHTQGYFPD.

In some embodiments, one or more of the MHC-E supertopes are used to generate a fusion protein. The fusion protein may contain one or more of the MHC-E supertopes, in any order.

In some embodiments, the HCMV vector is administered in an amount effective to elicit a CD8+ T cell response to the at least one heterologous antigen. In some embodiments, the CD8+ T cell response elicited by the vector is characterized by having at least 10% of the CD8+ T cells directed against epitopes presented by MHC-E. In further examples, at least 15%, at least 20%, at least 30%, at least 40%, at least 50%, at least 60%, at least 70%, at least 75%, at least 80%, at least 85%, at least 90%, or at least 95% of the CD8+ T cells are restricted by MHC-E. In some embodiments, the CD8+ T cells restricted by MHC-E recognized peptides shared by at least 90% of other subjects immunized with the vector. In some embodiments, the CD8+ T cells are directed against a supertope presented by MHC-E.

In some embodiments, the method further comprises identifying a CD8+ T cell receptor (TCR) from the CD8+ T cells elicited from the HCMV vector.

The TCR can be identified by DNA or RNA sequencing. In some embodiments, the CD8+ TCR recognizes a MHC-E/heterologous antigen-derived peptide complex. In some embodiments, the CD8+ TCR recognizes MHC-E supertopes. In some embodiments, the MHC-E supertope is a human immunodeficiency virus epitope. In some embodiments, the MHC-E supertope is at least 10%, at least 20%, at least 30%, at least 40%, at least 50%, at least 60%, at least 70%, at least 80%, at least 85%, at least 90%, at least 95%, or 100% identical to

(SEQ ID NO: 13)
LDAWEKIRLRPGGKK;
(SEQ ID NO: 14)
DAWEKIRLR;
(SEQ ID NO: 15)
KKAQQAAADTGNSSQ;
(SEQ ID NO: 16)
KAQQAAADT;
(SEQ ID NO: 17)
QMVHQAISPRTLNAW;
(SEQ ID NO: 18)
HQAISPRTL;
(SEQ ID NO: 19)
NTMLNTVGGHQAAMQ;
(SEQ ID NO: 20)
VGGHQAAMQ;
(SEQ ID NO: 21)
STLQEQIGWMTNNPP;
(SEQ ID NO: 22)
STLQEQIGW;
(SEQ ID NO: 23)
IVRMYSPVSILDIRQ;
(SEQ ID NO: 24)
RMYSPVSIL;
(SEQ ID NO: 25)
QKQEPIDKELYPLAS;
(SEQ ID NO: 26)
KQEPIDKEL;
(SEQ ID NO: 27)
SFSFPQITLWQRPLV;
(SEQ ID NO: 28)
VRQYDQILIEICGKK;
(SEQ ID NO: 29)
EPFRKQNPDIVIYQL;
(SEQ ID NO: 30)
YVDGAANRETKLGKA;
(SEQ ID NO: 31)
EEHEKYSNWRAMAS;
or
(SEQ ID NO: 32)
ILDLWVYHTQGYFPD.

In some embodiments, the method further comprises the use of supertope peptides to identify a MHC-E restricted CD8+ T cell receptor (TCR) from CD8+ T cells elicited by a non-human primate CMV, such as rhesus or cynomolgus macaque CMV (RhCMV or CyCMV), that is defective in expression of orthologs of UL128, UL130, UL146 and UL147 (and optionally UL82) and expresses orthologues of UL40 and US28. MHC-E restricted CD+ T cells would be elicited in rhesus macaques with RhCMV or in cynomolgus macaques with CyCMV.

In some embodiments, the CD8+ T cell response elicited by the HCMV vector is characterized by having at least 10% of the CD8+ T cells directed against epitopes presented by MHC-II. In further examples, at least 15%, at least 20%, at least 30%, at least 40%, at least 50%, at least 60%, at least 70%, at least 75%, at least 80%, at least 85%, at least 90%, or at least 95% of the CD8+ T cells are restricted by MHC-II. In some embodiments, the CD8+ T cells restricted by MHC-II recognized peptides shared by at least 90% of other subjects immunized with the vector. In some embodiments, the CD8+ T cells are directed against a supertope presented by MHC-II.

In some embodiments, the method further comprises identifying a CD8+ T cell receptor (TCR) from the CD8+ T cells elicited from the HCMV vector. The TCR can be identified by DNA or RNA sequencing. In some embodiments, the CD8+ TCR recognizes a MHC-II/heterologous antigen-derived peptide complex. In some embodiments, the CD8+ TCR recognizes MHC-II supertopes.

In some embodiments, the CD8+ T cell response elicited by a UL18-deficient HCMV vector that also lacks US11 is characterized by having at least 10% of the CD8+ T cells directed against epitopes presented by MHC-Ia. In further examples, at least 15%, at least 20%, at least 30%, at least 40%, at least 50%, at least 60%, at least 75%, at least 90%, at least 95% or at least 95% of the CD8+ T cells are restricted by MHC-Ia.

In some embodiments, the method further comprises identifying a CD8+ T cell receptor (TCR) from the CD8+ T cells elicited from the UL18 and US11-deficient HCMV vector. The TCR can be identified by DNA or RNA sequencing. In some embodiments, the CD8+ TCR recognizes a MHC-Ia/heterologous antigen-derived peptide complex.

Also disclosed herein is a method of generating CD8+ T cells that recognize MHC-E peptide complexes. This method involves administering to a first subject a HCMV vector in an amount effective to generate a set of CD8+ T cells that recognize MHC-E/peptide complexes. The CMV vector comprises a first nucleic acid sequence encoding at least one heterologous antigen and does not express: an UL18 protein, an UL128 protein, an UL130 protein, an UL146 protein, and an UL147 protein. The vector might also lack an UL82 protein. In some embodiments, the HCMV vector expresses UL40 and US28. In some embodiments, the HCMV vector does not express an UL18, UL138, UL130, UL146, and UL147 protein and comprises a nucleic acid sequence encoding UL40, US28, and a microRNA (miRNA) recognition element (MRE). In some embodiments, the MRE contains target sites for microRNAs expressed in endothelial cells. Examples of such miRNAs expressed in endothelial cells are miR126, miR-126-3p, miR-130a, miR-210, miR-221/222, miR-378, miR-296, and miR-328.

The antigen may be any antigen, including a pathogen-specific antigen, a tumor virus antigen, a tumor antigen, or a host self-antigen. In some embodiments, the host self-antigen is an antigen derived from the variable region of a T cell receptor or a B cell receptor.

This method further comprises: identifying a first CD8+ T cell receptor from the set of CD8+ T cells, wherein the first CD8+ T cell receptor recognizes a MHC-E/heterologous antigen-derived peptide complex. In some embodiments, the first CD8+ T cell receptor is identified by DNA or RNA sequencing. In some embodiments, this method may further comprise transfecting the one or more CD8+ T cells with an expression vector, wherein the expression vector comprises a nucleic acid sequence encoding a second CD8+ T cell receptor and a promoter operably linked to the nucleic acid sequence encoding the T cell receptor, wherein the second CD8+ T cell receptor comprises CDR3α and CDR3β of the first CD8+ T cell receptor, thereby generating one or more transfected CD8+ T cells that recognize a MHC-E/heterologous antigen-derived peptide complex. The one or more CD8+ T cells for transfection with the expression vector may be isolated from the first subject or a second subject.

In some embodiments, the method further comprises identifying a CD8+ T cell receptor from the CD8+ T cells elicited by the HCMV vector, wherein the CD8+ T cell receptor recognizes a MHC-E/heterologous antigen-derived peptide complex. In some embodiments, the method further comprises identifying a MHC-E restricted CD8+ T cell receptor from CD8+ T cells elicited by a non-human primate CMV, such as rhesus or cynomolgus macaque CMV (RhCMV or CyCMV), that is defective in expression of orthologs of UL128, UL130, UL146, and UL147 and expresses orthologues of UL40 and US28. MHC-E restricted CD8+ T cells would be elicited in rhesus macaques with RhCMV or in cynomolgus macaques with CyCMV. In some embodiments, the CD8+ T cell receptor is identified by RNA or DNA sequencing. In some embodiments, the method further comprises a CD8+ T cell receptor that recognizes MHC-E supertopes. In some embodiments, the MHC-E supertope is a human immunodeficiency virus epitope. In some embodiments, the MHC-E supertope is at least 10%, at least 20%, at least 30%, at least 40%, at least 50%, at least 60%, at least 70%, at least 80%, at least 85%, at least 90%, at least 95%, or 100% identical to

(SEQ ID NO: 13)
LDAWEKIRLRPGGKK;
(SEQ ID NO: 14)
DAWEKIRLR;
(SEQ ID NO: 15)
KKAQQAAADTGNSSQ;
(SEQ ID NO: 16)
KAQQAAADT;
(SEQ ID NO: 17)
QMVHQAISPRTLNAW;
(SEQ ID NO: 18)
HQAISPRTL;
(SEQ ID NO: 19)
NTMLNTVGGHQAAMQ;
(SEQ ID NO: 20)
VGGHQAAMQ;
(SEQ ID NO: 21)
STLQEQIGWMTNNPP;
(SEQ ID NO: 22)
STLQEQIGW;
(SEQ ID NO: 23)
IVRMYSPVSILDIRQ;
(SEQ ID NO: 24)
RMYSPVSIL;
(SEQ ID NO: 25)
QKQEPIDKELYPLAS;
(SEQ ID NO: 26)
KQEPIDKEL;
(SEQ ID NO: 27)
SFSFPQITLWQRPLV;
(SEQ ID NO: 28)
VRQYDQILIEICGKK;
(SEQ ID NO: 29)
EPFRKQNPDIVIYQL;
(SEQ ID NO: 30)
YVDGAANRETKLGKA;
(SEQ ID NO: 31)
EEHEKYSNWRAMAS;
or
(SEQ ID NO: 32)
ILDLWVYHTQGYFPD.

Also disclosed here in is a method of generating TCR-transgenic CD8+ T cells that recognize MHC-E-peptide complexes, the method comprising: (a) identifying a first CD8+ TCR from a set of CD8+ T cells, wherein the set of CD8+ T cells are generated from the recombinant HCMV vector, wherein the first CD8+ TCR recognizes a MHC-E/heterologous antigen-derived peptide complex; (b) isolating one or more CD8+ T cells from a second subject; and (c) transfecting the one or more CD8+ T cells with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3α and CDR3β of the first CD8+ TCR, thereby generating one or more TCR-transgenic CD8+ T cells that recognize MHC-E-peptide complexes.

Also disclosed is a TCR-transfected CD8+ T cell that recognizes MHC-E-peptide complexes prepared by a process comprising the steps of: (1) administering to a first subject a HCMV vector (deleted for UL18, UL128, UL130, UL146, UL147, and, in some embodiments, UL82; expressing UL40 and US28; and, in some embodiments, expressing a nucleic acid sequence encoding a microRNA recognition element) in an amount effective to generate a set of CD8+ T cells that recognize MHC-E/peptide complexes, wherein the recombinant HCMV vector comprises at least one heterologous antigen; (2) identifying a first CD8+ T cell receptor from the set of CD8+ T cells, wherein the first CD8+ T cell receptor recognizes a MHC-E/heterologous antigen-derived peptide complex; (3) isolating one or more CD8+ T cells from the first subject or a second subject; and (4) transfecting the one or more CD8+ T cells isolated from the first or second subject with an expression vector, thereby creating a transfected T cell that recognizes MHC-E peptide complexes wherein the transfected CD8+ T cells generate an immune response to the MHC-E/heterologous antigen-derive peptide complex.

In some embodiments, this method may further comprise transfecting the one or more CD8+ T cells with an expression vector, wherein the expression vector comprises a nucleic acid sequence encoding a second CD8+ T cell receptor and a promoter operably linked to the nucleic acid sequence encoding the T cell receptor, wherein the second CD8+ T cell receptor comprises CDR3α and CDR3β of the first CD8+ T cell receptor, thereby generating one or more transected CD8+ T cells that recognize a MHC-E/heterologous antigen-derived peptide complex. The one or more CD8+ T cells for transfection with the expression vector may be isolated from the first subject or a second subject.

In some embodiments, the first and/or second CD8+ T cell receptors are identified by RNA or DNA sequencing. In some embodiments, the first and/or second CD8+ T cell receptor recognizes MHC-E supertopes. In some embodiments, the MHC-E supertope is a human immunodeficiency virus epitope. In some embodiments, the MHC-E supertope is at least 10%, at least 20%, at least 30%, at least 40%, at least 50%, at least 60%, at least 70%, at least 80%, at least 85%, at least 90%, at least 95%, or 100% identical to

(SEQ ID NO: 13)
LDAWEKIRLRPGGKK;
(SEQ ID NO: 14)
DAWEKIRLR;
(SEQ ID NO: 15)
KKAQQAAADTGNSSQ;
(SEQ ID NO: 16)
KAQQAAADT;
(SEQ ID NO: 17)
QMVHQAISPRTLNAW;
(SEQ ID NO: 18)
HQAISPRTL;
(SEQ ID NO: 19)
NTMLNTVGGHQAAMQ;
(SEQ ID NO: 20)
VGGHQAAMQ;
(SEQ ID NO: 21)
STLQEQIGWMTNNPP;
(SEQ ID NO: 22)
STLQEQIGW;
(SEQ ID NO: 23)
IVRMYSPVSILDIRQ;
(SEQ ID NO: 24)
RMYSPVSIL;
(SEQ ID NO: 25)
QKQEPIDKELYPLAS;
(SEQ ID NO: 26)
KQEPIDKEL;
(SEQ ID NO: 27)
SFSFPQITLWQRPLV;
(SEQ ID NO: 28)
VRQYDQILIEICGKK;
(SEQ ID NO: 29)
EPFRKQNPDIVIYQL;
(SEQ ID NO: 30)
YVDGAANRETKLGKA;
(SEQ ID NO: 31)
EEHEKYSNWRAMAS;
or
(SEQ ID NO: 32)
ILDLWVYHTQGYFPD.

In some embodiments, the nucleic acid sequence encoding the second CD8+ TCR is identical to the nucleic acid sequence encoding the first CD8+ TCR. In some embodiments, the first and/or second subject is a human or nonhuman primate. In some embodiments, the second CD8+ TCR is a chimeric CD8+ TCR. In some embodiments, the first subject is a nonhuman primate and the second subject is a human, and wherein the second CD8+ TCR is a chimeric nonhuman primate-human CD8+ TCR comprising the non-human primate CDR3α and CDR3β of the first CD8+ TCR. In some embodiments, the second CD8+ TCR comprises the nonhuman primate CDR1α, CDR2α, CDR3α, CDR1β, CDR2β, and CDR3β of the first CD8+ TCR. In some embodiments, the second CD8+ TCR comprises the CDR1α, CDR2α, CDR3α, CDR1β, CDR2β, and CDR3β of the first CD8+ TCR.

Also disclosed herein are methods of treating a disease, such as cancer, a pathogenic infection, or an immune disease or disorder, the method comprising administering the transfected T cell that recognizes MHC-E peptide complexes to the first or second subject. Also disclosed herein are methods of inducing an immune response to a host self-antigen or tissue-specific antigen, the method comprising administering the transfected T cell that recognizes MHC-E peptide complexes to the first or second subject.

The cancer, includes but is not limited to, acute myelogenous leukemia, chronic myelogenous leukemia, myelodysplastic syndrome, acute lymphoblastic leukemia, chronic lymphoblastic leukemia, acute lymphoblastic leukemia, non-Hodgkin's lymphoma, multiple myeloma, malignant melanoma, mesothelioma, malignant mesothelioma, kidney cancer, cervical cancer, oropharyngeal cancer, anal cancer, penile cancer, vaginal cancer, vulvar cancer, breast cancer, lung cancer, ovarian cancer, prostate cancer, pancreatic cancer, colon cancer, renal cell carcinoma, and germ cell tumors.

The pathogenic infection, includes but is not limited to, human immunodeficiency virus, herpes simplex virus type I, herpes simplex virus type 2, hepatitis B virus, hepatitis C virus, papillomavirus, Plasmodium parasites, and Mycobacterium tuberculosis.

Also disclosed here are methods of generating CD8+ T cells that recognize MHC-E peptide complexes, the method comprising: (a) identifying a first CD8+ TCR that recognizes a MHC-E/supertope peptide complex from a set of CD8+ T cells that recognize MHC-E in complex with the HIV supertope peptides, wherein the set of CD8+ T cells are generated from a recombinant rhesus (RhCMV) or cynomolgus CMV (CyCCMV) vector deficient for orthologs of UL128, UL130, UL146, and UL147 and expressing HIV antigens in an amount effective to generate the set of CD8+ T cells; (b) isolating one or more CD8+ T cells from a second subject; and (c) transfecting the one or more CD8+ T cells with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3α and CDR3β of the first CD8+ TCR, thereby generating one or more CD8+ T cells that recognize MHC-E peptide complexes.

Also disclosed herein is a method of generating CD8+ T cells that recognize MHC-II peptide complexes. This method involves administering to a first subject (or animal) a CMV vector in an amount effective to generate a set of CD8+ T cells that recognize MHC-II/peptide complexes. The CMV vector comprises a first nucleic acid sequence encoding at least one heterologous antigen and does not express: an UL18 protein, an UL128 protein, an UL130 protein, an UL146 protein, or an UL147 protein, and, in some embodiments, an UL82 protein.

In some embodiments, the UL18-deficient HCMV vector also comprises a nucleic acid sequence encoding a microRNA (miRNA) recognition element (MRE). In some embodiments, the MRE contains target sites for microRNAs expressed in myeloid cells. Examples of such miRNAs expressed in myeloid cells are miR-142-ep, miR-223, miR-27a, miR-652, miR-155, miR-146a, miR-132, miR-21, and miR-125.

The antigen may be any antigen, including a pathogen-specific antigen, a tumor virus antigen, a tumor antigen, or a host self-antigen. In some embodiments, the host self-antigen is an antigen derived from the variable region of a T cell receptor or a B cell receptor.

This method further comprises: identifying a first CD8+ T cell receptor from the set of CD8+ T cells, wherein the first CD8+ T cell receptor recognizes a MHC-II/heterologous antigen-derived peptide complex. In some embodiments, the first CD8+ T cell receptor is identified by DNA or RNA sequencing. In some embodiments, this method may further comprise transfecting the one or more CD8+ T cells with an expression vector, wherein the expression vector comprises a nucleic acid sequence encoding a second CD8+ T cell receptor and a promoter operably linked to the nucleic acid sequence encoding the T cell receptor, wherein the second CD8+ T cell receptor comprises CDR3α and CDR3β of the first CD8+ T cell receptor, thereby generating one or more transfected CD8+ T cells that recognize a MHC-II/heterologous antigen-derived peptide complex. The one or more CD8+ T cells for transfection with the expression vector may be isolated from the first subject or a second subject.

In some embodiments, the method further comprises identifying a CD8+ T cell receptor from the CD8+ T cells elicited by the HCMV vector, wherein the CD8+ T cell receptor recognizes a MHC-II/heterologous antigen-derived peptide complex. In some embodiments, the CD8+ T cell receptor is identified by RNA or DNA sequencing. In some embodiments, the method further comprises a CD8+ T cell receptor that recognizes supertopes.

Also disclosed are methods of generating CD8+ T cells that recognize MHC-II-peptide complexes, the method comprising: (a) identifying a first CD8+ TCR that recognizes a MHC-II/heterologous antigen-derived peptide complex from a set of CD8+ T cells that recognize MHC-II/peptide complexes, wherein the set of CD8+ T cells are generated from the recombinant HCMV vector; (b) isolating one or more CD8+ T cells from a second subject; and (c) transfecting the one or more CD8+ T cells with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3α and CDR3β of the first CD8+ TCR, thereby generating one or more CD8+ T cells that recognize a MHC-II peptide complexes.

Also disclosed is a TCR-transfected CD8+ T cell that recognizes MHC-II-peptide complexes prepared by a process comprising the steps of: (1) administering to a first subject a UL18-deficient HCMV vector (also deleted for UL128, UL130, UL146, or UL147 (or combinations thereof), and, in some embodiments UL82; and/or expressing a nucleic acid encoding a microRNA recognition element) in an amount effective to generate a set of CD8+ T cells that recognize MHC-II/peptide complexes, wherein the recombinant CMV vector comprises at least one heterologous antigen; (2) identifying a first CD8+ T cell receptor from the set of CD8+ T cells, wherein the first CD8+ T cell receptor recognizes a MHC-II/heterologous antigen-derived peptide complex; (3) isolating one or more CD8+ T cells from the first subject or a second subject; and (4) transfecting the one or more CD8+ T cells isolated from the first or second subject with an expression vector, thereby creating a transfected T cell that recognizes MHC-II peptide complexes wherein the transfected CD8+ T cells generate an immune response to the MHC-Wheterologous antigen-derive peptide complex.

In some embodiments, this method may further comprise transfecting the one or more CD8+ T cells with an expression vector, wherein the expression vector comprises a nucleic acid sequence encoding a second CD8+ T cell receptor and a promoter operably linked to the nucleic acid sequence encoding the T cell receptor, wherein the second CD8+ T cell receptor comprises CDR3α and CDR3β of the first CD8+ T cell receptor, thereby generating one or more transected CD8+ T cells that recognize a MHC-II/heterologous antigen-derived peptide complex. The one or more CD8+ T cells for transfection with the expression vector may be isolated from the first subject or a second subject.

In some embodiments, the first and/or second CD8+ T cell receptors are identified by RNA or DNA sequencing. In some embodiments, the first and/or second CD8+ T cell receptor recognizes MHC-II supertopes.

In some embodiments, the nucleic acid sequence encoding the second CD8+ TCR is identical to the nucleic acid sequence encoding the first CD8+ TCR. In some embodiments, the first and/or second subject is a human or nonhuman primate. In some embodiments, the second CD8+ TCR is a chimeric CD8+ TCR. In some embodiments, the first subject is a nonhuman primate and the second subject is a human, and wherein the second CD8+ TCR is a chimeric nonhuman primate-human CD8+ TCR comprising the non-human primate CDR3α and CDR3β of the first CD8+ TCR. In some embodiments, the second CD8+ TCR comprises the nonhuman primate CDR1α, CDR2α, CDR3α, CDR1β, CDR2β, and CDR3β of the first CD8+ TCR. In some embodiments, the second CD8+ TCR comprises the CDR1α, CDR2α, CDR3α, CDR1β, CDR2β, and CDR3p of the first CD8+ TCR.

Also disclosed herein are methods of treating a disease, such as cancer, a pathogenic infection, or an immune disease or disorder, the method comprising administering the transfected T cell that recognizes MHC-II peptide complexes to the first or second subject. Also disclosed herein are methods of inducing an immune response to a host self-antigen or tissue-specific antigen, the method comprising administering the transfected T cell that recognizes MHC-II peptide complexes to the first or second subject.

The cancer, includes but is not limited to, acute myelogenous leukemia, chronic myelogenous leukemia, myelodysplastic syndrome, acute lymphoblastic leukemia, chronic lymphoblastic leukemia, acute lymphoblastic leukemia, non-Hodgkin's lymphoma, multiple myeloma, malignant melanoma, mesothelioma, malignant mesothelioma, kidney cancer, cervical cancer, oropharyngeal cancer, anal cancer, penile cancer, vaginal cancer, vulvar cancer, breast cancer, lung cancer, ovarian cancer, prostate cancer, pancreatic cancer, colon cancer, renal cell carcinoma, and germ cell tumors.

The pathogenic infection, includes but is not limited to, human immunodeficiency virus, herpes simplex virus type I, herpes simplex virus type 2, hepatitis B virus, hepatitis C virus, papillomavirus, Plasmodium parasites, and Mycobacterium tuberculosis.

Also disclosed herein is a method of generating CD8+ T cells that recognize MHC-Ia peptide complexes. This method involves administering to a first subject a UL18-deficient CMV vector that also lacks an US11 protein in an amount effective to generate a set of CD8+ T cells that recognize MHC-Ia/peptide complexes. The CMV vector comprises a first nucleic acid sequence encoding at least one heterologous antigen and does not express: an US11 protein and an UL18 protein. The vector might also lack an UL128 protein, an UL130 protein, or an UL146 protein, an UL147 protein, and/or an UL82 protein. The antigen may be any antigen, including a pathogen-specific antigen, a tumor virus antigen, a tumor antigen, or a host self-antigen. In some embodiments, the host self-antigen is an antigen derived from the variable region of a T cell receptor or a B cell receptor.

This method further comprises: identifying a first CD8+ T cell receptor from the set of CD8+ T cells, wherein the first CD8+ T cell receptor recognizes a MHC-Ia/heterologous antigen-derived peptide complex. In some embodiments, the first CD8+ T cell receptor is identified by DNA or RNA sequencing. In some embodiments, this method may further comprise transfecting the one or more CD8+ T cells with an expression vector, wherein the expression vector comprises a nucleic acid sequence encoding a second CD8+ T cell receptor and a promoter operably linked to the nucleic acid sequence encoding the T cell receptor, wherein the second CD8+ T cell receptor comprises CDR3α and CDR3β of the first CD8+ T cell receptor, thereby generating one or more transfected CD8+ T cells that recognize a MHC-Ia/heterologous antigen-derived peptide complex. The one or more CD8+ T cells for transfection with the expression vector may be isolated from the first subject or a second subject.

In some embodiments, the method further comprises identifying a CD8+ T cell receptor from the CD8+ T cells elicited by the CMV vector, wherein the CD8+ T cell receptor recognizes a MHC-Ia/heterologous antigen-derived peptide complex. In some embodiments, the CD8+ T cell receptor is identified by RNA or DNA sequencing.

Also disclosed are methods of generating CD8+ T cells that recognize MHC-I-peptide complexes, the method comprising: (a) identifying a first CD8+ TCR that recognizes a MHC-I/heterologous antigen-derived peptide complex from a set of CD8+ T cells that recognize a MHC-I/heterologous antigen-derived peptide complex, wherein the set of CD8+ T cells are generated from the recombinant HCMV vector; (b) isolating one or more CD8+ T cells from a second subject; and (c) transfecting the one or more CD8+ T cells with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3α and CDR3β of the first CD8+ TCR, thereby generating one or more CD8+ T cells that recognize MHC-I peptide complexes.

Also disclosed is a transfected CD8+ T cell that recognizes MHC-Ia-peptide complexes prepared by a process comprising the steps of: (1) administering to a first subject a CMV vector defective for US11 and UL18 (additionally the vector might be defective for UL128, UL130, UL146, UL147, and/or UL82; expressing UL40 and/or US28) in an amount effective to generate a set of CD8+ T cells that recognize MHC-Ia/peptide complexes, wherein the recombinant CMV vector comprises at least one heterologous antigen; (2) identifying a first CD8+ T cell receptor from the set of CD8+ T cells, wherein the first CD8+ T cell receptor recognizes a MHC-Ia/heterologous antigen-derived peptide complex; (3) isolating one or more CD8+ T cells from the first subject or a second subject; and (4) transfecting the one or more CD8+ T cells isolated from the first or second subject with an expression vector, thereby creating a transfected T cell that recognizes MHC-Ia peptide complexes wherein the transfected CD8+ T cells generate an immune response to the MHC-Ia/heterologous antigen-derive peptide complex.

In some embodiments, this method may further comprise transfecting the one or more CD8+ T cells with an expression vector, wherein the expression vector comprises a nucleic acid sequence encoding a second CD8+ T cell receptor and a promoter operably linked to the nucleic acid sequence encoding the T cell receptor, wherein the second CD8+ T cell receptor comprises CDR3α and CDR3β of the first CD8+ T cell receptor, thereby generating one or more transected CD8+ T cells that recognize a MHC-Ia/heterologous antigen-derived peptide complex. The one or more CD8+ T cells for transfection with the expression vector may be isolated from the first subject or a second subject.

In some embodiments, the first and/or second CD8+ T cell receptors are identified by RNA or DNA sequencing.

In some embodiments, the nucleic acid sequence encoding the second CD8+ TCR is identical to the nucleic acid sequence encoding the first CD8+ TCR. In some embodiments, the second CD8+ TCR is a chimeric CD8+ TCR. In some embodiments, the second CD8+ TCR comprises the CDR1α, CDR2α, CDR3α, CDR1β, CDR2β, and CDR3p of the first CD8+ TCR.

Also disclosed herein are methods of treating a disease, such as cancer, a pathogenic infection, or an immune disease or disorder, the method comprising administering the transfected T cell that recognizes MHC-Ia peptide complexes to the first or second subject. Also disclosed herein are methods of inducing an immune response to a host self-antigen or tissue-specific antigen, the method comprising administering the transfected T cell that recognizes MHC-Ia peptide complexes to the first or second subject.

The cancer, includes but is not limited to, acute myelogenous leukemia, chronic myelogenous leukemia, myelodysplastic syndrome, acute lymphoblastic leukemia, chronic lymphoblastic leukemia, acute lymphoblastic leukemia, non-Hodgkin's lymphoma, multiple myeloma, malignant melanoma, mesothelioma, malignant mesothelioma, kidney cancer, cervical cancer, oropharyngeal cancer, anal cancer, penile cancer, vaginal cancer, vulvar cancer, breast cancer, lung cancer, ovarian cancer, prostate cancer, pancreatic cancer, colon cancer, renal cell carcinoma, and germ cell tumors.

The pathogenic infection, includes but is not limited to, human immunodeficiency virus, herpes simplex virus type I, herpes simplex virus type 2, hepatitis B virus, hepatitis C virus, papillomavirus, Plasmodium parasites, and Mycobacterium tuberculosis.

### III. HIV Supertope Constructs

Also disclosed are human immunodeficiency virus antigens between 9 and 15 amino acids in length and that is at least 90%, at least 95%, or 100% identical to the amino acid sequence of

(SEQ ID NO: 13)
LDAWEKIRLRPGGKK;
(SEQ ID NO: 14)
DAWEKIRLR;
(SEQ ID NO: 15)
KKAQQAAADTGNSSQ;
(SEQ ID NO: 16)
KAQQAAADT;
(SEQ ID NO: 17)
QMVHQAISPRTLNAW;
(SEQ ID NO: 18)
HQAISPRTL;
(SEQ ID NO: 19)
NTMLNTVGGHQAAMQ;
(SEQ ID NO: 20)
VGGHQAAMQ;
(SEQ ID NO: 21)
STLQEQIGWMTNNPP;
(SEQ ID NO: 22)
STLQEQIGW;
(SEQ ID NO: 23)
IVRMYSPVSILDIRQ;
(SEQ ID NO: 24)
RMYSPVSIL;
(SEQ ID NO: 25)
QKQEPIDKELYPLAS;
(SEQ ID NO: 26)
KQEPIDKEL;
(SEQ ID NO: 27)
SFSFPQITLWQRPLV;
(SEQ ID NO: 28)
VRQYDQILIEICGKK;
(SEQ ID NO: 29)
EPFRKQNPDIVIYQL;
(SEQ ID NO: 30)
YVDGAANRETKLGKA;
(SEQ ID NO: 31)
EEHEKYSNWRAMAS;
or
(SEQ ID NO: 32)
ILDLWVYHTQGYFPD.

In some embodiments, the recombinant HCMV vector comprises a nucleic acid encoding one or more human immunodeficiency virus antigens. In some embodiments, the recombinant HCMV vector does not express UL128. In some embodiments, the recombinant HCMV vector does not express UL130. In some embodiments, the recombinant HCMV vector does not express UL128 and UL130. In some embodiments, the recombinant HCMV vector does not express UL146 and UL147. In some embodiments, the recombinant HCMV vector does not express UL18 protein, UL128 protein, UL130 protein, UL146 protein, and UL147 protein, or orthologs thereof, due to the presence of one or more mutations in the nucleic acid sequence encoding UL18, UL128, UL130, UL146, or UL147. In some embodiments, the mutations in the nucleic acid sequence encoding UL18, UL128, UL130, UL146, or UL147 are selected from the group consisting of point mutations, frameshift mutations, truncation mutations, and deletion of all of the nucleic acid sequence encoding the viral protein. In some embodiments, the recombinant HCMV vector further comprises a nucleic acid sequence encoding UL40, or an ortholog thereof. In some embodiments, the recombinant HCMV vector further comprises a nucleic acid sequence encoding US28, or an ortholog thereof. In some embodiments, the recombinant HCMV vector does not express UL82 (pp 71), or an ortholog thereof. In some embodiments, the recombinant HCMV vector does not express US11, or an ortholog thereof. In some embodiments, the recombinant HCMV vector further comprises a nucleic acid sequence encoding a microRNA (miRNA) recognition element (MRE), wherein the MRE contains a target site for a miRNA expressed in endothelial cells. In some embodiments, the miRNA expressed in endothelial cells is miR126, miR-126-3p, miR-130a, miR-210, miR-221/222, miR-378, miR-296, or miR-328. In some embodiments, the recombinant HCMV vector further comprises a nucleic acid sequence encoding a MRE, wherein the MRE contains a target site for a miRNA expressed in myeloid cells. In some embodiments, the miRNA expressed in myeloid cells is miR-142-3p, miR-223, miR-27a, miR-652, miR-155, miR-146a, miR-132, miR-21, or miR-125.

The CMV vectors disclosed herein may be used as an immunogenic or vaccine composition containing the recombinant CMV virus or vector, and a pharmaceutically acceptable carrier or diluent. An immunologic composition containing the recombinant CMV virus or vector (or an expression product thereof) elicits an immunological response—local or systemic. The response can, but need not be, protective. A vaccine composition elicits a local or systemic protective or therapeutic response. Accordingly, the term “immunogenic composition” includes a “vaccine composition” (as the former term may be a protective composition).

The recombinant CMV vectors disclosed herein may be used in methods of inducing an immunological response in a subject comprising administering to the subject an immunogenic, immunological or vaccine composition comprising the recombinant CMV virus or vector and a pharmaceutically acceptable carrier or diluent.

The recombinant CMV vectors disclosed herein may be used in therapeutic compositions containing the recombinant CMV virus or vector and a pharmaceutically acceptable carrier or diluent. The CMV vectors disclosed herein may be prepared by inserting DNA comprising a sequence that encodes the tumor antigen into an essential or non-essential region of the CMV genome. The method may further comprise deleting one or more regions from the CMV genome. The method may comprise in vivo recombination. Thus, the method may comprise transfecting a cell with CMV DNA in a cell-compatible medium in the presence of donor DNA comprising the heterologous DNA flanked by DNA sequences homologous with portions of the CMV genome, whereby the heterologous DNA is introduced into the genome of the CMV, and optionally then recovering CMV modified by the in vivo recombination. The method may also comprise cleaving CMV DNA to obtain cleaved CMV DNA, ligating the heterologous DNA to the cleaved CMV DNA to obtain hybrid CMV-heterologous DNA, transfecting a cell with the hybrid CMV-heterologous DNA, and optionally then recovering CMV modified by the presence of the heterologous DNA Since in vivo recombination is comprehended, the method accordingly also provides a plasmid comprising donor DNA not naturally occurring in CMV encoding a polypeptide foreign to CMV, the donor DNA is within a segment of CMV DNA that would otherwise be co-linear with an essential or non-essential region of the CMV genome such that DNA from an essential or nonessential region of CMV is flanking the donor DNA The heterologous DNA may be inserted into CMV to generate the recombinant CMV in any orientation that yields stable integration of that DNA, and expression thereof, when desired.

The DNA encoding the heterologous antigen in the recombinant CMV vector may also include a promoter. The promoter may be from any source such as a herpes virus, including an endogenous cytomegalovirus (CMV) promoter, such as a human CMV (HCMV), rhesus macaque CMV (RhCMV), murine, or other CMV promoter. The promoter may also be a nonviral promoter such as the EF1α promoter. The promoter may be a truncated transcriptionally active promoter which comprises a region transactivated with a transactivating protein provided by the virus and the minimal promoter region of the full-length promoter from which the truncated transcriptionally active promoter is derived. The promoter may be composed of an association of DNA sequences corresponding to the minimal promoter and upstream regulatory sequences. A minimal promoter is composed of the CAP site plus ATA box (minimum sequences for basic level of transcription; unregulated level of transcription); “upstream regulatory sequences” are composed of the upstream element(s) and enhancer sequence(s). Further, the term “truncated” indicates that the full-length promoter is not completely present, i.e., that some portion of the full-length promoter has been removed. And, the truncated promoter may be derived from a herpesvirus such as MCMV or HCMV, e.g., HCMV-IE or MCMV-IE. There may be up to a 40% and even up to a 90% reduction in size, from a full-length promoter, based upon base pairs. The promoter may also be a modified non-viral promoter. As to HCMV promoters, reference is made to U.S. Pat. Nos. 5,168,062 and 5,385,839. As to transfecting cells with plasmid DNA for expression therefrom, reference is made to Feigner et al. (1994), J Biol. Chem. 269, 2550-2561. And, as to direct injection of plasmid DNA as a simple and effective method of vaccination against a variety of infectious diseases reference is made to Science, 259:1745-49, 1993. It is therefore within the scope of this disclosure that the vector may be used by the direct injection of vector DNA.

Also disclosed is an expression cassette that may be inserted into a recombinant virus or plasmid comprising the truncated transcriptionally active promoter. The expression cassette may further include a functional truncated polyadenylation signal; for instance an SV40 polyadenylation signal which is truncated, yet functional. Considering that nature provided a larger signal, it is indeed surprising that a truncated polyadenylation signal is functional. A truncated polyadenylation signal addresses the insert size limit problems of recombinant viruses such as CMV. The expression cassette may also include heterologous DNA with respect to the virus or system into which it is inserted; and that DNA may be heterologous DNA as described herein.

As to antigens for use in vaccine or immunological compositions, see also Stedman's Medical Dictionary (24th edition, 1982, e.g., definition of vaccine (for a list of antigens used in vaccine formulations); such antigens or epitopes of interest from those antigens may be used. As to tumor antigens, one skilled in the art may select a tumor antigen and the coding DNA therefor from the knowledge of the amino acid and corresponding DNA sequences of the peptide or polypeptide, as well as from the nature of particular amino acids (e.g., size, charge, etc.) and the codon dictionary, without undue experimentation.

One method to determine T epitopes of an antigen involves epitope mapping. Overlapping peptides of the tumor antigen are generated by oligo-peptide synthesis. The individual peptides are then tested for their ability to induce T cell activation. This approach has been particularly useful in mapping T cell epitopes since the T cell recognizes short linear peptides complexed with MEW molecules.

An immune response to a tumor antigen is generated, in general, as follows: T cells recognize proteins only when the protein has been cleaved into smaller peptides and is presented in a complex called the “major histocompatibility complex (MHC)” located on another cell's surface. There are two classes of MHC complexes—class I and class II, and each class is made up of many different alleles. Different species, and individual subjects have different types of MHC complex alleles; they are said to have a different MEW type. One type of MEW class I molecule is called MHC-E (HLA-E in humans, Mamu-E in RM, Qa-lb in mice). Unlike other MHC-I molecules, MHC-E is highly conserved within and between mammalian species.

It is noted that the DNA comprising the sequence encoding the tumor antigen may itself include a promoter for driving expression in the CMV vector or the DNA may be limited to the coding DNA of the tumor antigen. This construct may be placed in such an orientation relative to an endogenous CMV promoter that it is operably linked to the promoter and is thereby expressed. Further, multiple copies of DNA encoding the tumor antigen or use of a strong or early promoter or early and late promoter, or any combination thereof, may be done so as to amplify or increase expression. Thus, the DNA encoding the tumor antigen may be suitably positioned with respect to a CMV endogenous promoter, or those promoters may be translocated to be inserted at another location together with the DNA encoding the tumor antigen. Nucleic acids encoding more than one tumor antigen may be packaged in the CMV vector.

Further disclosed are pharmaceutical and other compositions containing the disclosed CMV vectors. Such pharmaceutical and other compositions may be formulated so as to be used in any administration procedure known in the art. Such pharmaceutical compositions may be via a parenteral route (intradermal, intraperitoneal, intramuscular, subcutaneous, intravenous, or others). The administration may also be via a mucosal route, e.g., oral, nasal, genital, etc.

The disclosed pharmaceutical compositions may be prepared in accordance with standard techniques well known to those skilled in the pharmaceutical arts. Such compositions may be administered in dosages and by techniques well known to those skilled in the medical arts taking into consideration such factors as the breed or species, age, sex, weight, and condition of the particular patient, and the route of administration. The compositions may be administered alone, or may be co-administered or sequentially administered with other CMV vectors or with other immunological, antigenic or vaccine or therapeutic compositions. Such other compositions may include purified native antigens or epitopes or antigens or epitopes from the expression by a recombinant CMV or another vector system; and are administered taking into account the aforementioned factors.

Examples of compositions include liquid preparations for orifice, e.g., oral, nasal, anal, genital, e.g., vaginal, etc., administration such as suspensions, syrups or elixirs; and, preparations for parenteral, subcutaneous, intraperitoneal, intradermal, intramuscular or intravenous administration (e.g., injectable administration) such as sterile suspensions or emulsions. In such compositions the recombinant may be in admixture with a suitable carrier, diluent, or excipient such as sterile water, physiological saline, glucose or the like.

Antigenic, immunological or vaccine compositions typically may contain an adjuvant and an amount of the CMV vector or expression product to elicit the desired response. In human applications, alum (aluminum phosphate or aluminum hydroxide) is a typical adjuvant. Saponin and its purified component Quil A, Freund's complete adjuvant and other adjuvants used in research and veterinary applications have toxicities which limit their potential use in human vaccines. Chemically defined preparations such as muramyl dipeptide, monophosphoryllipid A, phospholipid conjugates such as those described by Goodman-Snitkoff et al., J Immunol. 147:410-415 (1991), encapsulation of the protein within a proteoliposome as described by Miller et al., J Exp. Med. 176:1739-1744 (1992), and encapsulation of the protein in lipid vesicles such as Novasome lipid vesicles (Micro Vescular Systems, Inc., Nashua, N.H.) may also be used.

The composition may be packaged in a single dosage form for immunization by parenteral (e.g., intramuscular, intradermal or subcutaneous) administration or orifice administration, e.g., perlingual (e.g., oral), intragastric, mucosal including intraoral, intraanal, intravaginal, and the like administration. And again, the effective dosage and route of administration are determined by the nature of the composition, by the nature of the expression product, by expression level if recombinant CMV is directly used, and by known factors, such as breed or species, age, sex, weight, condition and nature of host, as well as LD50 and other screening procedures which are known and do not require undue experimentation. Dosages of expressed product may range from a few to a few hundred micrograms, e.g., 5 to 500 μg. The CMV vector may be administered in any suitable amount to achieve expression at these dosage levels. In nonlimiting examples: CMV vectors may be administered in an amount of at least 10₂ pfu; thus, CMV vectors may be administered in at least this amount; or in a range from about 10₂ pfu to about 10₇ pfu. Other suitable carriers or diluents may be water or a buffered saline, with or without a preservative. The CMV vector may be lyophilized for resuspension at the time of administration or may be in solution. “About” may mean within 1%, 5%, 10% or 20% of a defined value.

It should be understood that the proteins and the nucleic acids encoding them of the present disclosure may differ from the exact sequences illustrated and described herein. Thus, the disclosure contemplates deletions, additions, truncations, and substitutions to the sequences shown, so long as the sequences function in accordance with the methods of the disclosure. In this regard, substitutions will generally be conservative in nature, i.e., those substitutions that take place within a family of amino acids. For example, amino acids are generally divided into four families: (1) acidic—aspartate and glutamate; (2) basic—lysine, arginine, and histidine; (3) nonpolar—alanine, valine, leucine, isoleucine, proline, phenylalanine, methionine, and tryptophan; and (4) uncharged polar—glycine, asparagine, glutamine, cysteine, serine threonine, and tyrosine. Phenylalanine, tryptophan, and tyrosine are sometimes classified as aromatic amino acids. It is reasonably predictable that an isolated replacement of leucine with isoleucine or valine, or vice versa; an aspartate with a glutamate or vice versa; a threonine with a serine or vice versa; or a similar conservative replacement of an amino acid with a structurally related amino acid, will not have a major effect on the biological activity. Proteins having substantially the same amino acid sequence as the proteins described but possessing minor amino acid substitutions that do not substantially affect the immunogenicity of the protein are, therefore, within the scope of the disclosure.

The nucleotide sequences of the present disclosure may be codon optimized, for example the codons may be optimized for use in human cells. For example, any viral or bacterial sequence may be so altered. Many viruses, including HIV and other lentiviruses, use a large number of rare codons and, by altering these codons to correspond to codons commonly used in the desired subject, enhanced expression of the tumor antigen may be achieved as described in Andre et al., J Virol. 72:1497-1503, 1998.

Nucleotide sequences encoding functionally and/or antigenically equivalent variants and derivatives of the CMV vectors and the glycoproteins included therein are contemplated. These functionally equivalent variants, derivatives, and fragments display the ability to retain antigenic activity. For instance, changes in a DNA sequence that do not change the encoded amino acid sequence, as well as those that result in conservative substitutions of amino acid residues, one or a few amino acid deletions or additions, and substitution of amino acid residues by amino acid analogs are those which will not significantly affect properties of the encoded polypeptide. Conservative amino acid substitutions are glycine/alanine; valine/isoleucine/leucine; asparagine/glutamine; aspartic acid/glutamic acid; serine/threonine/methionine; lysine/arginine; and phenylalanine/tyrosine/tryptophan. In some embodiments, the variants have at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% homology or identity to the antigen, epitope, immunogen, peptide or polypeptide of interest.

Sequence identity or homology is determined by comparing the sequences when aligned so as to maximize overlap and identity while minimizing sequence gaps. In particular, sequence identity may be determined using any of a number of mathematical algorithms. A nonlimiting example of a mathematical algorithm used for comparison of two sequences is the algorithm of Karlin & Altschul, Proc. Natl. Acad. Sci. USA 1990; 87: 2264-2268, modified as in Karlin & Altschul, Proc. Natl. Acad. Sci. USA 1993; 90: 5873-5877.

Another example of a mathematical algorithm used for comparison of sequences is the algorithm of Myers & Miller, CABIOS 1988; 4: 11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 may be used. Yet another useful algorithm for identifying regions of local sequence similarity and alignment is the FASTA algorithm as described in Pearson & Lipman, Proc. Natl. Acad. Sci. USA 1988; 85: 2444-2448.

Advantageous for use according to the present disclosure is the WU—BLAST (Washington University BLAST) version 2.0 software. WU-BLAST version 2.0 executable programs for several UNIX platforms may be downloaded. This program is based on WU-BLAST version 1.4, which in turn is based on the public domain NCBI-BLAST version 1.4 (Altschul & Gish, 1996, Local alignment statistics, Doolittle ed., Methods in Enzymology 266: 460-480; Altschul et al., Journal of Molecular Biology 1990; 215: 403-410; Gish & States, 1993; Nature Genetics 3: 266-272; Karlin & Altschul, 1993; Proc. Natl. Acad. Sci. USA 90: 5873-5877; all of which are incorporated by reference herein).

The various recombinant nucleotide sequences and antibodies and/or antigens of the disclosure are made using standard recombinant DNA and cloning techniques. Such techniques are well known to those of skill in the art. See for example, “Molecular Cloning: A Laboratory Manual”, second edition (Sambrook et al. 1989).

Any vector that allows expression of the viruses of the present disclosure may be used in accordance with the present disclosure. In certain embodiments, the disclosed viruses may be used in vitro (such as using cell-free expression systems) and/or in cultured cells grown in vitro in order to produce the encoded heterologous antigen (e.g., tumor virus antigens, HIV antigens, tumor antigens, and antibodies) which may then be used for various applications such as in the production of proteinaceous vaccines. For such applications, any vector that allows expression of the virus in vitro and/or in cultured cells may be used.

For the disclosed tumor antigens to be expressed, the protein coding sequence of the tumor antigen should be “operably linked” to regulatory or nucleic acid control sequences that direct transcription and translation of the protein. As used herein, a coding sequence and a nucleic acid control sequence or promoter are said to be “operably linked” when they are covalently linked in such a way as to place the expression or transcription and/or translation of the coding sequence under the influence or control of the nucleic acid control sequence. The “nucleic acid control sequence” may be any nucleic acid element, such as, but not limited to promoters, enhancers, IRES, introns, and other elements described herein that direct the expression of a nucleic acid sequence or coding sequence that is operably linked thereto. The term “promoter” will be used herein to refer to a group of transcriptional control modules that are clustered around the initiation site for RNA polymerase II and that when operationally linked to the protein coding sequences of the disclosure lead to the expression of the encoded protein. The expression of the transgenes of the present disclosure may be under the control of a constitutive promoter or of an inducible promoter, which initiates transcription only when exposed to some particular external stimulus, such as, without limitation, antibiotics such as tetracycline, hormones such as ecdysone, or heavy metals. The promoter may also be specific to a particular cell-type, tissue or organ. Many suitable promoters and enhancers are known in the art, and any such suitable promoter or enhancer may be used for expression of the transgenes of the disclosure. For example, suitable promoters and/or enhancers may be selected from the Eukaryotic Promoter Database (EPDB).

The vectors used in accordance with the present disclosure may contain a suitable gene regulatory region, such as a promoter or enhancer, such that the antigens of the disclosure may be expressed.

The CMV vectors described herein may contain mutations that may prevent host to host spread, thereby rendering the virus unable to infect immunocompromised or other subjects that could face complications as a result of CMV infection. The CMV vectors described herein may also contain mutations that result in the presentation of immunodominant and nonimmunodominant epitopes as well as non-canonical MHC restriction. However, mutations in the CMV vectors described herein do not affect the ability of the vector to reinfect a subject that has been previously infected with CMV. Such CMV mutations are described in, for example, US Patent Publications 2013-013676S; 2010-0142S23; 2014-014103S; and PCT application publication WO 2014/13S209, all of which are incorporated by reference herein.

The disclosed CMV vectors may be administered in vivo, for example where the aim is to produce an immunogenic response, including a CD8+ immune response, including an immune response characterized by a high percentage of the CD8+ T cell response being restricted by MHC-E, MHC-II, or MHC-I (or a homolog or ortholog thereof). For example, in some examples it may be desired to use the disclosed CMV vectors in a laboratory animal, such as rhesus macaques for preclinical testing of immunogenic compositions and vaccines using RhCMV. In other examples, it will be desirable to use the disclosed CMV vectors in human subjects, such as in clinical trials and for actual clinical use of the immunogenic compositions using HCMV.

For such in vivo applications the disclosed CMV vectors are administered as a component of an immunogenic composition further comprising a pharmaceutically acceptable carrier. In some embodiments, the immunogenic compositions of the disclosure are useful to stimulate an immune response against the heterologous antigen, including a tumor antigen, a tumor virus antigen, or a host self-antigen and may be used as one or more components of a prophylactic or therapeutic vaccine against tumor antigens, tumor virus antigens, or host self antigens for the prevention, amelioration or treatment of cancer. The nucleic acids and vectors of the disclosure are particularly useful for providing genetic vaccines, i.e., vaccines for delivering the nucleic acids encoding the antigens of the disclosure to a subject, such as a human, such that the antigens are then expressed in the subject to elicit an immune response.

Immunization schedules (or regimens) are well known for animals (including humans) and may be readily determined for the particular subject and immunogenic composition. Hence, the immunogens may be administered one or more times to the subject. Preferably, there is a set time interval between separate administrations of the immunogenic composition. While this interval varies for every subject, typically it ranges from 10 days to several weeks, and is often 2, 4, 6, or 8 weeks. For humans, the interval is typically from 2 to 6 weeks. In a particularly advantageous embodiment of the present disclosure, the interval is longer, advantageously about 10 weeks, 12 weeks, 14 weeks, 16 weeks, 18 weeks, 20 weeks, 22 weeks, 24 weeks, 26 weeks, 28 weeks, 30 weeks, 32 weeks, 34 weeks, 36 weeks, 38 weeks, 40 weeks, 42 weeks, 44 weeks, 46 weeks, 48 weeks, 50 weeks, 52 weeks, 54 weeks, 56 weeks, 58 weeks, 60 weeks, 62 weeks, 64 weeks, 66 weeks, 68 weeks, or 70 weeks. The immunization regimes typically have from 1 to 6 administrations of the immunogenic composition, but may have as few as one or two or four. The methods of inducing an immune response may also include administration of an adjuvant with the immunogens. In some instances, annual, biannual or other long interval (5-10 years) booster immunization may supplement the initial immunization protocol. The present methods also include a variety of prime-boost regimens. In these methods, one or more priming immunizations are followed by one or more boosting immunizations. The actual immunogenic composition may be the same or different for each immunization and the type of immunogenic composition (e.g., containing protein or expression vector), the route, and formulation of the immunogens may also be varied. For example, if an expression vector is used for the priming and boosting steps, it may either be of the same or different type (e.g., DNA or bacterial or viral expression vector). One useful prime-boost regimen provides for two priming immunizations, four weeks apart, followed by two boosting immunizations at 4 and 8 weeks after the last priming immunization. It should also be readily apparent to one of skill in the art that there are several permutations and combinations that are encompassed using the DNA, bacterial and viral expression vectors of the disclosure to provide priming and boosting regimens. CMV vectors may be used repeatedly while expressing different antigens derived from different pathogens.

### EXAMPLES

### Example 1: Protection Against SIV by Induction of MHC-E Restricted CD8+ T Cells

In several studies it was demonstrated that strain 68-1 derived RhCMV vectors expressing SIV antigens control and ultimately eliminate infection by highly pathogenic SIVmac239 (Hansen 2019. A live-attenuated RhCMV/SIV vaccine shows long-term efficacy against heterologous SIV challenge. Science Translational Medicine 11:eaaw2607; Hansen 2013. Immune clearance of highly pathogenic SIV infection. Nature 502:100-4). This protection correlated with the ability of strain 68-1 RhCMV to elicit MHC-II and MHC-E restricted CD8+ T cells Hansen 2016. Broadly targeted CD8(+) T cell responses restricted by major histocompatibility complex E. Science 351:714-20; Hansen. Cytomegalovirus Vectors Violate CD8+ T Cell Epitope Recognition Paradigms. Science 340:1237874-1237874) However, it was not known whether MHC-II and/or MHC-E restricted CD8+ T cells are necessary for this protection.

Therefore, the ability to specifically program CD8+ T cells that are restricted exclusively by MHC-E or MHC-II enabled the examination of whether MHC-E or MHC-II restricted CD8+ T cells are responsible for the unique protection against SIVmac239. Four rhesus macaque (RM) cohorts were inoculated with different 68-1 RhCMV strains as described below.

Cohort 1: Nine RM were inoculated with three 68-1 RhCMV “MHC-E only” vectors each carrying three recognition sites for mir126 in the 3′ untranslated region of the essential genes Rh108 (UL79) and Rh156 (IE2) and expressing (one insert per vector) the SIV antigens SIVgag, SIVretanef (fusion of rev, tat, and nef), and the 5′ segment of SIVpol, respectively.

Cohort 2: 15 RM were inoculated with three 68-1 RhCMV “MHC-II only” vectors deleted for Rh67 (UL40) and expressing (one insert per vector) the SIV antigens SIVgag, SIVretanef (fusion of rev, tat, and nef) and the 5′ segment of SIVpol, respectively.

Cohort 3: 12 RM were inoculated with three 68-1 RhCMV “MHC-II only” vectors each carrying three recognition sites for mir142 in the 3′ untranslated region of the essential genes Rh108 (UL79) and Rh156 (IE2) and expressing (one insert per vector) the SIV antigens SIVgag, SIVretanef (fusion of rev, tat, and nef) and the 5′ segment of SIVpol, respectively.

Cohort 4: (control cohort) 15 RM were inoculated with three 68-1 RhCMV vectors expressing (one insert per vector) the SIV antigens SIVgag, SIVretanef (fusion of rev, tat, and nef) and the 5′ segment of SIVpol, respectively.

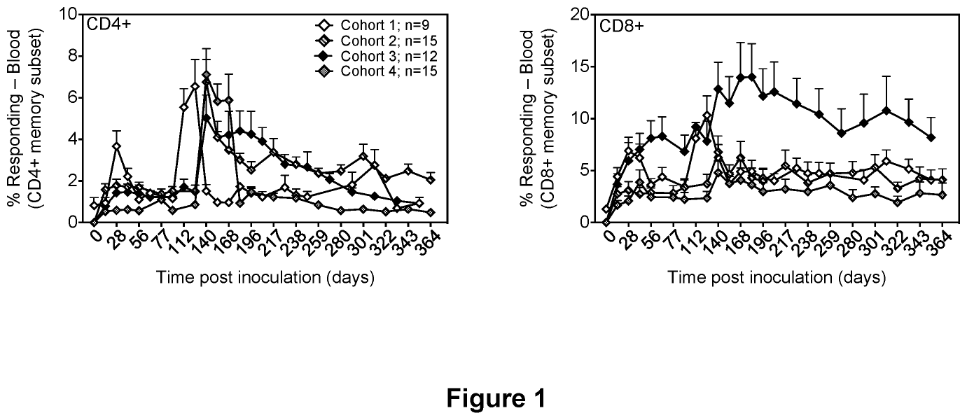

Average frequencies of CD4+ or CD8+ T cells responding to SIV-antigen derived peptide pools were quantified. T cell frequencies were determined in peripheral blood mononuclear cells (PBMC) at the indicated time points by intracellular cytokine staining for IFNγ or TNFα in the presence of pools of overlapping (by 11A) 15mer peptides representing the SIV antigens. Each of the RM developed robust CD4+ and CD8+ T cell responses to each of the SIV antigens (FIG. 1).

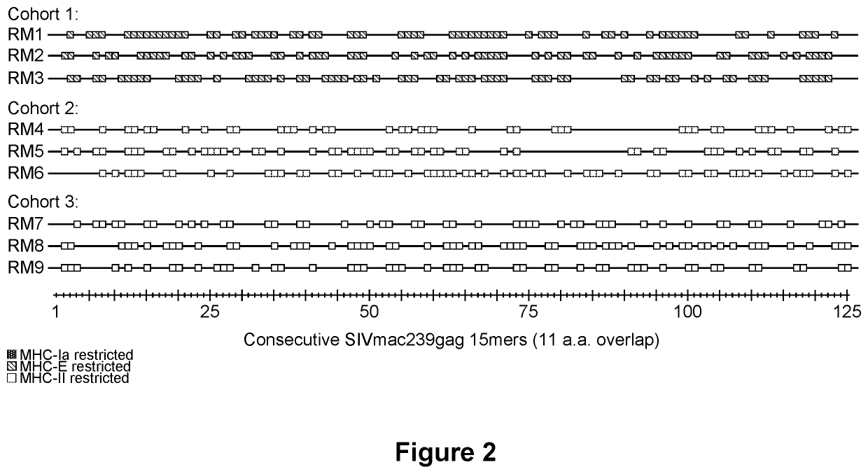

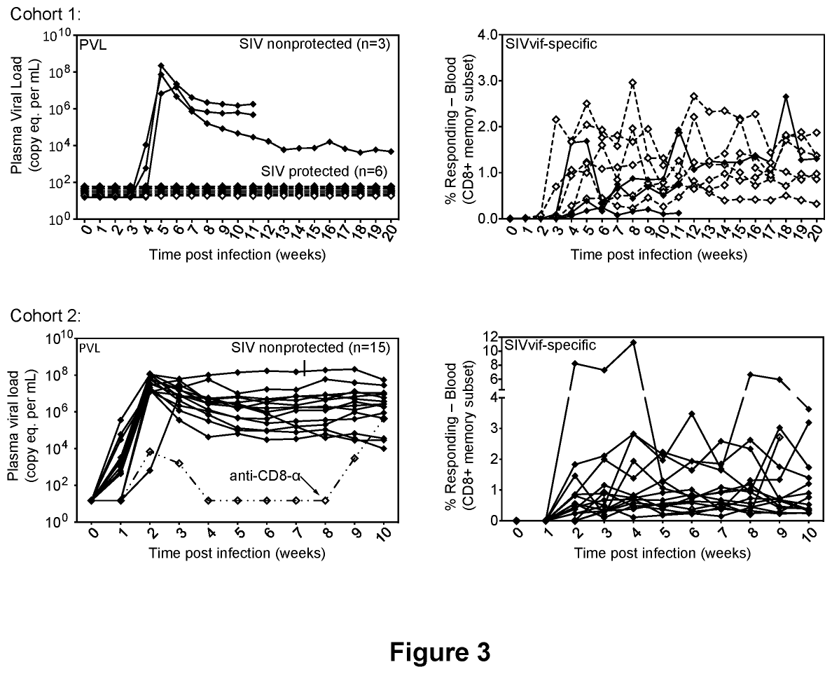

Next, the MHC-restriction of the SIVgag-specific CD8+ T cell responses was analyzed. SIVgag-specific CD8+ T cell responses in PBMC obtained from three RM in each of the indicated cohorts were measured in the presence of individual peptides. MHC restriction was determined by blocking with the anti-pan-MHC-I mAb W6/32, the MHC-E blocking peptide VL9, and the MHC-II blocking peptide CLIP. Whereas all peptide responses in cohort 1 animals were blocked by VL9 peptide, peptide responses in cohorts 2 and 3 were blocked by CLIP peptide (FIG. 2). Thus, CD8+ T cells in cohort 1 are exclusively restricted by MHC-E whereas CD8+ T cells in cohorts 2 and 3 are exclusively restricted by MHC-II. CD8+ T cell responses in cohort 4 animals (not shown) are restricted by both MHC-II and MHC-E as previously reported (Hansen 2016. Broadly targeted CD8(+) T cell responses restricted by major histocompatibility complex E. Science 351:714-20; Hansen 2013. Cytomegalovirus Vectors Violate CD8+ T Cell Epitope Recognition Paradigms. Science 340:1237874-1237874).

To determine whether MHC-E or MHC-II-restricted CD8+ T cells were responsible for protection, cohorts 1, 2, and 3 were challenged by repeated, limiting dose intra-rectal inoculation of SIVmac239. RM were challenged weekly until the first plasma viral load (pvl) or SIVvif responses were detected (with the start of infection designated as the previous challenge). Since the vaccine vectors do not express SIVvif, the development of de novo SIVvif responses are proof for infection in the absence of detectable SIV plasma viral load. RM were considered controllers (white boxes) if plasma viremia was never observed or became undetectable within 2 weeks of the initial positive pvl and was then maintained below threshold for at least 4 of the subsequent 5 weeks, in contrast to non-controllers (black boxes), which once infected, manifested continuous viremia with a typical peak and plateau pattern.

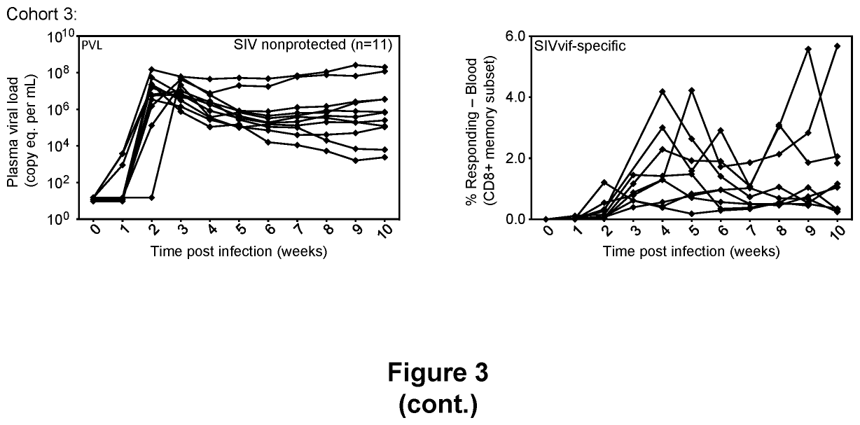

All animals in cohorts 2 and 3 developed systemic, progressive SIV viremia suggesting that MHC-II restricted CD8+ T cells were unable to provide protection against SIVmac239 infection (FIG. 3). In contrast, 6/9 (67%) of cohort 1 animals vaccinated with 68-1 RhCMV/SIV/miR126 vectors stringently controlled infection with SIVmac239. These data demonstrate that MHC-E restricted CD8+ T cell responses provided protection against highly virulent SIV.

It was previously demonstrated that strain 68-1 derived RhCMV vectors elicit CD8+ T cell responses that display an unusually high epitope density (=number of peptides recognized by T cells within a given antigen) (Hansen. 2013. Cytomegalovirus Vectors Violate CD8+ T Cell Epitope Recognition Paradigms. Science 340:1237874-1237874). It was further shown that some of these MHC-E and MHC-II epitopes, so called supertopes, are recognized in every animal (Hansen 2016. Broadly targeted CD8(+) T cell responses restricted by major histocompatibility complex E. Science 351:714-20). Supertopes have not been described for “classical” epitopes, presented by MHC-I molecules, and thus represent a unique feature of CMV-based vectors. To determine whether supertopes alone could account for the protection observed with “MHC-E only” RhCMV vectors described above, an artificial fusion protein was generated consisting of supertope sequences from individual SIV antigens (Table 1, 15mer and minimal supertope peptide sequences are underlined).

TABLE 1
MHC-E supertopes in each SIV antigen

| | MHC | | SEQ ID |
|---|---|---|---|
| Antigen | Restriction | Peptide Sequence | NO: |
| SIVrev | MHC-E | RRWRRRWQQLLALADRIYSFPDP | 1 |
| SIVtat | MHC-E | TSSASNKPISNRTRHCQPE | 2 |
| SIVnef | MHC-E | ISMRRSRPSGDLRQRLLRA | 3 |
| SIVnef | MHC-E | EKLAYRKQNMDDIDEEDDD | 4 |
| SIVnef | MHC-E | AQTSQWDDPWGEVLAWKFD | 5 |
| SIVnef | MEW-E | YVRYPEEFGSKSGLSEEEV | 6 |
| SIVpol | MHC-E | GGIGGFINTKEYKNVEIEVLGKR | 7 |
| SIVpol | MEW-E | NTPTFAIKKKDKNKWRMLIDFRE | 8 |
| SIVpol | MEW-E | WMGYELWPTKWKLQKIELP | 9 |
| SIVgag | MEW-E | LGLQKCVRMYNPTNILDVK | 10 |
| SIVgag | MEW-E | YMQLGKQQREKQRESREKPYKEV | 11 |

The sequence of the artificial fusion protein is as follows (HA-epitope tag is underlined):

(SEQ ID NO: 12)
MRRWRRRWQQLLALADRIYSFPDPTSSASNKPISNRTRHCQPEISMRRSR
PSGDLRQRLLRAEKLAYRKQNMDDIDEEDDDAQTSQWDDPWGEVLAWKFD
YVRYPEEFGSKSGLSEEEVGGIGGFINTKEYKNVEIVLGKRNTPTFAIKK
KDKNKWRMLIDFREWMGYELWPTKWKLQKIELPLGLQKCVRMYNPTNILD
VKYMQLGKQQREKQRESREKPYKEVYPYDVPDYAD.

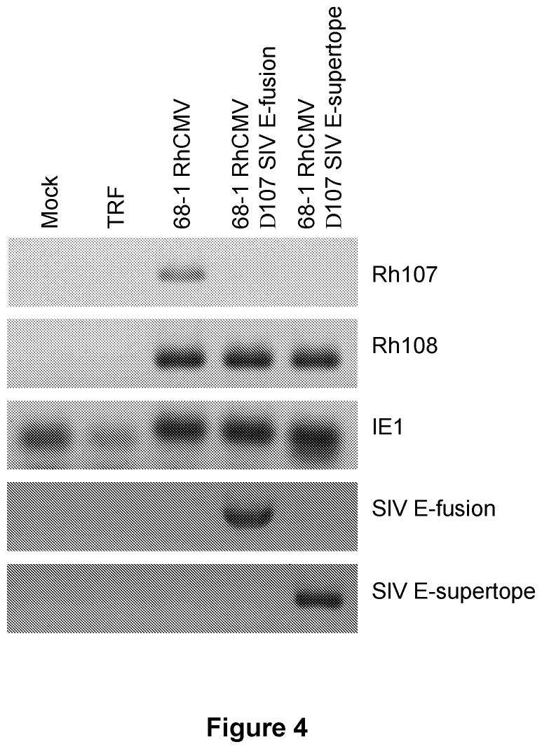

Immunoblotting was performed to demonstrate the expression of the SIV supertope fusion construct by probing with an anti-HA antibody (FIG. 4).

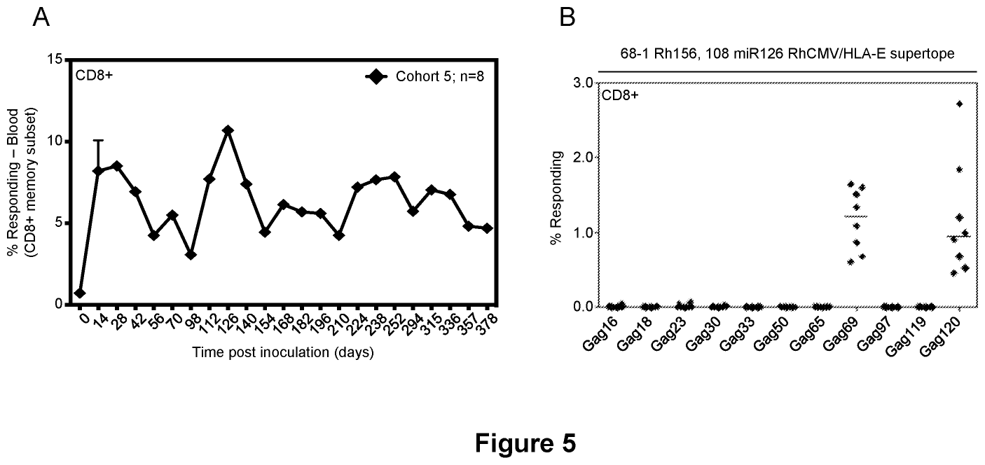

The SIV MHC-E supertope fusion protein was inserted into 68-1 RhCMV containing mir126 targeting sites with the goal to focus the CD8+ T cell responses onto a small set of MHC-E restricted epitopes. The resulting construct was inoculated into 8 RM (Cohort 5). T cell frequencies were determined in peripheral blood mononuclear cells (PBMC) at the indicated time points by intracellular cytokine staining for IFNγ or TNFα in the presence of pools of individual 15mer peptides representing the SIV supertopes (FIG. 5). CD8+ T cells were responsive to SIV-antigen derived peptides (FIG. 5A). The CD8+ T cells were responsive to the MHC-E-restricted supertopes Gag69 and Gag120 but not other MHC-E-restricted Gag epitopes that are commonly recognized by CD8+ T cells from RM immunized with 68-1 RhCMV/gag vectors expressing whole SIVgag inserts (FIG. 5B). These results show that all animals elicited SIV-specific CD8+ T cell responses that were exclusively directed to supertopes.

To determine whether MHC-E supertope-restricted CD8+ T cells would be able to replicate the protection observed with “MHC-E-only” vectors, cohort 5 was challenged by repeated low dose intra-rectal inoculation of SIVmac239 as described above. RM were challenged weekly until the first plasma viral load (pvl) or SIVvif responses were detected (with the start of infection designated as the previous challenge). RM were considered controllers (boxes) if pvl became undetectable within 2 weeks of the initial positive pvl and was then maintained below threshold for at least 4 of the subsequent 5 weeks, in contrast to non-controllers (black boxes), which once infected, manifested continuous viremia with a typical peak and plateau pattern.

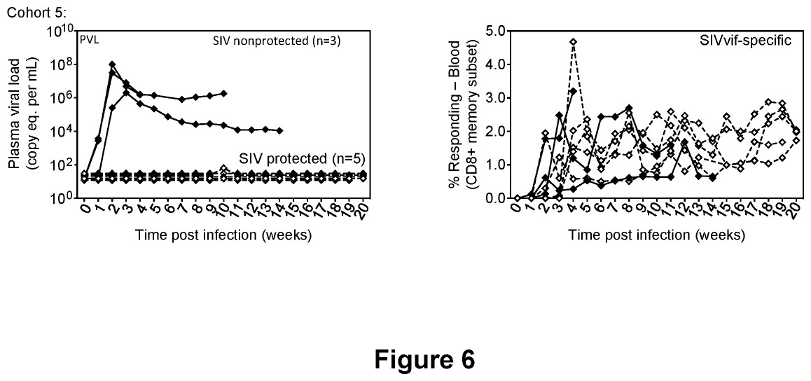

Importantly, 5/7 (71%) of animals vaccinated with a single 68-1 RhCMV/SIV/miR126 vector expressing the supertope-fusion protein controlled infection with SIVmac239 (FIG. 6). These data indicate that CD8+ T cells specific for MHC-E supertopes are responsible for protection against highly pathogenic SIV.

In order to design HIV-based supertope antigens, HIV supertopes were mapped by inserting HIV antigens into 68-1 RhCMV and inoculating RM. Table 2 contains a list of HIV supertopes identified. The optimal minimal peptide sequence is underlined.

TABLE 2
List of HIV supertopes.

| | | | | SEQ ID |
|---|---|---|---|---|
| | | | Peptide | NOs: |
| | | | Sequence | (full |
| | | | (15mer) | peptide |
| | | | (optimal | sequence, |
| | | | minimal | optimal |
| | | MHC- | peptide | minimal |
| | | Restric- | sequence is | peptide |
| Antigen | Peptide | tion | underlined) | sequence) |
| HIVgag | 4 | E | LDAWEKIRLRPGGKK | 13, 14 |
| | 29 | E | KKAQQAAADTGNSSQ | 15, 16 |
| | 36 | E | QMVHQAISPRTLNAW | 17, 18 |
| | 47 | E | NTMLNTVGGHQAAMQ | 19, 20 |
| | 61 | E | STLQEQIGWMTNNPP | 21, 22 |
| | 69 | E | IVRMYSPVSILDIRQ | 23, 24 |
| | 119 | E | QKQEPIDKELYPLAS | 25, 26 |
| HIVpol | 14 | E | SFSFPQITLWQRPLV | 27 |
| | 28 | E | VRQYDQILIEICGKK | 28 |
| | 81 | E | EPFRKQNPDIVIYQL | 29 |
| | 148 | E | YVDGAANRETKLGKA | 30 |
| | 180 | E | EEHEKYSNWRAMAS | 31 |
| HIVnef | 28 | E | ILDLWVYHTQGYFPD | 32 |

### Example 2: Expression of UL18 Prevents the Induction of MHC-E and MHC-II Restricted Cd8+ T Cells

To determine the impact of UL18 on the ability of strain 68-1 RhCMV vectors to elicit MHC-II and MHC-E restricted CD8+ T cell responses two RhCMV constructs were generated:

Construct 1: 68-1 RhCMV containing an expression cassette for the 5′ fragment of SIVpol under control of the EF1α promoter in RhCMV gene Rh211 as a vector backbone. UL18 was inserted by replacing the gene Rh13.1, thus UL18 would be expressed instead of Rh13.1. The UL18 sequence inserted corresponds to UL18 of the HCMV TR isolate.

Construct 2: 68-1 RhCMV in which the gene Rh107 (homolog of HCMV UL78) was replaced with a fusion protein of SIV rev, tat, and nef (SIVrtn) as a vector backbone. UL18 was inserted by replacing the gene Rh13.1.

5×10⁶ plaque forming units (PFU) of construct 1 were inoculated into three RhCMV-seropositive RM and the same amount of construct 2 was inoculated into two RhCMV-seropositive RM on day 0. For control, RM were inoculated with 68-1 RhCMV expressing SIVgag under control of the EF1α promoter.

On day 7, day 14, and biweekly after that, PBMC were isolated from two RM and the CD8+ T cell responses to the SIV antigens elicited by construct 1, 2, or control were measured by intracellular cytokine staining (ICS) for IFNγ and TNFα using overlapping 15mer peptide pools that covered SIVpol, SIVrtn or SIVgag, respectively. To specifically detect CD8+ T cells that recognized peptides in the context of MHC-E or MHC-II it was advantageous that supertopes within each SIV antigen are shared by all animals (Hansen Science 2013, Hansen Science 2016). Thus, each of the supertope peptides was tested individually by ICS in PBMC of the respective RM.

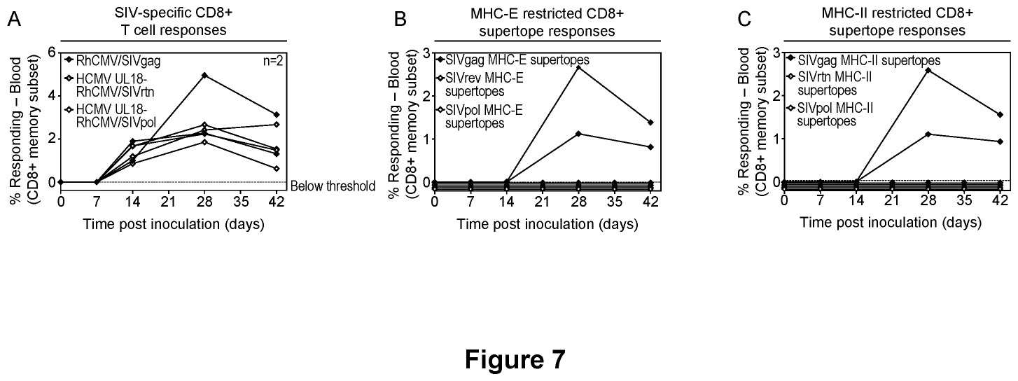

Frequencies of CD8+ T cells responding to SIV antigen peptide pools thus representing total antigen-specific responses in two animals from each group were analyzed (FIG. 7A). Frequencies of CD8+ T cells responding to MHC-E restricted supertopes and MHC-II restricted supertopes were also analyzed for the same two animals (FIGS. 7B, 7C).

All animals developed CD8+ T cell responses to the SIV antigen expressed by the RhCMV vector used for inoculation. However, supertope responses were only observed for 68-1 RhCMV/SIVgag whereas both vectors expressing UL18 did not elicit T cells recognizing supertopes. These results thus indicated that UL18 prevented the induction of MHC-E and MHC-II restricted CD8+ T cells.

Next, MHC-restriction mapping was performed to further determine which MHC molecules were responsible for the elicitation of SIVpol-specific responses in the three animals that received UL18 expressing 68-1 RhCMV/SIVpol. SIVpol-specific CD8+ T cell responses in PBMC obtained from three RM inoculated with construct 1 were measured in the presence of individual peptides. The CD8+ T cell responses to individual peptides within SIVpol were measured in the presence of specific reagents that either block MHC-I, MHC-II, or MHC-E presentation (MHC-I and MHC-E is blocked with antibody W6/32, MHC-II is blocked with HLA-DR-specific antibody and CLIP peptide, MHC-E is blocked with VL9 peptide).

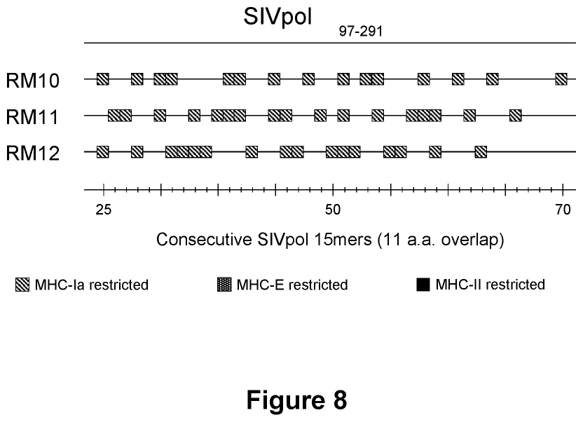

The results shown in FIG. 8 reveal that the stimulation of CD8+ T cells by each individual peptide was inhibited by pan-MHC-I inhibitory antibody W6/32, but not by MHC-E specific peptide VL9 or MHC-II specific antibodies and CLIP peptide. Thus, all CD8+ T cell epitopes are restricted by MHC-I. In contrast, CD8+ T cells from animals inoculated with 68-1 RhCMV expressing SIV antigens recognize all peptides in the context of MHC-II or MHC-E (Hansen Science 2013, Hansen Science 2016).

These results show that UL18 reprogrammed the CD8+ T cell response most likely by preventing the induction of MHC-II and MHC-E restricted CD8+ T cells. UL18 is known to engage the host inhibitory receptor LIR-1 (Yang Z, Bjorkman P J. 2008. Structure of UL18, a peptide-binding viral MHC mimic, bound to a host inhibitory receptor. Proc Natl Acad Sci USA 105:10095-100; Chapman T L, Heikeman A P, Bjorkman P J. 1999. The inhibitory receptor LIR-1 uses a common binding interaction to recognize class I MHC molecules and the viral homolog UL18. Immunity 11:603-13). A possible mechanism for this reprogramming is, therefore, that by engaging inhibitory Leukocyte inhibitory receptors (LIRs) on T cells, UL18 prevents the direct priming of CD8+ T cells by 68-1 RhCMV (direct priming refers to T cells being primed by infected cells). In the absence of direct priming, CD8+ T cells are elicited by cross-priming, i.e., indirectly by non-infected cells (e.g., dendritic cells) presenting antigen obtained from infected cells. Up to now, UL18 has not been implicated in preventing T cell priming. These results are thus unexpected and unprecedented.

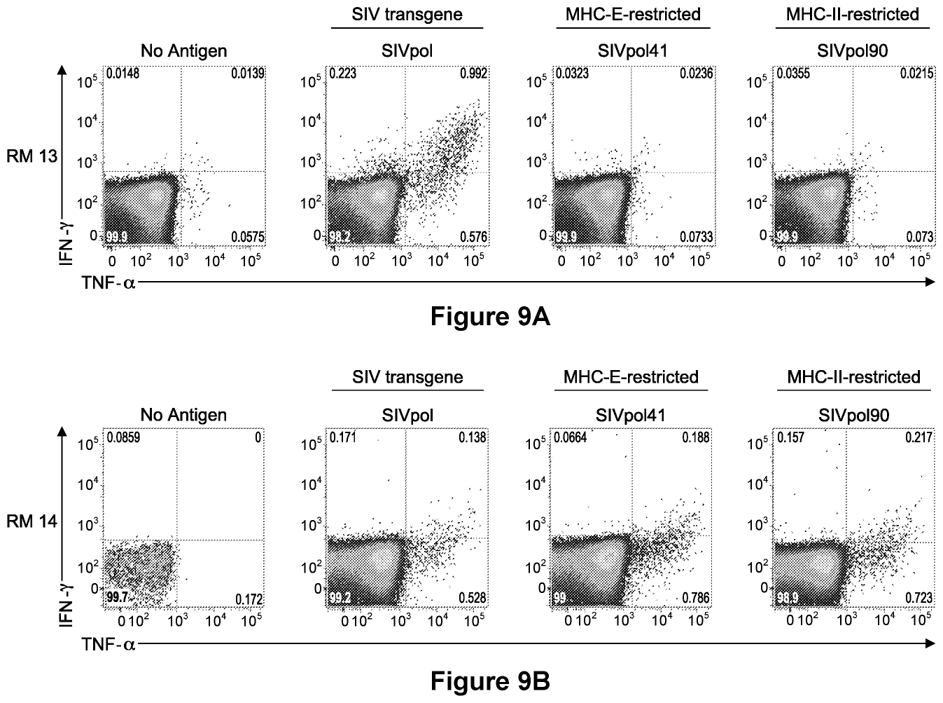

To determine whether the interaction with the inhibitory receptor LIR1 is responsible for the ability of UL18 to prevent the induction of MHC-II and MHC-E restricted CD8+ T cells the coding region of UL18 in construct 1 described above was mutated so that the amino acid aspartate at position 196 in the alpha-3 domain would be replaced with serine (D196S). Previous structural studies have shown that this aspartate is involved in binding of UL18 to LIR1 (Yang Z, Bjorkman P J. 2008. Structure of UL18, a peptide-binding viral MHC mimic, bound to a host inhibitory receptor. Proc Natl Acad Sci USA 105:10095-100). Moreover, this residue is conserved in all LIR1 binding HLA-molecules but absent in HLA-like molecules that do not bind LIR 1. The D196S mutant of UL18 was inserted into 68-1 RhCMV expressing SIVpol and the resulting construct was inoculated into two RM. On day 91, PBMC were isolated and the CD8+ T cell responses to the SIVpol was measured by ICS for IFNγ and TNFα using overlapping 15mer peptide pools that covered SIVpol or the SIVpol supertope peptide Pol41 (GFINTKEYKNVEIEV; SEQ ID NO: 33) or MHC-II supertope Pol90 (LPQGWKGSPAIFQYT; SEQ ID NO: 34). In contrast to animals inoculated with 68-1 RhCMV expressing intact UL18 (FIG. 9A), T cell responses to both SIVpol supertopes were observed in animals inoculated with 68-1 RhCMV expressing the D196S mutant of UL18 (FIG. 9B). These results thus indicated that UL18 needs to engage the LIR1 receptor to prevent induction of MHC-E and MHC-II restricted CD8+ T cells.

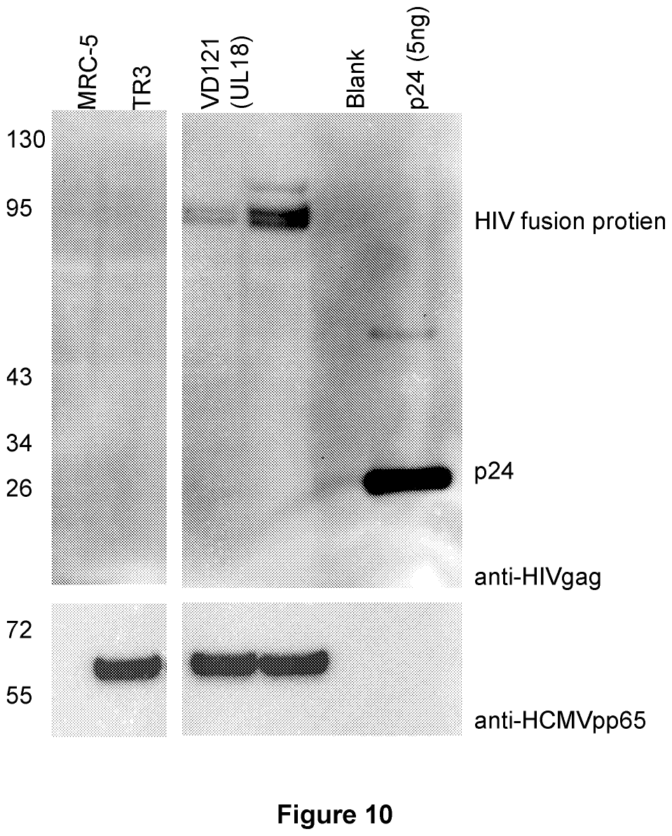

UL18 is considered to play a role in the evasion of NK cells (Prod'homme 2007. The human cytomegalovirus MEW class I homolog UL18 inhibits LIR-1+ but activates LIR-1− NK cells. J Immunol 178:4473-81). Since NK cell evasion can be crucial for vector function (Sturgill 2016. Natural Killer Cell Evasion Is Essential for Infection by Rhesus Cytomegalovirus. PLoS Pathog 12:e1005868) it was conceivable that deletion of UL18 from HCMV-based vectors would prevent their ability to elicit immune responses to heterologous antigens. To determine whether UL18-deleted HCMV is able to elicit T cell responses to an inserted antigen, UL18 was replaced with an HIV antigen, thereby deleting UL18 and using the endogenous UL18 promoter to drive expression of a HIVgag/nef/pol fusion protein. Additionally, the genes UL128, UL130, UL146, and UL147 were also deleted from the UL18-deleted vector, since the products of these genes were previously shown to inhibit MHC-E and MHC-II restricted CD8+ T cell responses (U.S. Pat. No. 10,532,099). As vector backbone we used HCMV TR3 (Caposio. 2019. Characterization of a live-attenuated HCMV-based vaccine platform. Scientific Reports 9: 19236). Expression of the HIV fusion protein in the resulting viral vector, (HCMV TR3 ΔUL18/HIVfusionΔUL128-130ΔUL146-147) was confirmed by immunoblot of human fibroblasts (FIG. 10).

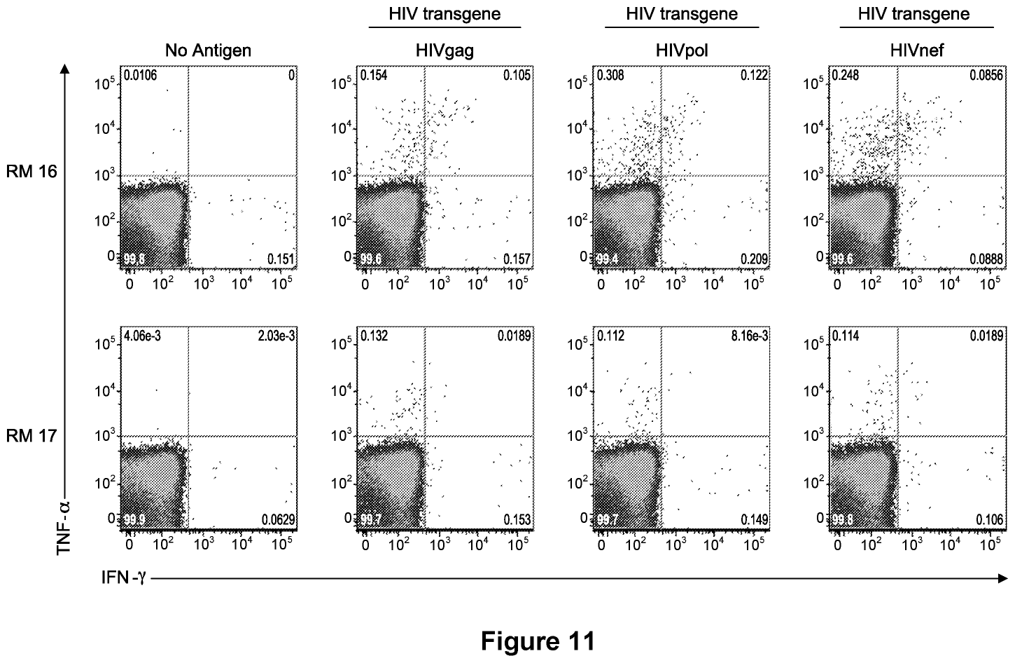

The UL18-deleted HCMV vector was also inoculated into a RM and the immune response to the HIV antigens was determined in PBMC by ICS on day 56 post-inoculation. As shown in FIG. 11, the vector elicited CD8+ T cell responses to HIVgag, HIVnef and HIVpol in RM as demonstrated by using overlapping peptide pools comprising each of these antigens. Therefore, we conclude that HCMV vectors lacking UL18 retain their ability to elicit T cell responses to heterologous antigens.

What is claimed is:

1. A recombinant HCMV vector comprising a nucleic acid sequence encoding heterologous antigen, wherein the recombinant HCMV vector does not express UL18.

2. The recombinant HCMV vector of claim 1, wherein the recombinant HCMV vector does not express UL128.

3. The recombinant HCMV vector of claim 1 or 2, wherein the recombinant HCMV vector does not express UL130.

4. The recombinant HCMV vector of any one of claims 1-3, wherein the recombinant HCMV vector does not express UL128 and UL130.

5. The recombinant HCMV vector of claim 4, wherein the recombinant HCMV vector does not express UL146 and UL147.

6. The recombinant HCMV vector of any one of claims 1-5, wherein the recombinant HCMV vector does not express a UL18 protein, UL128 protein, UL130 protein, UL146 protein, and UL147 protein, or orthologs thereof, due to the presence of one or more mutations in the nucleic acid sequence encoding UL18, UL128, UL130, UL146, or UL147.

7. The recombinant HCMV vector of claim 6, wherein the mutations in the nucleic acid sequence encoding UL18, UL128, UL130, UL146, or UL147 are selected from the group consisting of point mutations, frameshift mutations, truncation mutations, and deletion of all of the nucleic acid sequence encoding the viral protein.

8. The recombinant HCMV vector of any one of claims 1-7, wherein the recombinant HCMV vector further comprises a nucleic acid sequence encoding UL40, or an ortholog thereof.

9. The recombinant HCMV vector of any one of claims 1-8, wherein the recombinant HCMV vector further comprises a nucleic acid sequence encoding US28, or an ortholog thereof.

10. The recombinant HCMV vector of any one of claims 1-9, wherein the recombinant HCMV vector does not express UL82 (pp 71), or an ortholog thereof.

11. The recombinant HCMV vector of any one of claims 1-10, wherein the recombinant HCMV vector does not express US11, or an ortholog thereof.

12. The recombinant HCMV vector of any one of claims 1-10, wherein the recombinant HCMV vector further comprises a nucleic acid sequence encoding a microRNA (miRNA) recognition element (MRE), wherein the MRE contains a target site for a miRNA expressed in endothelial cells.

13. The recombinant HCMV vector of claim 12, wherein the miRNA expressed in endothelial cells is miR126, miR-126-3p, miR-130a, miR-210, miR-221/222, miR-378, miR-296, or miR-328.

14. The recombinant HCMV vector of any one of claims 1-11, wherein the recombinant HCMV vector further comprises a nucleic acid sequence encoding a MRE, wherein the MRE contains a target site for a miRNA expressed in myeloid cells.

15. The recombinant HCMV vector of claim 14, wherein the miRNA expressed in myeloid cells is miR-142-3p, miR-223, miR-27a, miR-652, miR-155, miR-146a, miR-132, miR-21, or miR-125.

16. The recombinant HCMV vector of any one of claims 1-15, wherein the heterologous antigen is a pathogen-specific antigen, a tumor antigen, a tissue-specific antigen, or a host self-antigen.

17. The recombinant HCMV vector of claim 16, wherein the pathogen-specific antigen is human immunodeficiency virus (HIV), herpes simplex virus type 1, herpes simplex virus type 2, hepatitis B virus, hepatitis C virus, papillomavirus, Plasmodium parasites, or Mycobacterium tuberculosis.

18. The recombinant HCMV vector of any one of claims 5-10 and 12-13, wherein the pathogen-specific antigen is an MHC-E supertope.

19. The recombinant HCMV vector of claim 18, wherein pathogen-specific antigen comprises a HIV epitope.

20. The recombinant HCMV vector of claim 19, wherein the HIV epitope is at least 80%, at least 85%, at least 90%, at least 95%, or 100% identical to LDAWEKIRLRPGGKK (SEQ ID NO: 13); DAWEKIRLR (SEQ ID NO: 14); KKAQQAAADTGNSSQ (SEQ ID NO: 15); KAQQAAADT (SEQ ID NO: 16); QMVHQAISPRTLNAW (SEQ ID NO: 17); HQAISPRTL (SEQ ID NO: 18); NTMLNTVGGHQAAMQ (SEQ ID NO: 19); VGGHQAAMQ (SEQ ID NO: 20); STLQEQIGWMTNNPP (SEQ ID NO: 21); STLQEQIGW (SEQ ID NO: 22); IVRMYSPVSILDIRQ (SEQ ID NO: 23); RMYSPVSIL (SEQ ID NO: 24); QKQEPIDKELYPLAS (SEQ ID NO: 25); KQEPIDKEL (SEQ ID NO: 26); SFSFPQITLWQRPLV (SEQ ID NO: 27); VRQYDQILIEICGKK (SEQ ID NO: 28); EPFRKQNPDIVIYQL (SEQ ID NO: 29); YVDGAANRETKLGKA (SEQ ID NO: 30); EEHEKYSNWRAMAS (SEQ ID NO: 31); or ILDLWVYHTQGYFPD (SEQ ID NO: 32).

21. The recombinant HCMV vector of claim 16, wherein the tumor antigen is related to acute myelogenous leukemia, chronic myelogenous leukemia, myelodysplastic syndrome, acute lymphoblastic leukemia, chronic lymphoblastic leukemia, non-Hodgkin's lymphoma, multiple myeloma, malignant melanoma, breast cancer, lung cancer, ovarian cancer, prostate cancer, pancreatic cancer, colon cancer, renal cell carcinoma (RCC), or germ cell tumors.

22. The recombinant HCMV vector of claim 16, wherein the host self-antigen is an antigen derived from the variable region of a T cell receptor (TCR) or an antigen derived from the variable region of a B cell receptor.

23. A pharmaceutical composition comprising the recombinant HCMV vector of any one of claims 1-22 and a pharmaceutically acceptable carrier.

24. An immunogenic composition comprising the recombinant HCMV vector of any one of claims 1-22 and a pharmaceutically acceptable carrier.

25. A method of generating an immune response in a subject to the at least one heterologous antigen, comprising administering to the subject the recombinant HCMV vector of any one of claims 1-22 in an amount effective to elicit a CD8+ T cell response to the at least one heterologous antigen.

26. Use of the recombinant HCMV vector of any one of claims 1-22 in the manufacture of a medicament for use in generating an immune response in a subject.

27. The recombinant HCMV vector of any of claims 1-22 for use in generating an immune response in a subject.

28. A method of treating or preventing cancer in a subject, comprising administering the recombinant HCMV vector of any one of claims 1-22 in an amount effective to elicit a CD8+ T cell response to the at least one heterologous antigen.

29. Use of the recombinant HCMV vector of any one of claims 1-22 in the manufacture of a medicament for use in treating or preventing cancer in a subject.

30. The recombinant HCMV vector of any one of claims 1-22 for use in treating or preventing cancer in a subject.

31. A method of treating or preventing a pathogenic infection in a subject, comprising administering to the subject the recombinant HCMV vector of any one of claims 1-22 in an amount effective to elicit a CD8+ T cell response to the at least one heterologous antigen.

32. Use of the recombinant HCMV vector of any one of claims 1-22 in the manufacture of a medicament for use in treating or preventing a pathogenic infection in a subject.

33. The recombinant HCMV vector of any one of claims 1-22 for use in treating or preventing a pathogenic infection in a subject.

34. A method of treating an autoimmune disease or disorder in a subject, comprising administering to the subject the recombinant HCMV vector of any one of claims 1-22 in an amount effective to elicit a CD8+ T cell response to the at least one heterologous antigen.

35. Use of the recombinant HCMV vector of claims 1-22 in the manufacture of a medicament for use in treating an autoimmune disease or disorder in a subject.

36. The recombinant HCMV vector of any one of claims 1-22 for use in treating an autoimmune disease or disorder in a subject.

37. The method, CMV vector for us, or use in manufacture of any one of claims 25-36, wherein at least 10% of the CD8+ T cells elicited by the recombinant HCMV vector are restricted by MHC-E or an ortholog thereof.

38. The method, CMV vector for us, or use in manufacture of claim 37, wherein at least 20%, at least 30%, at least 40%, at least 50%, at least 60%, at least 75%, at least 80%, at least 85%, at least 90%, or at least 95% of the CD8+ T cells elicited by the recombinant HCMV vector are restricted by MHC-E or an ortholog thereof.

39. The method, CMV vector for us, or use in manufacture of any one of claims 25-36, wherein at least 10% of the CD8+ T cells elicited by the recombinant HCMV vector are restricted by MHC-II or an ortholog thereof.

40. The method, CMV vector for us, or use in manufacture of claim 39, wherein at least 20%, at least 30%, at least 40%, at least 50%, at least 60% or at least 75% of the CD8+ T cells elicited by the recombinant HCMV vector are restricted by MHC-II or an ortholog thereof.

41. The method, CMV vector for us, or use in manufacture of any one of claims 25-36, wherein fewer than 10%, fewer than 20%, fewer than 30%, fewer than 40%, or fewer than 50% of the CD8+ T cells elicited by the recombinant HCMV vector are restricted by MHC-class Ia or an ortholog thereof.

42. The method, CMV vector for us, or use in manufacture of any one of claims 25-36, wherein at least 10% of the CD8+ T cells elicited by the recombinant HCMV vector are restricted by MHC-class Ia or an ortholog thereof.

43. The method, CMV vector for us, or use in manufacture of claim 42, wherein at least 20%, at least 30%, at least 40%, at least 50%, at least 60%, at least 75%, at least 80%, at least 85%, at least 90%, at least 95%, or the CD8+ T cells elicited by the recombinant HCMV vector are restricted by MHC-class Ia or an ortholog thereof.

44. The method, CMV vector for us, or use in manufacture of any one of claims 25-36, further comprising identifying a CD8+ TCR from the CD8+ T cells elicited by the recombinant HCMV vector, wherein the CD8+ TCR recognizes a MHC-II/heterologous antigen-derived peptide complex.

45. The method, CMV vector for us, or use in manufacture of any one of claims 25-36, further comprising identifying a CD8+ TCR from the CD8+ T cells elicited by the HCMV vector, wherein the CD8+ TCR recognizes a MHC-E/heterologous antigen-derived peptide complex.

46. The method, CMV vector for us, or use in manufacture of any one of claims 25-36, further comprising identifying a CD8+ TCR from the CD8+ T cells elicited by the HCMV vector, wherein the CD8+ TCR recognizes a MHC-class Ia/heterologous antigen-derived peptide complex.

47. The method, CMV vector for us, or use in manufacture of claim 44-46, wherein the CD8+ TCR is identified by DNA or RNA sequencing.

48. The method, CMV vector for us, or use in manufacture of claim 44, wherein the CD8+ TCR recognizes MHC-II supertopes.

49. The method, CMV vector for us, or use in manufacture of claim 45, wherein the CD8+ TCR recognizes MHC-E supertopes.

50. The method, CMV vector for us, or use in manufacture of claim 49, wherein the MHC-E supertope is an human immunodeficiency virus epitope.

51. The method, CMV vector for us, or use in manufacture of claim 50, wherein the MHC-E supertope is at least 80%, at least 85%, at least 90%, at least 95%, or is 100% identical to the amino acid sequence of LDAWEKIRLRPGGKK (SEQ ID NO: 13); DAWEKIRLR (SEQ ID NO: 14); KKAQQAAADTGNSSQ (SEQ ID NO: 15); KAQQAAADT (SEQ ID NO: 16); QMVHQAISPRTLNAW (SEQ ID NO: 17); HQAISPRTL (SEQ ID NO: 18); NTMLNTVGGHQAAMQ (SEQ ID NO: 19); VGGHQAAMQ (SEQ ID NO: 20); STLQEQIGWMTNNPP (SEQ ID NO: 21); STLQEQIGW (SEQ ID NO: 22); IVRMYSPVSILDIRQ (SEQ ID NO: 23); RMYSPVSIL (SEQ ID NO: 24); QKQEPIDKELYPLAS (SEQ ID NO: 25); KQEPIDKEL (SEQ ID NO: 26); SFSFPQITLWQRPLV (SEQ ID NO: 27); VRQYDQILIEICGKK (SEQ ID NO: 28); EPFRKQNPDIVIYQL (SEQ ID NO: 29); YVDGAANRETKLGKA (SEQ ID NO: 30); EEHEKYSNWRAMAS (SEQ ID NO: 31); or ILDLWVYHTQGYFPD (SEQ ID NO: 32).

52. A method of generating CD8+ T cells that recognize MHC-E-peptide complexes, the method comprising:
a. administering to a first subject the recombinant HCMV vector of any one of claim 5-10, 12-13, or 16-17 in an amount effective to generate a set of CD8+ T cells that recognize MHC-E/peptide complexes;
b. identifying a first CD8+ TCR from the set of CD8+ T cells, wherein the first CD8+ TCR recognizes a MHC-E/heterologous antigen-derived peptide complex;
c. isolating one or more CD8+ T cells from a second subject; and
d. transfecting the one or more CD8+ T cells with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3α and CDR3β of the first CD8+ TCR, thereby generating one or more CD8+ T cells that recognize MHC-E-peptide complexes.

53. A method of generating CD8+ T cells that recognize MHC-E-peptide complexes, the method comprising:
a. identifying a first CD8+ TCR from a set of CD8+ T cells, wherein the set of CD8+ T cells are generated from the recombinant HCMV vector of any one of claim 5-10, 12-13, or 16-17, wherein the first CD8+ TCR recognizes a MHC-E/heterologous antigen-derived peptide complex;
b. isolating one or more CD8+ T cells from a second subject; and
c. transfecting the one or more CD8+ T cells with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3α and CDR3β of the first CD8+ TCR, thereby generating one or more TCR-transgenic CD8+ T cells that recognize MHC-E-peptide complexes.

54. The method of claim 52 or 53, wherein the first CD8+ T cell recognizes MHC-E supertopes.

55. The method of claim 54, wherein the MHC-E supertopes comprise human immunodeficiency virus epitopes.

56. The method of any one of claims 54-55, wherein the MHC-E supertope is at least 80%, at least 85%, at least 90%, at least 95%, or is 100% identical to the amino acid sequence of
(SEQ ID NO: 13)
LDAWEKIRLRPGGKK;
(SEQ ID NO: 14)
DAWEKIRLR;
(SEQ ID NO: 15)
KKAQQAAADTGNSSQ;
(SEQ ID NO: 16)
KAQQAAADT;
(SEQ ID NO: 17)
QMVHQAISPRTLNAW;
(SEQ ID NO: 18)
HQAISPRTL;
(SEQ ID NO: 19)
NTMLNTVGGHQAAMQ;
(SEQ ID NO: 20)
VGGHQAAMQ;
(SEQ ID NO: 21)
STLQEQIGWMTNNPP;
(SEQ ID NO: 22)
STLQEQIGW;
(SEQ ID NO: 23)
IVRMYSPVSILDIRQ;
(SEQ ID NO: 24)
RMYSPVSIL;
(SEQ ID NO: 25)
QKQEPIDKELYPLAS;
(SEQ ID NO: 26)
KQEPIDKEL;
(SEQ ID NO: 27)
SFSFPQITLWQRPLV;
(SEQ ID NO: 28)
VRQYDQILIEICGKK;
(SEQ ID NO: 29)
EPFRKQNPDIVIYQL;
(SEQ ID NO: 30)
YVDGAANRETKLGKA;
(SEQ ID NO: 31)
EEHEKYSNWRAMAS;
or
(SEQ ID NO: 32)
ILDLWVYHTQGYFPD.

57. The method of any one of claims 52-56, wherein the second CD8+ T cell recognizes MHC-E supertopes.

58. The method of claim 57, wherein the MHC-E supertopes comprise human immunodeficiency virus epitopes.

59. The method of any one of claims 57-58, wherein the MHC-E supertope is at least 80%, at least 85%, at least 90%, at least 95%, or is 100% identical to the amino acid sequence of
(SEQ ID NO: 13)
LDAWEKIRLRPGGKK;
(SEQ ID NO: 14)
DAWEKIRLR;
(SEQ ID NO: 15)
KKAQQAAADTGNSSQ;
(SEQ ID NO: 16)
KAQQAAADT;
(SEQ ID NO: 17)
QMVHQAISPRTLNAW;
(SEQ ID NO: 18)
HQAISPRTL;
(SEQ ID NO: 19)
NTMLNTVGGHQAAMQ;
(SEQ ID NO: 20)
VGGHQAAMQ;
(SEQ ID NO: 21)
STLQEQIGWMTNNPP;
(SEQ ID NO: 22)
STLQEQIGW;
(SEQ ID NO: 23)
IVRMYSPVSILDIRQ;
(SEQ ID NO: 24)
RMYSPVSIL;
(SEQ ID NO: 25)
QKQEPIDKELYPLAS;
(SEQ ID NO: 26)
KQEPIDKEL;
(SEQ ID NO: 27)
SFSFPQITLWQRPLV;
(SEQ ID NO: 28)
VRQYDQILIEICGKK;
(SEQ ID NO: 29)
EPFRKQNPDIVIYQL;
(SEQ ID NO: 30)
YVDGAANRETKLGKA;
(SEQ ID NO: 31)
EEHEKYSNWRAMAS;
or
(SEQ ID NO: 32)
ILDLWVYHTQGYFPD.

60. The method of any one of claims 52-59, wherein the first CD8+ TCR is identified by DNA or RNA sequencing.

61. The method of any one of claims 52-60, wherein the nucleic acid sequence encoding the second CD8+ TCR is identical to the nucleic acid sequence encoding the first CD8+ TCR.

62. The method of any one of claims 52-61, wherein the first subject is a human.

63. The method of any one of claims 52-62, wherein the second subject is a human.

64. A method of generating CD8+ T cells that recognize MHC-E peptide complexes, the method comprising:
a. administering to a non-human primate a recombinant rhesus CMV (RhCMV) or cynomolgus CMV (CyCMV) vector deficient for orthologs of UL128, UL130, UL146, and UL147 and expressing HIV antigens in an amount effective to generate a set of CD8+ T cells that recognize MHC-E in complex with HIV supertope peptides of claims 18-20;
b. identifying a first CD8+ TCR from the set of CD8+ T cells, wherein the first recognizes a MHC-E/supertope peptide complex;
c. isolating one or more CD8+ T cells from a second subject; and
d. transfecting the one or more CD8+ T cells with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3α and CDR3β of the first CD8+ TCR, thereby generating one or more CD8+ T cells that recognize MHC-E peptide complexes.

65. A method of generating CD8+ T cells that recognize MHC-E peptide complexes, the method comprising:
a. identifying a first CD8+ TCR that recognizes a MHC-E/supertope peptide complex from a set of CD8+ T cells that recognize MHC-E in complex with the HIV supertope peptides of claims 18-20, wherein the set of CD8+ T cells are generated from a recombinant rhesus (RhCMV) or cynomolgus CMV (CyCCMV) vector deficient for orthologs of UL128, UL130, UL146, and UL147 and expressing HIV antigens in an amount effective to generate the set of CD8+ T cells;
b. isolating one or more CD8+ T cells from a second subject; and
c. transfecting the one or more CD8+ T cells with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3α and CDR3β of the first CD8+ TCR, thereby generating one or more CD8+ T cells that recognize MHC-E peptide complexes.

66. The method of claim 64 or 65, wherein the first subject is a nonhuman primate and the second subject is a human, and wherein the second CD8+ TCR is a chimeric nonhuman primate-human CD8+ TCR comprising the non-human primate CDR3α and CDR3β of the first CD8+ TCR.

67. The method of any one of claims 64-66, wherein the second CD8+ TCR comprises the non-human primate CDR1α, CDR2α, CDR3α, CDR1β, CDR2β, and CDR3β of the first CD8+ TCR.

68. The method of any one of claims 64-67, wherein the second CD8+ TCR comprises CDR1α, CDR2α, CDR3α, CDR1β, CDR2β, and CDR3β of the first CD8+ TCR.

69. The method of any one of claims 64-68, wherein the second CD8+ TCR is a chimeric CD8+ TCR.

70. The method of any one of claims 64-69, wherein administering the recombinant HCMV vector to the first subject comprises intravenous, intramuscular, intraperitoneal, or oral administration of the recombinant HCMV vector to the first subject.

71. The method of any one of claims 52-70, further comprising administering the transfected CD8+ T cells to the second subject to treat or prevent cancer.

72. The method of claim 71, wherein the cancer is acute myelogenous leukemia, chronic myelogenous leukemia, myelodysplastic syndrome, acute lymphoblastic leukemia, chronic lymphoblastic leukemia, non-Hodgkin's lymphoma, multiple myeloma, malignant melanoma, breast cancer, lung cancer, ovarian cancer, prostate cancer, pancreatic cancer, colon cancer, renal cell carcinoma (RCC), or germ cell tumors.

73. The method of any one of claims 52-70, further comprising administering the transfected CD8+ T cells to the second subject to treat or prevent a pathogenic infection.

74. The method of claim 73, wherein the pathogenic infection is caused by human immunodeficiency virus, herpes simplex virus type 1, herpes simplex virus type 2, hepatitis B virus, hepatitis C virus, papillomavirus, Plasmodium parasites, or Mycobacterium tuberculosis.

75. The method of any one of claims 52-70, further comprising administering the transfected CD8+ T cells to the subject to induce an autoimmune response to the host self-antigen.

76. A method of generating CD8+ T cells that recognize MHC-II-peptide complexes, the method comprising:
a. administering to a first subject the recombinant HCMV vector of any one of claim 1-11, 14, or 15 in an amount effective to generate a set of CD8+ T cells that recognize MHC-II/peptide complexes;
b. identifying a first CD8+ TCR from the set of CD8+ T cells, wherein the first CD8+ TCR recognizes a MHC-II/heterologous antigen-derived peptide complex;
c. isolating one or more CD8+ T cells from a second subject; and
d. transfecting the one or more CD8+ T cells with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3α and CDR3β of the first CD8+ TCR, thereby generating one or more CD8+ T cells that recognize a MHC-II peptide complexes.

77. A method of generating CD8+ T cells that recognize MHC-II-peptide complexes, the method comprising:
a. identifying a first CD8+ TCR that recognizes a MHC-II/heterologous antigen-derived peptide complex from a set of CD8+ T cells that recognize MHC-II/peptide complexes, wherein the set of CD8+ T cells are generated from the recombinant HCMV vector of any one of claim 1-11, 14, or 15;
b. isolating one or more CD8+ T cells from a second subject; and
c. transfecting the one or more CD8+ T cells with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3α and CDR3β of the first CD8+ TCR, thereby generating one or more CD8+ T cells that recognize a MHC-II peptide complexes.

78. The method of any one of claims 76-77, wherein the first CD8+ T cell recognizes MHC-II supertopes.

79. The method of any one of claims 76-78, wherein the second CD8+ T cell recognizes MHC-II supertopes.

80. The method of any one of claims 76-79, wherein the first CD8+ TCR is identified by DNA or RNA sequencing.

81. The method of any one of claims 76-80, wherein the nucleic acid sequence encoding the second CD8+ TCR is identical to the nucleic acid sequence encoding the first CD8+ TCR.

82. The method of any one of claims 76-81, wherein the first subject is a human.

83. The method of any one of claims 76-82, wherein the second subject is a human.

84. The method of claim 76-83, wherein administering the HCMV vector to the first subject comprises intravenous, intramuscular, intraperitoneal, or oral administration of the HCMV vector to the first subject.

85. The method of any one of claims 76-84, further comprising administering the transfected CD8+ T cells to the second subject to treat or prevent cancer.

86. The method of claim 85, wherein the cancer is selected from the group consisting of acute myelogenous leukemia, chronic myelogenous leukemia, myelodysplastic syndrome, acute lymphoblastic leukemia, chronic lymphoblastic leukemia, non-Hodgkin's lymphoma, multiple myeloma, malignant melanoma, breast cancer, lung cancer, ovarian cancer, prostate cancer, pancreatic cancer, colon cancer, renal cell carcinoma (RCC), and germ cell tumors.

87. The method of any one of claims 76-84, further comprising administering the transfected CD8+ T cells to the second subject to treat or prevent a pathogenic infection.

88. The method of claim 87 wherein the pathogenic infection is caused by a pathogen selected from the group consisting of human immunodeficiency virus, herpes simplex virus type 1, herpes simplex virus type 2, hepatitis B virus, hepatitis C virus, papillomavirus, Plasmodium parasites, and Mycobacterium tuberculosis.

89. The method of any one of claims 76-84, further comprising administering the transfected CD8+ T cells to the subject to induce an autoimmune response to the host self-antigen.

90. A method of generating CD8+ T cells that recognize MHC-I-peptide complexes, the method comprising:
a. administering to a first subject the recombinant HCMV vector of any one of claims 1-11 in an amount effective to generate a set of CD8+ T cells that recognize MHC-I/peptide complexes;
b. identifying a first CD8+ TCR from the set of CD8+ T cells, wherein the first CD8+ TCR recognizes a MHC-I/heterologous antigen-derived peptide complex;
c. isolating one or more CD8+ T cells from a second subject; and
d. transfecting the one or more CD8+ T cells with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3α and CDR3β of the first CD8+ TCR, thereby generating one or more CD8+ T cells that recognize MHC-I peptide complexes.

91. A method of generating CD8+ T cells that recognize MHC-I-peptide complexes, the method comprising:
a. identifying a first CD8+ TCR that recognizes a MHC-I/heterologous antigen-derived peptide complex from a set of CD8+ T cells that recognize a MHC-I/heterologous antigen-derived peptide complex, wherein the set of CD8+ T cells are generated from the recombinant HCMV vector of any one of claims 1-11;
b. isolating one or more CD8+ T cells from a second subject; and
c. transfecting the one or more CD8+ T cells with an expression vector, wherein the expression vector comprises a nucleic acid sequence encoding a second CD8+ TCR and a promoter operably linked to the nucleic acid sequence encoding the second CD8+ TCR, wherein the second CD8+ TCR comprises CDR3α and CDR3β of the first CD8+ TCR, thereby generating one or more CD8+ T cells that recognize MHC-I peptide complexes.

92. The method of any one of claims 90-91, wherein the first CD8+ TCR is identified by DNA or RNA sequencing.

93. The method of any one of claims 90-92, wherein the nucleic acid sequence encoding the second CD8+ TCR is identical to the nucleic acid sequence encoding the first CD8+ TCR.

94. The method of any one of claims 90-93, wherein the first subject is a human.

95. The method of any one of claims 90-94, wherein the second subject is a human.

96. The method of any one of claims 90-95, wherein administering the HCMV vector to the first subject comprises intravenous, intramuscular, intraperitoneal, or oral administration of the HCMV vector to the first subject.

97. The method of any one of claims 90-96, further comprising administering the transfected CD8+ T cells to the second subject to treat or prevent cancer.

98. The method of claim 97, wherein the cancer is selected from the group consisting of acute myelogenous leukemia, chronic myelogenous leukemia, myelodysplastic syndrome, acute lymphoblastic leukemia, chronic lymphoblastic leukemia, non-Hodgkin's lymphoma, multiple myeloma, malignant melanoma, breast cancer, lung cancer, ovarian cancer, prostate cancer, pancreatic cancer, colon cancer, renal cell carcinoma (RCC), and germ cell tumors.

99. The method of any one of claims 90-96, further comprising administering the transfected CD8+ T cells to the second subject to treat or prevent a pathogenic infection.

100. The method of claim 99, wherein the pathogenic infection is caused by a pathogen selected from the group consisting of human immunodeficiency virus, herpes simplex virus type 1, herpes simplex virus type 2, hepatitis B virus, hepatitis C virus, papillomavirus, Plasmodium parasites, and Mycobacterium tuberculosis.

101. The method of any one of claims 90-96, further comprising administering the transfected CD8+ T cells to the subject to induce an autoimmune response to the host self-antigen.

102. A CD8+ T cell generated by the method of claims 25-101.

103. The CD8+ T cell of claim 102, wherein the pathogen-specific antigen is human immunodeficiency virus, simian immunodeficiency virus, herpes simplex virus type 1, herpes simplex virus type 2, hepatitis B virus, hepatitis C virus, papillomavirus, Plasmodium parasites, or Mycobacterium tuberculosis.

104. The CD8+ T cell of claim 102, wherein the tumor antigen is related acute myelogenous leukemia, chronic myelogenous leukemia, myelodysplastic syndrome, acute lymphoblastic leukemia, chronic lymphoblastic leukemia, non-Hodgkin's lymphoma, multiple myeloma, malignant melanoma, breast cancer, lung cancer, ovarian cancer, prostate cancer, pancreatic cancer, colon cancer, renal cell carcinoma (RCC), or germ cell tumors.

105. The CD8+ T cell of claim 102, wherein the host self-antigen is an antigen derived from the variable region of a T cell receptor (TCR) or an antigen derived from the variable region of a B cell receptor.

106. A method of treating or preventing a pathogenic infection in a subject, the method comprising administering the CD8+ T cell of claim 102 or 103 to the subject.

107. Use of the CD8+ T cell of claim 102 or 103 in the manufacture of a medicament for use in treating or preventing a pathogenic infection in a subject.

108. The CD8+ T cell of claim 102 or 103 for use in treating or preventing a pathogenic infection in a subject.

109. A method of treating or preventing cancer in a subject, the method comprising administering the CD8+ T cell of claim 102 or 104 to the subject.

110. Use of the CD8+ T cell of claim 102 or 104 in the manufacture of a medicament for use in treating or preventing cancer in a subject.

111. The CD8+ T cell of claim 102 or 104 for use in treating or preventing cancer in a subject.

112. A method of treating an autoimmune disease or disorder, the method comprising administering the CD8+ T cell of claim 102 or 105 to the subject.

113. Use of the CD8+ T cell of claim 102 or 105 in the manufacture of a medicament for use in treating an autoimmune disease or disorder.

114. The CD8+ T cell of claim 102 or 105 for use in treating an autoimmune disease or disorder.

115. A method of inducing an autoimmune response to a host self-antigen, the method comprising administering the CD8+ T cell of claim 102 or 105 to the subject.

116. A human immunodeficiency virus antigen between 9 and 15 amino acids in length and that is at least 90%, at least 95%, or 100% identical to the amino acid sequence of
(SEQ ID NO: 13)
LDAWEKIRLRPGGKK;
(SEQ ID NO: 14)
DAWEKIRLR;
(SEQ ID NO: 15)
KKAQQAAADTGNSSQ;
(SEQ ID NO: 16)
KAQQAAADT;
(SEQ ID NO: 17)
QMVHQAISPRTLNAW;
(SEQ ID NO: 18)
HQAISPRTL;
(SEQ ID NO: 19)
NTMLNTVGGHQAAMQ;
(SEQ ID NO: 20)
VGGHQAAMQ;
(SEQ ID NO: 21)
STLQEQIGWMTNNPP;
(SEQ ID NO: 22)
STLQEQIGW;
(SEQ ID NO: 23)
IVRMYSPVSILDIRQ;
(SEQ ID NO: 24)
RMYSPVSIL;
(SEQ ID NO: 25)
QKQEPIDKELYPLAS;
(SEQ ID NO: 26)
KQEPIDKEL;
(SEQ ID NO: 27)
SFSFPQITLWQRPLV;
(SEQ ID NO: 28)
VRQYDQILIEICGKK;
(SEQ ID NO: 29)
EPFRKQNPDIVIYQL;
(SEQ ID NO: 30)
YVDGAANRETKLGKA;
(SEQ ID NO: 31)
EEHEKYSNWRAMAS;
or
(SEQ ID NO: 32)
ILDLWVYHTQGYFPD.

117. A recombinant HCMV vector comprising a nucleic acid encoding one or more of the human immunodeficiency virus antigens of claim 116.

118. The recombinant HCMV vector of claim 117, wherein the recombinant HCMV vector does not express UL18.

119. The recombinant HCMV vector of claim 117 or 118, wherein the recombinant HCMV vector does not express UL128.

120. The recombinant HCMV vector of any one of claims 117-119, wherein the recombinant HCMV vector does not express UL130.

121. The recombinant HCMV vector of any one of claims 117-120, wherein the recombinant HCMV vector does not express UL128 and UL130.

122. The recombinant HCMV vector of claim 121, wherein the recombinant HCMV vector does not express UL146 and UL147.

123. The recombinant HCMV vector of any one of claims 117-122, wherein the recombinant HCMV vector does not express UL18 protein, UL128 protein, UL130 protein, UL146 protein, and UL147 protein, or orthologs thereof, due to the presence of one or more mutations in the nucleic acid sequence encoding UL18, UL128, UL130, UL146, or UL147.

124. The recombinant HCMV vector of claim 123, wherein the mutations in the nucleic acid sequence encoding UL18, UL128, UL130, UL146, or UL147 are selected from the group consisting of point mutations, frameshift mutations, truncation mutations, and deletion of all of the nucleic acid sequence encoding the viral protein.

125. The recombinant HCMV vector of any one of claims 117-124, wherein the recombinant HCMV vector further comprises a nucleic acid sequence encoding UL40, or an ortholog thereof.

126. The recombinant HCMV vector of any one of claims 117-125, wherein the recombinant HCMV vector further comprises a nucleic acid sequence encoding US28, or an ortholog thereof.

127. The recombinant HCMV vector of any one of claims 117-126, wherein the recombinant HCMV vector does not express UL82 (pp 71), or an ortholog thereof.

128. The recombinant HCMV vector of any one of claims 117-127, wherein the recombinant HCMV vector does not express US11, or an ortholog thereof.

129. The recombinant HCMV vector of any one of claims 117-128, wherein the recombinant HCMV vector further comprises a nucleic acid sequence encoding a microRNA (miRNA) recognition element (MRE), wherein the MRE contains a target site for a miRNA expressed in endothelial cells.

130. The recombinant HCMV vector of claim 129, wherein the miRNA expressed in endothelial cells is miR126, miR-126-3p, miR-130a, miR-210, miR-221/222, miR-378, miR-296, or miR-328.

131. The recombinant HCMV vector of any one of claims 117-130, wherein the recombinant HCMV vector further comprises a nucleic acid sequence encoding a MRE, wherein the MRE contains a target site for a miRNA expressed in myeloid cells.

132. The recombinant HCMV vector of claim 131, wherein the miRNA expressed in myeloid cells is miR-142-3p, miR-223, miR-27a, miR-652, miR-155, miR-146a, miR-132, miR-21, or miR-125.


SEQUENCE LISTING (34 sequences)

SEQ ID NO: 1 - protein, 23 aa, Artificial Sequence
  SIVrev MHC-E supertrope
RRWRRRWQQLLALADRIYSFPDP

SEQ ID NO: 2 - protein, 19 aa, Artificial Sequence
  SIVtat MHC-E supertrope
TSSASNKPISNRTRHCQPE

SEQ ID NO: 3 - protein, 19 aa, Artificial Sequence
  SIVnef MHC-E supertrope
ISMRRSRPSGDLRQRLLRA

SEQ ID NO: 4 - protein, 19 aa, Artificial Sequence
  SIVnef MHC-E supertrope
EKLAYRKQNMDDIDEEDDD

SEQ ID NO: 5 - protein, 19 aa, Artificial Sequence
  SIVnef MHC-E supertrope
AQTSQWDDPWGEVLAWKFD

SEQ ID NO: 6 - protein, 19 aa, Artificial Sequence
  SIVnef MHC-E supertrope
YVRYPEEFGSKSGLSEEEV

SEQ ID NO: 7 - protein, 23 aa, Artificial Sequence
  SIVpol MHC-Ia supertrope
GGIGGFINTKEYKNVEIEVLGKR

SEQ ID NO: 8 - protein, 23 aa, Artificial Sequence
  SIVpol MHC-Ia supertrope
NTPTFAIKKKDKNKWRMLIDFRE

SEQ ID NO: 9 - protein, 19 aa, Artificial Sequence
  SIVpol MHC-Ia supertrope
WMGYELWPTKWKLQKIELP

SEQ ID NO: 10 - protein, 19 aa, Artificial Sequence
  SIVgag MHC-II supertrope
LGLQKCVRMYNPTNILDVK

SEQ ID NO: 11 - protein, 23 aa, Artificial Sequence
  SIVgag MHC-II supertrope
YMQLGKQQREKQRESREKPYKEV

SEQ ID NO: 12 - protein, 235 aa, Artificial Sequence
  Supertrope fusion protein
MRRWRRRWQQLLALADRIYSFPDPTSSASNKPISNRTRHCQPEISMRRSRPSGDLRQRLLRAEKLAYRKQNMDDIDEEDDDAQTSQWDDPWGEVLAWKFDYVRYPEEFGSKSGLSEEEVGGIGGFINTKEYKNVEIVLGKRNTPTFAIKKKDKNKWRMLIDFREWMGYELWPTKWKLQKIELPLGLQKCVRMYNPTNILDVKYMQLGKQQREKQRESREKPYKEVYPYDVPDYAD

SEQ ID NO: 13 - protein, 15 aa, Artificial Sequence
  HIVgag peptide 4 full peptide sequence
LDAWEKIRLRPGGKK

SEQ ID NO: 14 - protein, 9 aa, Artificial Sequence
  HIVgag peptide 4 optimal minimal sequence
DAWEKIRLR

SEQ ID NO: 15 - protein, 15 aa, Artificial Sequence
  HIVgag peptide 29 full peptide sequence
KKAQQAAADTGNSSQ

SEQ ID NO: 16 - protein, 9 aa, Artificial Sequence
  HIVgag peptide 29 optimal minimal sequence
KAQQAAADT

SEQ ID NO: 17 - protein, 15 aa, Artificial Sequence
  HIVgag peptide 36 full peptide sequence
QMVHQAISPRTLNAW

SEQ ID NO: 18 - protein, 9 aa, Artificial Sequence
  HIVgag peptide 36 optimal minimal sequence
HQAISPRTL

SEQ ID NO: 19 - protein, 15 aa, Artificial Sequence
  HIVgag peptide 47 full peptide sequence
NTMLNTVGGHQAAMQ

SEQ ID NO: 20 - protein, 9 aa, Artificial Sequence
  HIVgag peptide 37 optimal minimal sequence
VGGHQAAMQ

SEQ ID NO: 21 - protein, 15 aa, Artificial Sequence
  HIVgag peptide 61 full peptide sequence
STLQEQIGWMTNNPP

SEQ ID NO: 22 - protein, 9 aa, Artificial Sequence
  HIVgag peptide 61 optimal minimal sequence
STLQEQIGW

SEQ ID NO: 23 - protein, 15 aa, Artificial Sequence
  HIVgag peptide 69 full peptide sequence
IVRMYSPVSILDIRQ

SEQ ID NO: 24 - protein, 9 aa, Artificial Sequence
  HIVgag peptide 69 optimal minimal sequence
RMYSPVSIL

SEQ ID NO: 25 - protein, 15 aa, Artificial Sequence
  HIVgag peptide 119 full peptide sequence
QKQEPIDKELYPLAS

SEQ ID NO: 26 - protein, 9 aa, Artificial Sequence
  HIVgag peptide optimal minimal sequence
KQEPIDKEL

SEQ ID NO: 27 - protein, 15 aa, Artificial Sequence
  HIVpol peptide 14 full peptide sequence
SFSFPQITLWQRPLV

SEQ ID NO: 28 - protein, 15 aa, Artificial Sequence
  HIVpol peptide 28 full peptide sequence
VRQYDQILIEICGKK

SEQ ID NO: 29 - protein, 15 aa, Artificial Sequence
  HIVpol peptide 81 full peptide sequence
EPFRKQNPDIVIYQL

SEQ ID NO: 30 - protein, 15 aa, Artificial Sequence
  HIVpol peptide 148 full peptide sequence
YVDGAANRETKLGKA

SEQ ID NO: 31 - protein, 14 aa, Artificial Sequence
  HIVpol peptide 180 full peptide sequence
EEHEKYSNWRAMAS

SEQ ID NO: 32 - protein, 15 aa, Artificial Sequence
  HIVnef peptide 28 full peptide sequence
ILDLWVYHTQGYFPD

SEQ ID NO: 33 - protein, 15 aa, Artificial Sequence
  POL #41
GFINTKEYKNVEIEV

SEQ ID NO: 34 - protein, 15 aa, Artificial Sequence
  pol #90
LPQGWKGSPAIFQYT